(19) United States
(12) Patent Application Publication
(10) Pub. No.: US 2021/0301328 A1
(43) Pub. Date: Sep. 30, 2021

(54) COMPOSITIONS AND METHODS FOR DIGITAL POLYMERASE CHAIN REACTION

(71) Applicant: ACCURAGEN HOLDINGS LIMITED, Grand Cayman (KY)

(72) Inventors: Li WENG, Fremont, CA (US); Malek FAHAM, Burlingame, CA (US); Paul Ling-Fung TANG, San Francisco, CA (US); Shengrong LIN, Fremont, CA (US)

(21) Appl. No.: 17/127,550

(22) Filed: Dec. 18, 2020

Related U.S. Application Data
(63) Continuation of application No. PCT/US2019/040610, filed on Jul. 3, 2019.
(60) Provisional application No. 62/694,324, filed on Jul. 5, 2018.

(51) Int. Cl. C12Q 1/6827; C12Q 1/686

(52) U.S. Cl. CPC C12Q 1/6827; C12Q 1/686

(57) ABSTRACT

In some aspects, the present disclosure provides methods for identifying sequence variants in a nucleic acid sample. In some embodiments, a method comprises distinguishing between a true mutation in a polynucleotide and a random error introduced during an amplification step. In some embodiments, the methods reduce the number of false positives reported by a digital PCR assay. In some embodiments, the methods improve the accuracy of a digital PCR assay.

### CROSS-REFERENCE

This application is a continuation of PCT International Application No. PCT/US19/40610 filed on Jul. 3, 2019, which claims the benefit of U.S. Provisional Application No. 62/694,324 filed on Jul. 5, 2018, each of which are incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 18, 2020, is named 47608710301SL.txt and is 10 kilobytes in size.

### BACKGROUND OF THE INVENTION

Digital polymerase chain reaction (PCR) is a modification of traditional polymerase chain reaction methods that allows a user to directly quantify nucleic acids in a sample. Digital PCR methods generally involve partitioning a sample into a plurality of discrete partitions, such that each partition can be interrogated individually. Digital PCR is very sensitive but may be hard to scale due to the limited plex one can assay in one reaction. This issue may be more problematic with liquid biopsy using cell-free DNA (cfDNA) as input, as the starting material usually is low. One approach to solve this problem may be to amplify the cfDNA before performing digital PCR in order to provide enough starting material to split into different assays. However, errors introduced during the amplification step may create false positive calls by digital PCR. This may be challenging for low allele frequency variant detection. Accordingly, provided herein are compositions and methods for performing digital polymerase chain reaction on a sample with small amounts of nucleic acids. The compositions and methods may provide improvement to digital PCR techniques by reducing the number of false positive calls.

### SUMMARY OF THE INVENTION

In one aspect, a method is provided for identifying a sequence variant in a nucleic acid sample comprising a plurality of polynucleotides, the method comprising: (a) circularizing the plurality of polynucleotides to form a plurality of circularized polynucleotides; (b) amplifying the plurality of circularized polynucleotides to generate a plurality of concatemers, each comprising a plurality of sequence repeats; (c) partitioning the plurality of concatemers into a plurality of partitions, such that, on average, no more than one concatemer comprising a target sequence is present in an individual partition, wherein an individual partition of the plurality of partitions contains at least one of a first probe and a second probe, wherein the first probe binds to the target sequence that lacks the sequence variant and produces a first signal, and the second probe binds to the target sequence that contains the sequence variant and produces a second signal; (d) detecting the first signal and the second signal from the individual partition; and (e) identifying the sequence variant as present in the target sequence only when a level of the second signal exceeds that of a threshold level indicative of one copy of a target sequence, and a level of the first signal is below that of a threshold level indicative of one copy of a target sequence. In some cases, the method further comprises identifying the sequence variant as absent when a level of the first signal exceeds that of a threshold level indicative of one copy of a target sequence and a level of the second signal is below that of a threshold level indicative of one copy of a target sequence. In some cases, the method further comprises identifying a false positive when a level of the first signal exceeds that of a threshold level indicative of one copy of a target sequence, and a level of the second signal exceeds that of a threshold level indicative of one copy of a target sequence. In some cases, the method further comprises outputting a result based on the identifying. In some cases, the false positives are omitted from the result. In some cases, the plurality of polynucleotides comprise single-stranded polynucleotides. In some cases, the plurality of polynucleotides comprise cell-free DNA. In some cases, the circularizing comprises ligating a 5′ end and a 3′ end of at least one of the plurality of polynucleotides. In some cases, the circularizing comprises ligating an adapter to the 5′ end, the 3′ end, or both the 5′ end and the 3′ end of at least one of the plurality of polynucleotides. In some cases, the amplifying comprises amplifying using a polymerase having strand-displacement activity. In some cases, the amplifying comprises amplifying the plurality of circularized polynucleotides using rolling circle amplification. In some cases, the amplifying comprises subjecting the plurality of circular polynucleotides to an amplification reaction mixture comprising random primers. In some cases, the amplifying comprises subjecting the plurality of circular polynucleotides to an amplification reaction mixture comprising one or more primers, each of which specifically hybridizes to a different target sequence via sequence complementarity. In some cases, the plurality of concatemers are not enriched prior to the partitioning. In some cases, the method further comprises prior to the partitioning, fragmenting the plurality of concatemers to generate a plurality of fragmented concatemers. In some cases, the method further comprises after the fragmenting and prior to the partitioning, selecting a plurality of the fragmented concatemers based on size. In some cases, the plurality of partitions comprise emulsion-based droplets. In some cases, the emulsion-based droplets comprise picoliter- or nanoliter-sized droplets. In some cases, the plurality of partitions comprise a well or a tube. In some cases, the first probe comprises a first detectable label and the second probe comprises a second detectable label. In some cases, the first detectable label comprises a first fluorescent label and the second detectable label comprises a second fluorescent label. In some cases, an emission spectrum of the first fluorescent label and the second fluorescent label are different. In some cases, the detecting further comprises measuring an intensity of the first signal and the second signal. In some cases, the sequence variant is a single nucleotide polymorphism. In some cases, the first probe and the second probe are Taqman assay-based probes. In some cases, the method further comprises after the partitioning and before the detecting, performing a polymerase chain reaction on the concatemers to amplify a region of the plurality of sequence repeats.

In another aspect, a method is provided for reducing error in a digital polymerase chain reaction on a nucleic acid sample comprising less than 50 ng of polynucleotides, the method comprising: (a) circularizing individual polynucleotides in the nucleic acid sample to generate a plurality of circularized polynucleotides; (b) amplifying the plurality of circularized polynucleotides to form a plurality of concatemers, each comprising a plurality of sequence repeats; (c) partitioning the plurality of concatemers into a plurality of partitions, such that, on average, no more than one concatemer comprising a target sequence is present in an individual partition, wherein an individual partition of the plurality of partitions contains at least one of a first probe and a second probe, wherein the first probe binds to the plurality of sequence repeats that lack the sequence variant and produces a first signal, and the second probe binds to the plurality of sequence repeats that contain the sequence variant and produces a second signal; (d) detecting the first signal and the second signal from the individual partition; and (e) identifying a false positive when a level of the first signal exceeds that of a threshold level indicative of one copy of a target sequence, and a level of the second signal exceeds that of a threshold level indicative of one copy of a target sequence. In some cases, the method further comprises outputting a result. In some cases, the result excludes the false positive. In some cases, the method reduces false positives by at least 20%. In some cases, the nucleic acid sample comprises cell-free polynucleotides. In some cases, the cell-free polynucleotides comprise circulating tumor DNA. In some cases, the nucleic acid sample is from a subject. In some cases, the nucleic acid sample is urine, blood, stool, saliva, tissue, or bodily fluid.

In another aspect, a system is provided for detecting a sequence variant, the system comprising: (a) a computer configured to receive a user request to perform a detection reaction on a sample; (b) an amplification system that performs a nucleic acid amplification reaction on the sample or a portion thereof in response to the user request, wherein the amplification reaction comprises: (i) circularizing individual polynucleotides of the sample to form a plurality of circularized polynucleotides; and (ii) amplifying the plurality of circularized polynucleotides to form a plurality of concatemers, each comprising a plurality of sequence repeats; (c) a partitioning system that partitions the plurality of concatemers into a plurality of partitions, such that, on average, no more than one concatemer comprising a target sequence is present in an individual partition; and (d) a detection system that detects a level of a first signal and a level of a second signal from an individual partition, wherein the first signal is generated when a first probe binds to the plurality of sequence repeats that lack the sequence variant, and the second signal is generated when a second probe binds to the plurality of sequence repeats that contain the sequence variant; and (e) a report generator that sends a report to a recipient, wherein the report contains results for detection of the sequence variant. In some cases, the report identifies a presence of the sequence variant when a level of the second signal exceeds that of a threshold level indicative of one copy of a target sequence, and a level of the first signal is below that of a threshold level indicative of one copy of a target sequence. In some cases, the report identifies an absence of the sequence variant when a level of the first signal exceeds that of a threshold level indicative of one copy of a target sequence, and a level of the second signal is below that of a threshold level inactive of one copy of a target sequence. In some cases, the report identifies a false positive when a level of the first signal exceeds that of a threshold level indicative of one copy of a target sequence, and a level of the second signal exceeds that of a threshold level indicative of one copy of a target sequence

In another aspect, a computer-readable medium is provided comprising codes that, upon execution by one or more processors, implement a method of detecting a sequence variant, the method comprising: (a) receiving a user request to perform a detection reaction on a sample; (b) performing a nucleic acid amplification reaction on the sample or a portion thereof in response to the user request, wherein the amplification reaction comprises: (i) circularizing individual polynucleotides of the sample to form a plurality of circularized polynucleotides; and (ii) amplifying the plurality of circularized polynucleotides to form a plurality of concatemers, each comprising a plurality of sequence repeats; (c) partitioning the plurality of concatemers into a plurality of partitions, such that, on average, no more than one concatemer comprising a target sequence is present in an individual partition, wherein an individual partition of the plurality of partitions contains at least one of a first probe and a second probe, wherein the first probe binds to the plurality of sequence repeats that lack the sequence variant and produces a first signal, and the second probe binds to the plurality of sequence repeats that contain the sequence variant and produces a second signal; (d) detecting the first signal and the second signal from the individual partition; and (e) identifying the sequence variant as present only when a level of the second signal exceeds that of threshold level indicative of one copy of a target sequence, and a level of the first signal is below that of a threshold level indicative of one copy of a target sequence; and (f) generating a report that contains results for detection of the sequence variant. In some cases, the method further comprises identifying the sequence variant as absent when a level of the first signal exceeds that of a threshold level indicative of one copy of a sequence variant, and a level of the second signal is below that of a threshold level indicative of one copy of a sequence variant. In some cases, the method further comprises identifying a false positive when a level of the first signal exceeds that of a threshold level indicative of one copy of a target sequence, and a level of the second signal exceeds that of a threshold level indicative of one copy of a target sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an example methodology for performing digital PCR in accordance with embodiments of the disclosure.

FIG. 2 depicts three embodiments associated with the formation of circularized cDNA. At the top, single-stranded DNA (ssDNA) is circularized in the absence of adapters, while the middle scheme depicts the use of adapters, and the bottom scheme utilizes two adapter oligos (yielding different sequences on each end) and may further include a splint oligo that hybridizes to both adapters to bring the two ends in proximity.

FIG. 3A and FIG. 3B depict two schemes for the addition of adapters using blocked ends of the nucleic acids.

FIG. 4 depicts an embodiment for circularizing specific targets through the use of a “molecular clamp” to bring the two ends of the single stranded DNA into spatial proximity for ligation.

FIG. 5A, FIG. 5B, and FIG. 5C depict three different ways to prime a rolling circle amplification (RCA) reaction. FIG. 5A shows the use of target specific primers, e.g. the particular target genes or target sequences of interest. This generally results in only target sequences being amplified. FIG. 5B depicts the use of random primers to perform whole genome amplification (WGA), which will generally amplify all sample sequences, which then are bioinformatically sorted out during processing. FIG. 5C depicts the use of adapter primers when adapters are used, also resulting in general non-target-specific amplification.

FIG. 6 shows a PCR method in accordance with an embodiment that promotes sequencing of circular polynucleotides or strands containing at least two copies of a target nucleic acid sequence, using a pair of primers that are oriented away from one another when aligned within a monomer of the target sequence (also referred to as “back to back,” e.g. oriented in two directions but not on the ends of the domain to be amplified). In some embodiments, these primer sets are used after concatamers are formed to promote amplicons to be higher multimers, e.g. dimers, trimers, etc., of the target sequence. Optionally, the method can further include a size selection to remove amplicons that are smaller than dimers.

FIG. 7A, FIG. 7B, FIG. 7C, and FIG. 7D depict an embodiment in which back to back (B2B) primers are used with a “touch up” PCR step, such that amplification of short products (such as monomers) are less favored. In this case, the primers have two domains; a first domain that hybridizes to the target sequence (grey or black arrow) and a second domain that is a “universal primer” binding domain (bent rectangles; also sometimes referred to as an adapter) which does not hybridize to the original target sequence. In some embodiments, the first rounds of PCR are done with a low temperature annealing step, such that gene specific sequences bind. The low temperature run results in PCR products of various lengths, including short products. After a low number of rounds, the annealing temperature is raised, such that hybridization of the entire primer, both domains, is favored; as depicted these are found at the ends of the templates, while internal binding is less stable. Shorter products are thus less favored at the higher temperature with both domains than at the lower temperature or only a single domain.

FIG. 8 is an illustration of a system according to an embodiment.

FIG. 9A, FIG. 9B, FIG. 9C, and FIG. 9D depict examples of results obtained in a digital PCR assay to detect the sequence variant EGFRL858R using methods according to the disclosure.

FIG. 10A, FIG. 10B, FIG. 10C, and FIG. 10D depict examples of results obtained in a digital PCR assay to detect the sequence variant EGFRG719S using methods according to the disclosure.

FIG. 11A, FIG. 11B, FIG. 11C, and FIG. 11D depict examples of results obtained in a digital PCR assay to detect the sequence variant EGFR T790M using methods according to the disclosure.

### DETAILED DESCRIPTION OF THE INVENTION

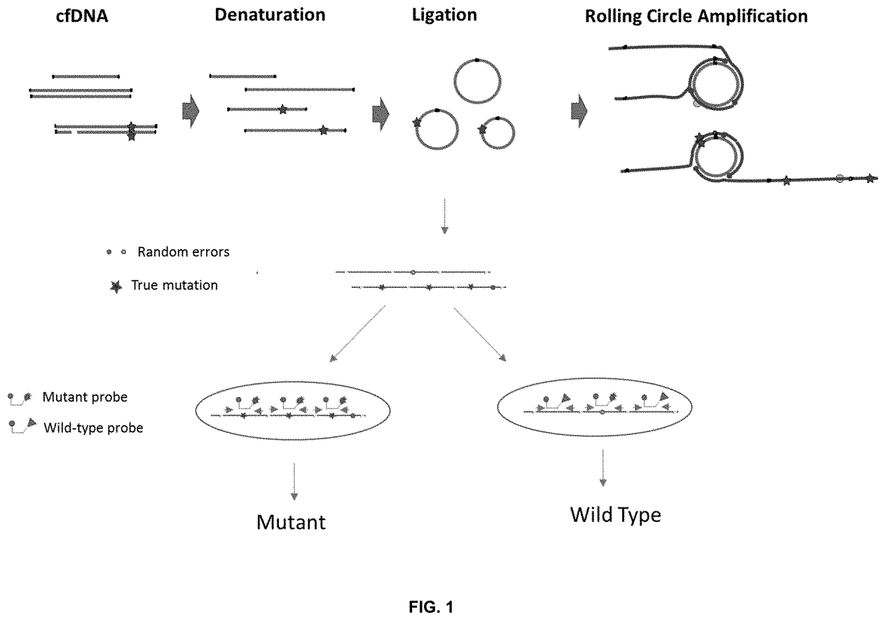

The systems and methods provided herein generally relate to digital PCR techniques and improvements thereon. In some cases, the systems and methods may be suitable for use on a nucleic acid sample comprising a small amount of starting material (e.g., cell-free DNA). In some cases, the systems and methods may provide improvements on traditional digital PCR techniques by reducing the number of false positive calls in a digital PCR assay. In some cases, the systems and methods may provide improvements on traditional digital PCR techniques by increasing the accuracy of a sequence variant call in a digital PCR assay. FIG. 1 depicts an example methodology for digital PCR assay according to embodiments of the disclosure. Generally, the methods may involve circularizing individual polynucleotides in a nucleic acid sample and amplifying the circularized polynucleotides to generate a plurality of concatemers. In some cases, the concatemers each contain a plurality of sequence repeats. In some cases, at least one of the plurality of concatemers may comprise a target sequence, and the target sequence may be repeated in the concatemer a plurality of times. In some cases, the target sequence may comprise a sequence variant. In some cases, the target sequence may contain an error that was introduced into the target sequence by an amplification step. In some cases, the methods may be used to distinguish between random errors and true mutations in a target sequence. As shown in FIG. 1, the plurality of concatemers may be partitioned into a plurality of partitions. In some cases, the plurality of concatemers may be partitioned into a plurality of partitions such that, on average, no more than one concatemer comprising a target sequence is present in an individual partition. The methods may further comprise hybridizing probes to the target sequence. In some cases, the probes may include a wild-type probe that is capable of binding to a wild-type sequence in the target sequence and producing a first signal (wild-type signal). In some cases, the probes may include a mutant probe that is capable of binding to a mutant sequence (e.g., containing the sequence variant) in the target sequence and producing a second signal (mutant signal). Without wishing to be bound by theory, if the starting polynucleotide contains a true mutation, it is expected that each target sequence in the plurality of sequence repeats would contain the sequence variant. In contrast, if the mutation was due to random error during an amplification step, it is expected that most of the target sequences in the plurality of sequence repeats would contain the wild-type sequence, with a small amount (1 or more) of the target sequences containing the error. Individual partitions may be interrogated and those that have a true mutation would be expected to generate a mutant signal (but not a wild-type signal), whereas individual partitions that have a random error may be expected to generate both a mutant and a wild-type signal.

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction.

In general, a “nucleotide probe,” “probe,” or “tag oligonucleotide” refers to a polynucleotide used for detecting or identifying its corresponding target polynucleotide in a hybridization reaction by hybridization with a corresponding target sequence. Thus, a nucleotide probe is hybridizable to one or more target polynucleotides. Tag oligonucleotides can be perfectly complementary to one or more target polynucleotides in a sample, or contain one or more nucleotides that are not complemented by a corresponding nucleotide in the one or more target polynucleotides in a sample.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is complementary to a first sequence is referred to as the “complement” of the first sequence. The term “hybridizable” as applied to a polynucleotide refers to the ability of the polynucleotide to form a complex that is stabilized via hydrogen bonding between the bases of the nucleotide residues in a hybridization reaction.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment may be assessed using any suitable parameters of a chosen algorithm, including default parameters.

In general, “stringent conditions” for hybridization refer to conditions under which a nucleic acid having complementarity to a target sequence predominantly hybridizes with a target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

In one aspect, the disclosure provides a method of identifying a sequence variant, such as in a nucleic acid sample. In some embodiments the method comprises: a) circularizing a plurality of polynucleotides to form a plurality of circularized polynucleotides; b) amplifying the plurality of circularized polynucleotides to generate a plurality of concatemers, each comprising a plurality of sequence repeats; partitioning the plurality of concatemers into a plurality of partitions, such that, on average, no more than one concatemer comprising a target sequence is present in an individual partition, wherein an individual partition of the plurality of partitions contains at least one of a first probe and a second probe, wherein the first probe binds to the target sequence that lacks the sequence variant and produces a first signal, and the second probe binds to the target sequence that contains the sequence variant and produces a second signal; c) detecting the first signal and the second signal from the individual partition; and d) identifying the sequence variant as present in the target sequence only when a level of the second signal exceeds that of a threshold level indicative of one copy of the target sequence, and a level of the first signal is below that of a threshold level indicative of one copy of the target sequence. In some cases, the method further comprises identifying the sequence variant as absent when a level of the first signal exceeds that of a threshold level indicative of one copy of the target sequence, and a level of the second signal is no greater than a threshold level indicative of one copy of the target sequence.

In general, the term “sequence variant” refers to any variation in sequence relative to one or more reference sequences. Typically, the sequence variant occurs with a lower frequency than the reference sequence for a given population of individuals for whom the reference sequence is known. For example, a particular bacterial genus may have a consensus reference sequence for the 16S rRNA gene, but individual species within that genus may have one or more sequence variants within the gene (or a portion thereof) that are useful in identifying that species in a population of bacteria. As a further example, sequences for multiple individuals of the same species (or multiple sequencing reads for the same individual) may produce a consensus sequence when optimally aligned, and sequence variants with respect to that consensus may be used to identify mutants in the population indicative of dangerous contamination. In general, a “consensus sequence” refers to a nucleotide sequence that reflects the most common choice of base at each position in the sequence where the series of related nucleic acids has been subjected to intensive mathematical and/or sequence analysis, such as optimal sequence alignment according to any of a variety of sequence alignment algorithms. A variety of alignment algorithms are available, some of which are described herein. In some embodiments, the reference sequence is a single known reference sequence, such as the genomic sequence of a single individual. In some embodiments, the reference sequence is a consensus sequence formed by aligning multiple known sequences, such as the genomic sequence of multiple individuals serving as a reference population, or multiple sequencing reads of polynucleotides from the same individual. In some embodiments, the reference sequence is a consensus sequence formed by optimally aligning the sequences from a sample under analysis, such that a sequence variant represents a variation relative to corresponding sequences in the same sample. In some embodiments, the sequence variant occurs with a low frequency in the population (also referred to as a “rare” sequence variant). For example, the sequence variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some embodiments, the sequence variant occurs with a frequency of about or less than about 0.1%.

A sequence variant can be any variation with respect to a reference sequence. A sequence variation may consist of a change in, insertion of, or deletion of a single nucleotide, or of a plurality of nucleotides (e.g. 2, 3, 4, 5, 6, 7, 8, 9, 10, or more nucleotides). Where a sequence variant comprises two or more nucleotide differences, the nucleotides that are different may be contiguous with one another, or discontinuous. Non-limiting examples of types of sequence variants include single nucleotide polymorphisms (SNP), deletion/insertion polymorphisms (DIP), copy number variants (CNV), short tandem repeats (STR), simple sequence repeats (SSR), variable number of tandem repeats (VNTR), amplified fragment length polymorphisms (AFLP), retrotransposon-based insertion polymorphisms, sequence specific amplified polymorphism, and differences in epigenetic marks that can be detected as sequence variants (e.g. methylation differences).

Nucleic acid samples that may be subjected to methods described herein can be derived from any suitable source. In some embodiments, the samples used are environmental samples. Environmental sample may be from any environmental source, for example, naturally occurring or artificial atmosphere, water systems, soil, or any other sample of interest. In some embodiments, the environmental samples may be obtained from, for example, atmospheric pathogen collection systems, sub-surface sediments, groundwater, ancient water deep within the ground, plant root-soil interface of grassland, coastal water and sewage treatment plants.

Polynucleotides from a sample may be any of a variety of polynucleotides, including but not limited to, DNA, RNA, ribosomal RNA (rRNA), transfer RNA (tRNA), micro RNA (miRNA), messenger RNA (mRNA), cell-free DNA (cfDNA), circulating tumor DNA (ctDNA), fragments of any of these, or combinations of any two or more of these. In some embodiments, samples comprise DNA. In some embodiments, samples comprise genomic DNA. In some embodiments, samples may comprise a low amount of polynucleotides (<50 ng). In some embodiments, samples comprise mitochondrial DNA, chloroplast DNA, plasmid DNA, bacterial artificial chromosomes, yeast artificial chromosomes, oligonucleotide tags, or combinations thereof. In some embodiments, the samples comprise DNA generated by amplification, such as by primer extension reactions using any suitable combination of primers and a DNA polymerase, including but not limited to polymerase chain reaction (PCR), reverse transcription, and combinations thereof Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. In general, sample polynucleotides comprise any polynucleotide present in a sample, which may or may not include target polynucleotides. The polynucleotides may be single-stranded, double-stranded, or a combination of these. In some embodiments, polynucleotides subjected to a method of the disclosure are single-stranded polynucleotides, which may or may not be in the presence of double-stranded polynucleotides. In some embodiments, the polynucleotides are single-stranded DNA. Single-stranded DNA (ssDNA) may be ssDNA that is isolated in a single-stranded form, or DNA that is isolated in double-stranded form and subsequently made single-stranded for the purpose of one or more steps in a method of the disclosure.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, which may correspond to dead or damaged cells. The identity of such cells may be used to characterize the cells or population of cells from which they are derived, such as tumor cells (e.g. in cancer detection), fetal cells (e.g. in prenatal diagnostic), cells from transplanted tissue (e.g. in early detection of transplant failure), or members of a microbial community.

If a sample is treated to extract polynucleotides, such as from cells in a sample, a variety of extraction methods are available. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993), with or without the use of an automated nucleic acid extractor, e.g., the Model 341 DNA Extractor available from Applied Biosystems (Foster City, Calif.); (2) stationary phase adsorption methods (U.S. Pat. No. 5,234,809; Walsh et al., 1991); and (3) salt-induced nucleic acid precipitation methods (Miller et al., (1988), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724. If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic acid isolation step, purification of nucleic acids can be performed after any step in the disclosed methods, such as to remove excess or unwanted reagents, reactants, or products. A variety of methods for determining the amount and/or purity of nucleic acids in a sample are available, such as by absorbance (e.g. absorbance of light at 260 nm, 280 nm, and a ratio of these) and detection of a label (e.g. fluorescent dyes and intercalating agents, such as SYBR green, SYBR blue, DAPI, propidium iodine, Hoechst stain, SYBR gold, ethidium bromide).

Where desired, polynucleotides from a sample may be fragmented prior to further processing. Fragmentation may be accomplished by any of a variety of methods, including chemical, enzymatic, and mechanical fragmentation. In some embodiments, the fragments have an average or median length from about 10 to about 1,000 nucleotides in length, such as betweenl0-800, 10-500, 50-500, 90-200, or 50-150 nucleotides. In some embodiments, the fragments have an average or median length of about or less than about 100, 200, 300, 500, 600, 800, 1000, or 1500 nucleotides. In some embodiments, the fragments range from about 90-200 nucleotides, and/or have an average length of about 150 nucleotides. In some embodiments, the fragmentation is accomplished mechanically comprising subjecting sample polynucleotides to acoustic sonication. In some embodiments, the fragmentation comprises treating the sample polynucleotides with one or more enzymes under conditions suitable for the one or more enzymes to generate double-stranded nucleic acid breaks. Examples of enzymes useful in the generation of polynucleotide fragments include sequence specific and non-sequence specific nucleases. Non-limiting examples of nucleases include DNase I, Fragmentase, restriction endonucleases, variants thereof, and combinations thereof. For example, digestion with DNase I can induce random double-stranded breaks in DNA in the absence of Mg++ and in the presence of Mn++. In some embodiments, fragmentation comprises treating the sample polynucleotides with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample polynucleotides leaves overhangs having a predictable sequence. Fragmented polynucleotides may be subjected to a step of size selecting the fragments via standard methods such as column purification or isolation from an agarose gel.

According to some embodiments, polynucleotides among the plurality of polynucleotides from a sample are circularized. Circularization can include joining the 5′ end of a polynucleotide to the 3′ end of the same polynucleotide, to the 3′ end of another polynucleotide in the sample, or to the 3′ end of a polynucleotide from a different source (e.g. an artificial polynucleotide, such as an oligonucleotide adapter). In some embodiments, the 5′ end of a polynucleotide is joined to the 3′ end of the same polynucleotide (also referred to as “self-joining”). In some embodiments, conditions of the circularization reaction are selected to favor self-joining of polynucleotides within a particular range of lengths, so as to produce a population of circularized polynucleotides of a particular average length. For example, circularization reaction conditions may be selected to favor self-joining of polynucleotides shorter than about 5000, 2500, 1000, 750, 500, 400, 300, 200, 150, 100, 50, or fewer nucleotides in length. In some embodiments, fragments having lengths between 50-5000 nucleotides, 100-2500 nucleotides, or 150-500 nucleotides are favored, such that the average length of circularized polynucleotides falls within the respective range. In some embodiments, 80% or more of the circularized fragments are between 50-500 nucleotides in length, such as between 50-200 nucleotides in length. Reaction conditions that may be optimized include the length of time allotted for a joining reaction, the concentration of various reagents, and the concentration of polynucleotides to be joined. In some embodiments, a circularization reaction preserves the distribution of fragment lengths present in a sample prior to circularization. For example, one or more of the mean, median, mode, and standard deviation of fragment lengths in a sample before circularization and of circularized polynucleotides are within 75%, 80%, 85%, 90%, 95%, or more of one another.

Rather than preferentially forming self-joining circularization products, one or more adapter oligonucleotides may be used, such that the 5′ end and 3′ end of a polynucleotide in the sample are joined by way of one or more intervening adapter oligonucleotides to form a circular polynucleotide. For example, the 5′ end of a polynucleotide can be joined to the 3′ end of an adapter, and the 5′ end of the same adapter can be joined to the 3′ end of the same polynucleotide. An adapter oligonucleotide includes any oligonucleotide having a sequence, at least a portion of which is known, that can be joined to a sample polynucleotide. Adapter oligonucleotides can comprise DNA, RNA, nucleotide analogues, non-canonical nucleotides, labeled nucleotides, modified nucleotides, or combinations thereof. Adapter oligonucleotides can be single-stranded, double-stranded, or partial duplex. In general, a partial-duplex adapter comprises one or more single-stranded regions and one or more double-stranded regions. Double-stranded adapters can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. When two hybridized regions of an adapter are separated from one another by a non-hybridized region, a “bubble” structure results. Adapters of different kinds can be used in combination, such as adapters of different sequences. Different adapters can be joined to sample polynucleotides in sequential reactions or simultaneously. In some embodiments, identical adapters are added to both ends of a target polynucleotide. For example, first and second adapters can be added to the same reaction. Adapters can be manipulated prior to combining with sample polynucleotides. For example, terminal phosphates can be added or removed.

Where adapter oligonucleotides are used, the adapter oligonucleotides can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as flow cells as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. In some cases, the adapters may be used to purify those circles that contain the adapters, for example by using beads (particularly magnetic beads for ease of handling) that are coated with oligonucleotides comprising a complementary sequence to the adapter, that can “capture” the closed circles with the correct adapters by hybridization thereto, wash away those circles that do not contain the adapters and any unligated components, and then release the captured circles from the beads. In addition, in some cases, the complex of the hybridized capture probe and the target circle can be directly used to generate concatemers, such as by direct rolling circle amplification (RCA). In some embodiments, the adapters in the circles can also be used as a sequencing primer. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adapter oligonucleotide. A sequence element may be of any suitable length, such as about or less than about 3, 4, 5, 6, 7, 8, 9, 10, 15, 20, 25, 30, 35, 40, 45, 50 or more nucleotides in length. Adapter oligonucleotides can have any suitable length, at least sufficient to accommodate the one or more sequence elements of which they are comprised. In some embodiments, adapters are about or less than about 10, 15, 20, 25, 30, 35, 40, 45, 50, 55, 60, 65, 70, 75, 80, 90, 100, 200, or more nucleotides in length. In some embodiments, an adapter oligonucleotide is in the range of about 12 to 40 nucleotides in length, such as about 15 to 35 nucleotides in length.

In some embodiments, the adapter oligonucleotides joined to fragmented polynucleotides from one sample comprise one or more sequences common to all adapter oligonucleotides and a barcode that is unique to the adapters joined to polynucleotides of that particular sample, such that the barcode sequence can be used to distinguish polynucleotides originating from one sample or adapter joining reaction from polynucleotides originating from another sample or adapter joining reaction. In some embodiments, an adapter oligonucleotide comprises a 5′ overhang, a 3′ overhang, or both that is complementary to one or more target polynucleotide overhangs. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs of an adapter oligonucleotide may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some embodiments, an adapter overhang is complementary to a target polynucleotide overhang produced by restriction endonuclease digestion. In some embodiments, an adapter overhang consists of an adenine or a thymine.

A variety of methods for circularizing polynucleotides are available. In some embodiments, circularization comprises an enzymatic reaction, such as use of a ligase (e.g. an RNA or DNA ligase). A variety of ligases are available, including, but not limited to, Circligase™ (Epicentre; Madison, Wis.), RNA ligase, T4 RNA Ligase 1 (ssRNA Ligase, which works on both DNA and RNA). In addition, T4 DNA ligase can also ligate ssDNA if no dsDNA templates are present, although this is generally a slow reaction. Other non-limiting examples of ligases include NAD-dependent ligases including Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof Where self-joining is desired, the concentration of polynucleotides and enzyme can be adjusted to facilitate the formation of intramolecular circles rather than intermolecular structures. Reaction temperatures and times can be adjusted as well. In some embodiments, 60° C. is used to facilitate intramolecular circles. In some embodiments, reaction times are between 12-16 hours. Reaction conditions may be those specified by the manufacturer of the selected enzyme. In some embodiments, an exonuclease step can be included to digest any unligated nucleic acids after the circularization reaction. That is, closed circles do not contain a free 5′ or 3′ end, and thus the introduction of a 5′ or 3′ exonuclease will not digest the closed circles but will digest the unligated components. This may find particular use in multiplex systems.

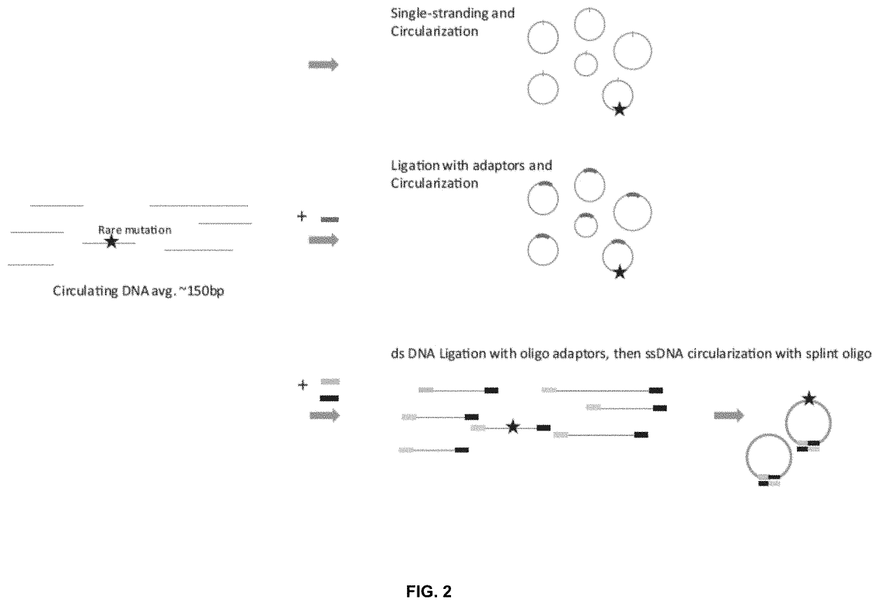

FIG. 2 illustrates three non-limiting examples of methods of circularized polynucleotides. At the top, the polynucleotides are circularized in the absence of adapters, while the middle scheme depicts the use of adapters, and the bottom scheme utilizes two adapters. Where two adapters are used, one can be joined to the 5′ end of the polynucleotide while the second adapter can be joined to the 3′ end of the same polynucleotide. In some embodiments, adapter ligation may comprise use of two different adapters along with a “splint” nucleic acid that is complementary to the two adapters to facilitate ligation. Forked or “Y” adapters may also be used. Where two adapters are used, polynucleotides having the same adapter at both ends may be removed in subsequent steps due to self-annealing.

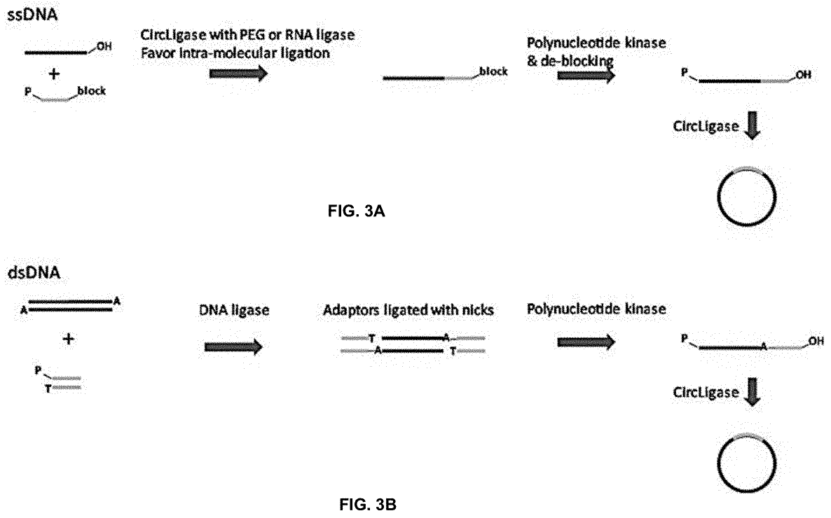

FIG. 3A and FIG. 3B illustrate further non-limiting example methods of circularizing polynucleotides, such as single-stranded DNA. The adapter can be asymmetrically added to either the 5′ or 3′ end of a polynucleotide. As shown in FIG. 3A, the single-stranded DNA (ssDNA) has a free hydroxyl group at the 3′ end, and the adapter has a blocked 3′ end such that in the presence of a ligase, a preferred reaction joins the 3′ end of the ssDNA to the 5′ end of the adapter. In this embodiment, it can be useful to use agents such as polyethylene glycols (PEGs) to drive the intermolecular ligation of a single ssDNA fragment and a single adapter, prior to an intramolecular ligation to form a circle. The reverse order of ends can also be done (blocked 3′, free 5′, etc.). Once the linear ligation is accomplished, the ligated pieces can be treated with an enzyme to remove the blocking moiety, such as through the use of a kinase or other suitable enzymes or chemistries. Once the blocking moiety is removed, the addition of a circularization enzyme, such as CircLigase, allows an intramolecular reaction to form the circularized polynucleotide. As shown in FIG. 3B, by using a double-stranded adapter with one strand having a 5′ or 3′ end blocked, a double stranded structure can be formed, which upon ligation produces a double-stranded fragment with nicks. The two strands can then be separated, the blocking moiety removed, and the single-stranded fragment circularized to form a circularized polynucleotide.

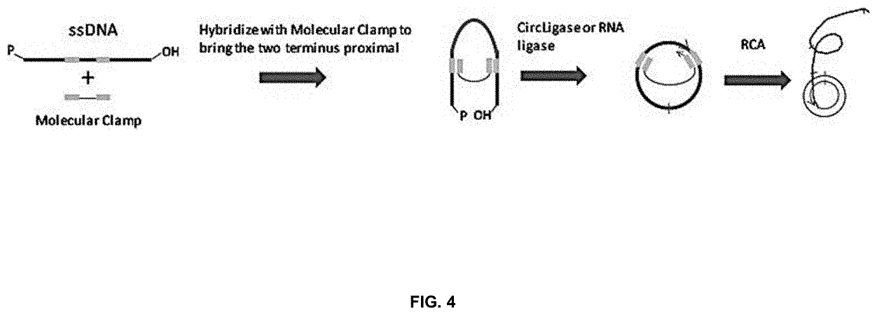

In some embodiments, molecular clamps are used to bring two ends of a polynucleotide (e.g. a single-stranded DNA) together in order to enhance the rate of intramolecular circularization. An example illustration of one such process is illustrated in FIG. 4. This can be done with or without adapters. The use of molecular clamps may be particularly useful in cases where the average polynucleotide fragment is greater than about 100 nucleotides in length. In some embodiments, the molecular clamp probe comprises three domains: a first domain, an intervening domain, and a second domain. The first and second domains will hybridize to first to corresponding sequences in a target polynucleotide via sequence complementarity. The intervening domain of the molecular clamp probe does not significantly hybridize with the target sequence. The hybridization of the clamp with the target polynucleotide thus brings the two ends of the target sequence into closer proximity, which facilitates the intramolecular circularization of the target sequence in the presence of a circularization enzyme. In some embodiments, this is additionally useful as the molecular clamp can serve as an amplification primer as well.

After circularization, reaction products may be purified prior to amplification or sequencing to increase the relative concentration or purity of circularized polynucleotides available for participating in subsequent steps (e.g., by isolation of circular polynucleotides or removal of one or more other molecules in the reaction). For example, a circularization reaction or components thereof may be treated to remove single-stranded (non-circularized) polynucleotides, such as by treatment with an exonuclease. As a further example, a circularization reaction or portion thereof may be subjected to size exclusion chromatography, whereby small reagents are retained and discarded (e.g. unreacted adapters), or circularization products are retained and released in a separate volume. A variety of kits for cleaning up ligation reactions are available, such as kits provided by Zymo oligo purification kits made by Zymo Research. In some embodiments, purification comprises treatment to remove or degrade ligase used in the circularization reaction, and/or to purify circularized polynucleotides away from such ligase. In some embodiments, treatment to degrade ligase comprises treatment with a protease, such as proteinase K. Proteinase K treatment may follow manufacturer protocols, or standard protocols (e.g. as provided in Sambrook and Green, Molecular Cloning: A Laboratory Manual, 4th Edition (2012)). Protease treatment may also be followed by extraction and precipitation. In one example, circularized polynucleotides are purified by proteinase K (Qiagen) treatment in the presence of 0.1% SDS and 20 mM EDTA, extracted with 1:1 phenol/chloroform and chloroform, and precipitated with ethanol or isopropanol. In some embodiments, precipitation is in ethanol.

In some cases, an amplification reaction may be performed on the circular polynucleotides (e.g., preamplification) prior to performing a digital polymerase chain reaction (dPCR) according to the methods provided herein. In general, “amplification” refers to a process by which one or more copies are made of a target polynucleotide or a portion thereof. A variety of methods of amplifying polynucleotides (e.g. DNA and/or RNA) are available. Amplification may be linear, exponential, or involve both linear and exponential phases in a multi-phase amplification process. Amplification methods may involve changes in temperature, such as a heat denaturation step, or may be isothermal processes that do not require heat denaturation. The polymerase chain reaction (PCR) uses multiple cycles of denaturation, annealing of primer pairs to opposite strands, and primer extension to exponentially increase copy numbers of the target sequence. Denaturation of annealed nucleic acid strands may be achieved by the application of heat, increasing local metal ion concentrations (e.g. U.S. Pat. No. 6,277,605), ultrasound radiation (e.g. WO/2000/049176), application of voltage (e.g. U.S. Pat. Nos. 5,527,670, 6,033,850, 5,939,291, and 6,333,157), and application of an electromagnetic field in combination with primers bound to a magnetically-responsive material (e.g. U.S. Pat. No. 5,545,540). In a variation called RT-PCR, reverse transcriptase (RT) is used to make a complementary DNA (cDNA) from RNA, and the cDNA is then amplified by PCR to produce multiple copies of DNA (e.g. U.S. Pat. Nos. 5,322,770 and 5,310,652). One example of an isothermal amplification method is strand displacement amplification, commonly referred to as SDA, which uses cycles of annealing pairs of primer sequences to opposite strands of a target sequence, primer extension in the presence of a dNTP to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product (e.g. U.S. Pat. Nos. 5,270,184 and 5,455,166). Thermophilic SDA (tSDA) uses thermophilic endonucleases and polymerases at higher temperatures in essentially the same method (European Pat. No. 0 684 315). Other amplification methods include rolling circle amplification (RCA) (e.g., Lizardi, “Rolling Circle Replication Reporter Systems,” U.S. Pat. No. 5,854,033); helicase dependent amplification (HDA) (e.g., Kong et al., “Helicase Dependent Amplification Nucleic Acids,” U.S. Pat. Appin. Pub. No. US 2004-0058378 A1); and loop-mediated isothermal amplification (LAMP) (e.g., Notomi et al., “Process for Synthesizing Nucleic Acid,” U.S. Pat. No. 6,410,278). In some cases, isothermal amplification utilizes transcription by an RNA polymerase from a promoter sequence, such as may be incorporated into an oligonucleotide primer. Transcription-based amplification methods include nucleic acid sequence based amplification, also referred to as NASBA (e.g. U.S. Pat. No. 5,130,238); methods which rely on the use of an RNA replicase to amplify the probe molecule itself, commonly referred to as Q13 replicase (e.g., Lizardi, P. et al. (1988) BioTechnol. 6, 1197-1202); self-sustained sequence replication (e.g., Guatelli, J. et al. (1990) Proc. Natl. Acad. Sci. USA 87, 1874-1878; Landgren (1993) Trends in Genetics 9, 199-202; and HELEN H. LEE et al., NUCLEIC ACID AMPLIFICATION T ECHNOLOGIES (1997)); and methods for generating additional transcription templates (e.g. U.S. Pat. Nos. 5,480,784 and 5,399,491). Further methods of isothermal nucleic acid amplification include the use of primers containing non-canonical nucleotides (e.g. uracil or RNA nucleotides) in combination with an enzyme that cleaves nucleic acids at the non-canonical nucleotides (e.g. DNA glycosylase or RNaseH) to expose binding sites for additional primers (e.g. U.S. Pat. Nos. 6,251,639, 6,946,251, and 7,824,890). Isothermal amplification processes can be linear or exponential.

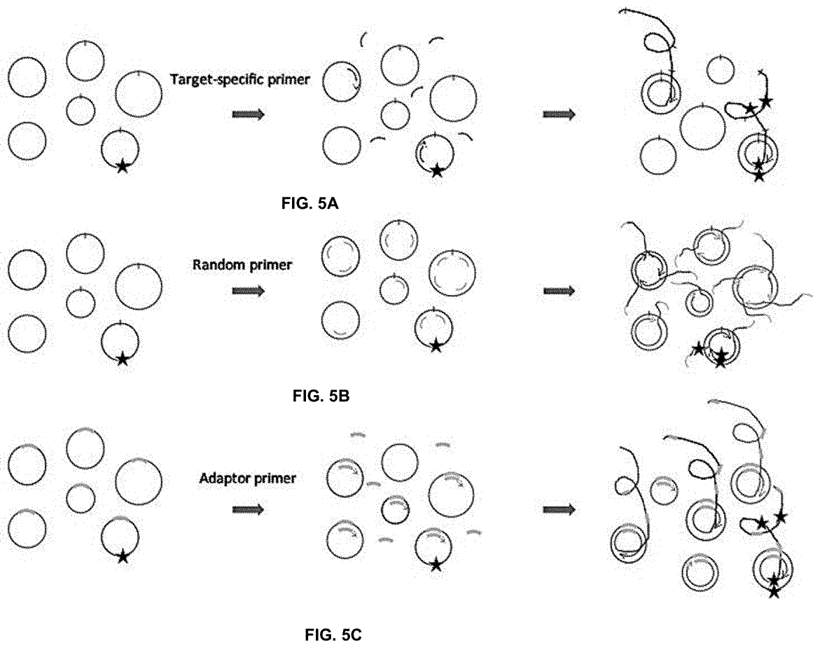

In some embodiments, amplification comprises rolling circle amplification (RCA). A typical RCA reaction mixture comprises one or more primers, a polymerase, and dNTPs, and produces concatemers. Typically, the polymerase in an RCA reaction is a polymerase having strand-displacement activity. A variety of such polymerases are available, non-limiting examples of which include exonuclease minus DNA Polymerase I large (Klenow) Fragment, Phi29 DNA polymerase, Taq DNA Polymerase and the like. In general, a concatemer is a polynucleotide amplification product comprising two or more copies of a target sequence from a template polynucleotide (e.g. about or more than about 2, 3, 4, 5, 6, 7, 8, 9 ,10, or more copies of the target sequence; in some embodiments, about or more than about 2 copies). Amplification primers may be of any suitable length, such as about or at least about 5, 10, 15, 20, 25, 30, 35, 40, 45, 50, 55, 60, 65, 70, 75, 80, 90, 100, or more nucleotides, any portion or all of which may be complementary to the corresponding target sequence to which the primer hybridizes (e.g. about, or at least about 5, 10, 15, 20, 25, 30, 35, 40, 45, 50, or more nucleotides). FIG. 5A, FIG. 5B, and FIG. 5C depict three non-limiting examples of suitable primers. FIG. 5A shows the use of no adapters and a target specific primer, which can be used for the detection of the presence or absence of a sequence variant within specific target sequences. In some embodiments, multiple target-specific primers for a plurality of targets are used in the same reaction. For example, target-specific primers for about or at least about 10, 50, 100, 150, 200, 250, 300, 400, 500, 1000, 2500, 5000, 10000, 15000, or more different target sequences may be used in a single amplification reaction in order to amplify a corresponding number of target sequences (if present) in parallel. Multiple target sequences may correspond to different portions of the same gene, different genes, or non-gene sequences. Where multiple primers target multiple target sequences in a single gene, primers may be spaced along the gene sequence (e.g. spaced apart by about or at least about 50 nucleotides, every 50-150 nucleotides, or every 50-100 nucleotides) in order to cover all or a specified portion of a target gene. FIG. 5C illustrates use of a primer that hybridizes to an adapter sequence (which in some cases may be an adapter oligonucleotide itself).

FIG. 5B illustrates an example of amplification by random primers. In general, a random primer comprises one or more random or near-random sequences (e.g., one or more nucleotides selected at random from a set of two or more different nucleotides at one or more positions, with each of the different nucleotides selected at one or more positions represented in a pool of adapters comprising the random sequence). In this way, polynucleotides (e.g., all or substantially all circularized polynucleotides) can be amplified in a sequence non-specific fashion. Such procedures may be referred to as “whole genome amplification” (WGA); however, typical WGA protocols (which do not involve a circularization step) do not efficiently amplify short polynucleotides, such as polynucleotide fragments contemplated by the present disclosure. For further illustrative discussion of WGA procedures, see for example Li et al (2006) J Mol. Diagn. 8(1):22-30.

Where circularized polynucleotides are amplified prior to dPCR, amplified products may be subjected to dPCR directly without enrichment, or subsequent to one or more enrichment steps. Enrichment may comprise purifying one or more reaction components, such as by retention of amplification products or removal of one or more reagents. For example, amplification products may be purified by hybridization to a plurality of probes attached to a substrate, followed by release of captured polynucleotides, such as by a washing step. Alternatively, amplification products can be labeled with a member of a binding pair followed by binding to the other member of the binding pair attached to a substrate, and washing to release the amplification product. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. In some embodiments, the substrate is in the form of a bead or other small, discrete particle, which may be a magnetic or paramagnetic bead to facilitate isolation through application of a magnetic field. In general, “binding pair” refers to one of a first and a second moiety, wherein the first and the second moiety have a specific binding affinity for each other. Suitable binding pairs include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine); biotin/avidin (or biotin/streptavidin); calmodulin binding protein (CBP)/calmodulin; hormone/hormone receptor; lectin/carbohydrate; peptide/cell membrane receptor; protein A/antibody; hapten/antihapten; enzyme/cofactor; and enzyme/substrate.

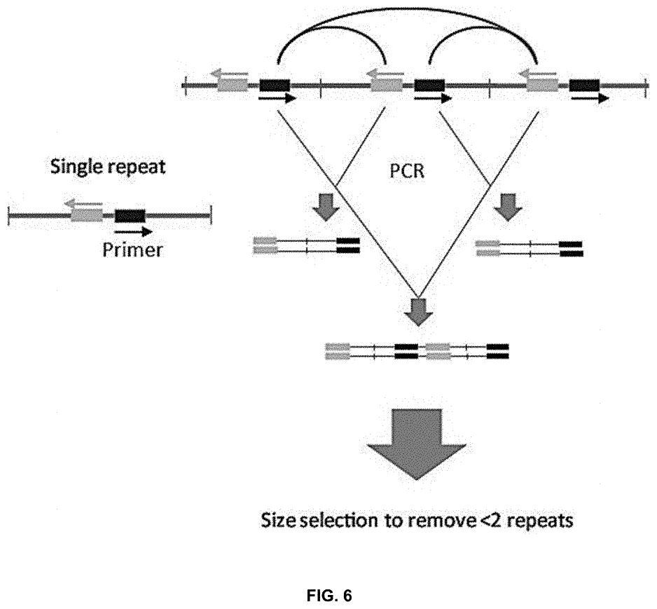

In some embodiments, enrichment following amplification of circularized polynucleotides comprises one or more additional amplification reactions. In some embodiments, enrichment comprises amplifying a target sequence comprising sequence A and sequence B (oriented in a 5′ to 3′ direction) in an amplification reaction mixture comprising (a) the amplified polynucleotide; (b) a first primer comprising sequence A′, wherein the first primer specifically hybridizes to sequence A of the target sequence via sequence complementarity between sequence A and sequence A′; (c) a second primer comprising sequence B, wherein the second primer specifically hybridizes to sequence B′ present in a complementary polynucleotide comprising a complement of the target sequence via sequence complementarity between B and B′; and (d) a polymerase that extends the first primer and the second primer to produce amplified polynucleotides; wherein the distance between the 5′ end of sequence A and the 3′ end of sequence B of the target sequence is 75nt or less. FIG. 6 illustrates an example arrangement of the first and second primer with respect to a target sequence in the context of a single repeat (which will typically not be amplified unless circular) and concatemers comprising multiple copies of the target sequence. Given the orientation of the primers with respect to a monomer of the target sequence, this arrangement may be referred to as “back to back” (B2B) or “inverted” primers. Amplification with B2B primers facilitates enrichment of circular and/or concatemeric amplification products. Moreover, this orientation combined with a relatively smaller footprint (total distance spanned by a pair of primers) permits amplification of a wider variety of fragmentation events around a target sequence, as a junction is less likely to occur between primers than in the arrangement of primers found in a typical amplification reaction (facing one another, spanning a target sequence). In some embodiments, the distance between the 5′ end of sequence A and the 3′ end of sequence B is about or less than about 200, 150, 100, 75, 50, 40, 30, 25, 20, 15, or fewer nucleotides. In some embodiments, sequence A is the complement of sequence B. In some embodiments, multiple pairs of B2B primers directed to a plurality of different target sequences are used in the same reaction to amplify a plurality of different target sequences in parallel (e.g. about or at least about 10, 50, 100, 150, 200, 250, 300, 400, 500, 1000, 2500, 5000, 10000, 15000, or more different target sequences). Primers can be of any suitable length, such as described elsewhere herein. Amplification may comprise any suitable amplification reaction under appropriate conditions, such as an amplification reaction described herein. In some embodiments, amplification is a polymerase chain reaction.

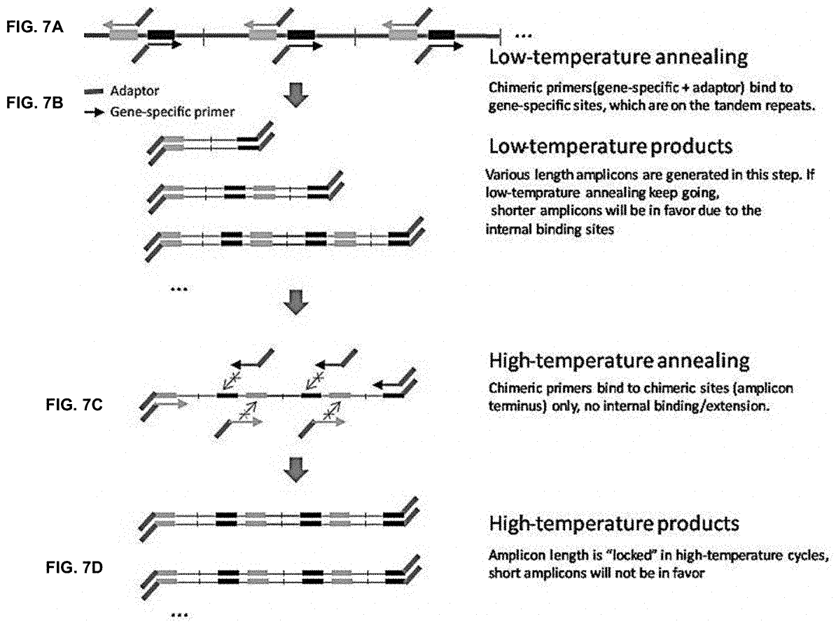

In some embodiments, B2B primers comprise at least two sequence elements, a first element that hybridizes to a target sequence via sequence complementarity, and a 5′ “tail” that does not hybridize to the target sequence during a first amplification phase at a first hybridization temperature during which the first element hybridizes (e.g. due to lack of sequence complementarity between the tail and the portion of the target sequence immediately 3′ with respect to where the first element binds). For example, the first primer comprises sequence C 5′ with respect to sequence A′, the second primer comprises sequence D 5′ with respect to sequence B, and neither sequence C nor sequence D hybridize to the plurality of concatemers during a first amplification phase at a first hybridization temperature. In some embodiments in which such tailed primers are used, amplification can comprise a first phase and a second phase; the first phase comprises a hybridization step at a first temperature, during which the first and second primers hybridize to the concatemers (or circularized polynucleotides) and primer extension; and the second phase comprises a hybridization step at a second temperature that is higher than the first temperature, during which the first and second primers hybridize to amplification products comprising extended first or second primers, or complements thereof, and primer extension. The higher temperature favors hybridization between along the first element and tail element of the primer in primer extension products over shorter fragments formed by hybridization between only the first element in a primer and an internal target sequence within a concatemer. Accordingly, the two-phase amplification may be used to reduce the extent to which short amplification products might otherwise be favored, thereby maintaining a relatively higher proportion of amplification products having two or more copies of a target sequence. For example, after 5 cycles (e.g. at least 5, 6, 7, 8, 9, 10, 15, 20, or more cycles) of hybridization at the second temperature and primer extension, at least 5% (e.g. at least 5%, 6%, 7%, 8%, 9%, 10%, 15%, 20%, 25%, 30%, or more) of amplified polynucleotides in the reaction mixture comprise two or more copies of the target sequence. An illustration of embodiments in accordance with this two-phase, tailed B2B primer amplification process is presented in FIG. 7A, FIG. 7B, FIG. 7C, and FIG. 7D.

In some embodiments, enrichment comprises amplification under conditions that are skewed to increase the length of amplicons from concatemers. For example, the primer concentration can be lowered, such that not every priming site will hybridize a primer, thus making the PCR products longer. Similarly, decreasing the primer hybridization time during the cycles will similarly allow fewer primers to hybridize, thus also making the average PCR amplicon size increase. Furthermore, increasing the temperature and/or extension time of the cycles will similarly increase the average length of the PCR amplicons. Any combination of these techniques can be used.

In some embodiments, amplification products are treated to filter the resulting amplicons on the basis of size to reduce and/or eliminate the number of monomers in a mixture comprising concatemers. This can be done using a variety of available techniques, including, but not limited to, fragment excision from gels and gel filtration (e.g. to enrich for fragments larger than about 300, 400, 500, or more nucleotides in length); as well as SPRI beads (Agencourt AMPure XP) for size selection by fine-tuning the binding buffer concentration. For example, the use of 0.6× binding buffer during mixing with DNA fragments may be used to preferentially bind DNA fragments larger than about 500 base pairs (bp).

In some aspects, the methods may further comprise partitioning the plurality of concatemers into a plurality of partitions. “Partitioning” generally refers to the process of spatially separating a mixture containing a plurality of molecules into at least one partition. A “partition” as used herein may refer to any container or vessel for spatially separating a plurality of molecules. In some cases, a partition may be a well, such as, e.g., a well on a microplate. In other cases, a partition may be a droplet, such as, e.g., a droplet used in a droplet digital PCR (ddPCR) method. Droplets may include water-in-oil emulsion droplets or oil-in-water emulsion droplets. Non-limiting examples of droplet-based PCR systems that may be used in accordance with the methods provided herein include those systems commercially available from Bio-Rad, Raindance Technologies, among others. Generally, the number of individual concatemers present in an individual partition after partitioning depends on the concentration of the concatemers in the mixture, and the number of partitions the mixture is partitioned into. In some cases, the method involves partitioning the plurality of concatemers into a plurality of partitions, such that, on average, any individual partition comprises no more than one concatemer having a target sequence. In such cases, the individual partition may comprise a concatemer comprising a plurality of sequence repeats, wherein each of the plurality of sequence repeats comprises the target sequence. Thus, an individual partition may comprise a plurality of target sequences arranged in tandem repeats on the same concatemer molecule. In some cases, the individual partition may also comprise one or more concatemers that do not comprise a target sequence. In some cases, an individual partition comprises, on average, no more than 10, 9, 8, 7, 6, 5, 4, 3, 2, or 1 concatemers. In some cases, a plurality of individual partitions may comprise zero concatemers.

In some aspects, an individual partition may comprise one or more probes for detecting a presence or absence of a sequence variant. In some cases, the one or more probes comprises a wild-type probe that binds to a wild-type target sequence (i.e., a target sequence that lacks the sequence variant). The wild-type probe may comprise an oligonucleotide sequence that is complementary to and capable of hybridizing to a wild-type target sequence. In some cases, the wild-type probe may comprise an oligonucleotide sequence that hybridizes to a region of the target sequence that contains a wild-type nucleotide at the nucleotide position under investigation. In some cases, the one or more probes comprises a mutant probe that binds to a mutant target sequence (i.e., a target sequence that contains a sequence variant). The mutant probe may comprise an oligonucleotide sequence that is complementary to and capable of hybridizing to a mutant target sequence. In some cases, the wild-type probe and the mutant probe may be hybridized to a target sequence under stringent conditions, such that the wild-type probe will only bind to a wild-type target sequence and the mutant probe will only bind to the mutant probe. In some cases, an individual partition may contain a wild-type probe, a mutant probe, or both.

In some aspects, the wild-type probe comprises a first detectable label that produces a first signal when the wild-type target sequence is present. In some aspects, the mutant probe comprises a second detectable label that produces a second signal when the mutant target sequence is present. In some cases, the first detectable label and the second detectable label are different, such that they produce different signals which can be distinguished. The first detectable label, the second detectable label, or both may be any type of detectable label, including, without limitation a fluorophore, an enzyme, a quencher, an enzyme inhibitor, a radioactive label, one member of a binding pair, or any combination thereof. In some cases, the first and/or second detectable labels are fluorescent molecules, e.g., fluorophores. Non-limiting examples of fluorophores may include: fluorescein (FITC) and fluorescein derivatives such as FAM, VIC, and JOE, 5-(2′-aminoethyl)aminonaphthalene-l-sulphonic acid (EDANS), coumarin and coumarin derivatives, Lucifer yellow, NED, Texas red, tetramethylrhodamine, tetrachloro-6-carboxyfluoroscein, 5 carboxyrhodamine, cyanine dye, Alexa Fluor 350, Alexa Fluor 647, Oregon Green, Alexa Fluor 405, Alexa Fluor 680, Alexa Fluor 488, Alexa Fluor 750, Cy3, Alexa Fluor 532, Pacific Blue, Pacific Orange, Alexa Fluor 546, Tetramethylrhodamine (TRITC), Alexa Fluor 555, BODIPY FL, Texas Red, Alexa Fluor 568, Pacific Green, Cy5, Alexa Fluor 594, Super Bright 436, Super Bright 600, Super Bright 645, Super Bright 702, DAPI, SYTOX Green, SYTO 9, TO-PRO-3, Propidium Iodide, Qdot 525, Qdot 565, Qdot 605, Qdot 655, Qdot 705, Qdot 800, R-Phycoerythrin (R-PE), Allophycocyanin (APC), cyan fluorescent protein (CFP) and derivatives thereof, green fluorescent protein (GFP) and derivatives thereof, red fluorescent protein (RFP) and derivatives thereof, and the like. Any fluorophore with an excitation wavelength of between about 300 nm and about 900 nm is envisioned herein.

In some aspects, the method may further comprise performing a reaction on or within the plurality of partitions. In some cases, the methods may further comprise performing a Taqman® PCR assay on the plurality of partitions. In such cases, the wild-type probe and the mutant probe may be Taqman® _{probes. Taqman® PCR assays and probes are known in the art. The} 5′ end of the wild-type probe and the mutant probe may be conjugated to different fluorescent labels (e.g., VIC, FAM). In addition, the 3′ end of the wild-type probe and the mutant probe may be conjugated to a quencher. When in close proximity to the fluorescent label (e.g., when the quencher and the fluorescent label are conjugated to opposite ends of the probe), the quencher may quench the signal from the fluorescent label. Individual partitions may further include a forward and a reverse primer which hybridize to a sequence on the concatemer that flanks the target sequence. In some cases, the forward and reverse primers may be unlabeled. The plurality of partitions may be incubated under conditions such that the forward primer, the reverse primer, and the mutant and/or wild-type probes hybridize to their complementary sequence, if present on the concatemer. In some cases, the method further comprises incubating the plurality of partitions in the presence of a polymerase and under conditions such that the polymerase synthesizes new oligonucleotide strands by extending the forward and reverse primers along the template molecule. The polymerase may contain endogenous 5′ nuclease activity such that when the polymerase reaches the labeled probe, it may cleave the probe, thereby separating the fluorescent label and the quencher. The fluorescent label may then generate a signal that can be detected. In some cases, multiple cycles of Taqman PCR are performed on the plurality of partitions, such that with each cycle, the intensity of the fluorescent signal increases in proportion to the amount of amplicon synthesized.

In some cases, the methods may further comprise performing an assay other than a Taqman® PCR assay on the plurality of partitions. Non-Taqman® based approaches may include, without limitation, SYBR® chemistry detection, Evagreen®—based detection, FAM-based detection, and the like.

In further aspects, the methods may comprise detecting a level of the first signal and the second signal from individual partitions. The detecting may involve any method for detecting a signal, and should be selected based on the type of detectable label present on the probes. In cases in which fluorescent probes are used, the method may involve illuminating the plurality of partitions with a fluorescent light source (e.g., a light-emitting diode (LED)), and measuring an optical signal generated therefrom. It is to be understood that the wavelength of light provided by the light source should be selected based on the excitation wavelength of the detectable label, and can readily be selected by a person of skill in the art.

In further aspects, the methods may comprise identifying the presence or absence of a sequence variant. In some cases, identifying the presence or absence of a sequence variant may comprise measuring an intensity level of a first signal corresponding to the presence of a wild-type sequence, and an intensity level of a second signal corresponding to a mutant sequence. The method may further comprise comparing the intensity level of a first signal and the intensity level of a second signal to a threshold level. In some cases, the threshold level represents a cut-off value for which signals that exceed the threshold level are determined to be present or positive, and signals that are below the threshold level are determined to be absent or negative. In some cases, the threshold level is determined by a user of the assay. In some cases, the threshold level is indicative of the presence of one copy of the target sequence. Put another way, a signal that exceeds the threshold level may be determined to contain at least one copy of the target sequence, and a signal that is below the threshold level may be determined to contain less than one copy of the target sequence.

In some cases, the sequence variant is identified as present in said target sequence only when a level of said mutant signal exceeds that of a threshold level, and a level of said first signal is below that of a threshold level. For example, if the sequence variant is present in the original sample, it will be represented multiple times in a single concatemer molecule. In such cases, the mutant probe may bind to the target sequence containing the sequence variant, but the wild-type probe may be unable to bind to the target sequence. Thus, individual partitions that contain the sequence variant may generate a signal from the mutant probe, but not from the wild-type probe.

In some cases, the sequence variant is identified as absent (i.e., the target sequence is a wild-type sequence) when a level of the wild-type signal exceeds that of a threshold level and a level of said mutant signal is below that of a threshold level. For example, if the sequence variant is absent in the original polynucleotide molecule, it may be absent in every sequence repeat of a single concatemer molecule. In such cases, the wild-type probe may bind to the target sequence that lacks the sequence variant, but the mutant probe may be unable to bind to the target sequence. Thus, individual partitions that contain the wild-type sequence may generate a signal from the wild-type probe, but not from the mutant probe.

In some cases, the methods may be used to identify a false positive. In one such embodiment, a false positive is identified when both a level of the wild-type signal exceeds that of a threshold level and a level of the mutant signal exceeds that of a threshold level. For example, random errors may be introduced into the target sequence during, e.g., amplification. In some cases, the target sequence may be a wild-type sequence, but an error may be introduced during rolling circle amplification that generates a mutation in at least one of the tandem repeats of the concatemer. In such cases, an individual partition may include a concatemer molecule comprising tandem repeats of the target sequence, with most of the repeats containing the wild-type sequence, but at least one of the repeats containing a sequence variant (e.g., due to random error). In such cases, the wild-type probe may bind to the wild-type target sequence, and the mutant probe may bind to the mutant target sequence, thereby generating both a wild-type signal and a mutant signal in the same partition. In some aspects, the method may identify such partitions as containing a false positive when both the wild-type signal and the mutant signal are present.

In further aspects, the methods may involve outputting a result based on the identifying steps described above. For example, the methods may involve generating a report displaying or reporting the results of the identifying steps. In some cases, partitions identified as containing a false positive may be excluded from the report. In other cases, partitions identified as containing a false positive may be flagged or reported as containing a false positive.

In another aspect, the disclosure provides a method for reducing error in a digital polymerase chain reaction. In some cases, the method may be performed on a nucleic acid sample comprising less than about 50 ng of polynucleotides, and further comprising: a) circularizing individual polynucleotides in the nucleic acid sample to generate a plurality of circularized polynucleotides; b) amplifying the plurality of circularized polynucleotides to form a plurality of concatemers, each comprising a plurality of sequence repeats; c) partitioning the plurality of concatemers into a plurality of partitions, such that, on average, no more than one concatemer comprising a target sequence is present in an individual partition, wherein an individual partition of the plurality of partitions contains at least one of a first probe and a second probe, wherein the first probe binds to a target sequence that lacks the sequence variant and produces a first signal, and the second probe binds to a target sequence that contains the sequence variant and produces a second signal; d) detecting the first signal and the second signal from the individual partition; and e) identifying a false positive when a level of the first signal exceeds that of a threshold level indicative of one copy of the target sequence, and a level of the second signal exceeds that of a threshold level indicative of one copy of the target sequence.

In some cases, the methods may be suitable for use on samples with low starting amounts of polynucleotides. In such cases, the starting amount of polynucleotides may generally be too low for use in a digital PCR assay and may require one or more amplification steps prior to performing the digital PCR assay. However, such amplification steps may be prone to error, thereby increasing the number of false positives reported by a digital PCR assay. In some cases, the methods may reduce the number of false positives reported from a digital PCR assay. For example, the methods may reduce the number of false positives reported from a digital PCR assay by at least 1%, 2%, 3%, 4%, 5%, 6%, 7%, 8%, 9%, 10%, 15%, 20%, 25%, 30%, 35%, 40%, 45%, 50%, or greater than about 50%.

The starting amount of polynucleotides in a sample may be small. In some embodiments, the amount of starting polynucleotides is less than 50 ng, such as less than 45 ng, 40 ng, 35 ng, 30 ng, 25 ng, 20 ng, 15 ng, 10 ng, 5 ng, 4 ng, 3 ng, 2 ng, 1 ng, 0.5 ng, 0.1 ng, or less. In some embodiments, the amount of starting polynucleotides is in the range of 0.1-100 ng, such as between 1-75 ng, 5-50 ng, or 10-20 ng.

The polynucleotides may be from any suitable sample, such as a sample described herein with respect to the various aspects of the disclosure. Polynucleotides from a sample may be any of a variety of polynucleotides, including but not limited to, DNA, RNA, ribosomal RNA (rRNA), transfer RNA (tRNA), micro RNA (miRNA), messenger RNA (mRNA), cell-free DNA (cfDNA), circulating tumor DNA (ctDNA), fragments of any of these, or combinations of any two or more of these. In some embodiments, samples comprise DNA. In some embodiments, the polynucleotides are single-stranded, either as obtained or by way of treatment (e.g. denaturation). Further examples of suitable polynucleotides are described herein, such as with respect to any of the various aspects of the disclosure. In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, which may correspond to dead or damaged cells. The identity of such cells may be used to characterize the cells or population of cells from which they are derived, such as in a microbial community. If a sample is treated to extract polynucleotides, such as from cells in a sample, a variety of extraction methods are available, examples of which are provided herein (e.g. with regard to any of the various aspects of the disclosure).

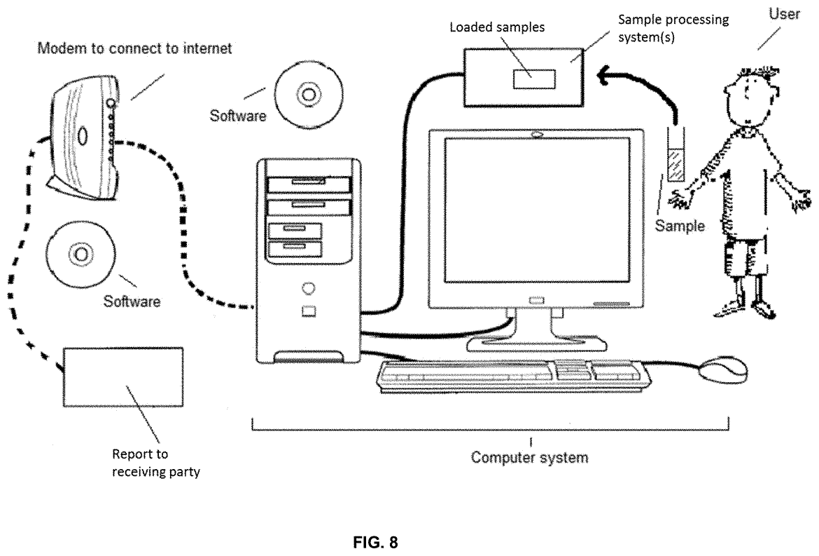

In one aspect, the disclosure provides a system for detecting a sequence variant. In some embodiments, a system may comprise: a) a computer configured to receive a user request to perform a detection reaction on a sample; b) an amplification system that performs a nucleic acid amplification reaction on the sample or a portion thereof in response to the user request, wherein the amplification reaction comprises: (i) circularizing individual polynucleotides of the sample to form a plurality of circularized polynucleotides; and (ii) amplifying the plurality of circularized polynucleotides to form a plurality of concatemers, each comprising a plurality of sequence repeats; c) a partitioning system that partitions the plurality of concatemers into a plurality of partitions, such that, on average, no more than one concatemer comprising a target sequence is present in an individual partition; and d) a detection system that detects a level of a first signal and a level of a second signal from an individual partition, wherein the first signal is generated when a first probe binds to a target sequence that lack the sequence variant, and the second signal is generated when a second probe binds to a target sequence that contains the sequence variant; and e) a report generator that sends a report to a recipient, wherein the report contains results for detection of the sequence variant. In some embodiments, the recipient is the user. FIG. 8 illustrates a non-limiting example of a system useful in the methods of the present disclosure.

A computer for use in the system can comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A client-server, relational database architecture can be used in embodiments of the system. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments, the server computer handles all of the database functionality. The client computer can have software that handles all the front-end data management and can also receive data input from users.

The system can be configured to receive a user request to perform a detection reaction on a sample. The user request may be direct or indirect. Examples of direct request include those transmitted by way of an input device, such as a keyboard, mouse, or touch screen). Examples of indirect requests include transmission via a communication medium, such as over the internet (either wired or wireless).

The system can further comprise an amplification system that performs a nucleic acid amplification reaction on the sample or a portion thereof in response to the user request. A variety of methods of amplifying polynucleotides (e.g., DNA and/or RNA) are available. Amplification may be linear, exponential, or involve both linear and exponential phases in a multi-phase amplification process. Amplification methods may involve changes in temperature, such as a heat denaturation step, or may be isothermal processes that do not require heat denaturation. Non-limiting examples of suitable amplification processes are described herein, such as with regard to any of the various aspects of the disclosure. In some embodiments, amplification comprises rolling circle amplification (RCA). A variety of systems for amplifying polynucleotides are available, and may vary based on the type of amplification reaction to be performed. For example, for amplification methods that comprise cycles of temperature changes, the amplification system may comprise a thermocycler. An amplification system can comprise a real-time amplification and detection instrument, such as systems manufactured by Applied Biosystems, Roche, and Strategene. In some embodiments, the amplification reaction comprises the steps of (i) circularizing individual polynucleotides of the sample to form a plurality of circularized polynucleotides; and (ii) amplifying the plurality of circularized polynucleotides to form a plurality of concatemers, each comprising a plurality of sequence repeats. Samples, polynucleotides, primers, polymerases, and other reagents can be any of those described herein, such as with regard to any of the various aspects. Non-limiting examples of circularization processes (e.g., with and without adapter oligonucleotides), reagents (e.g., types of adaptors, use of ligases), reaction conditions (e.g., favoring self-joining), and optional additional processing (e.g., post-reaction purification), are provided herein, such as with regard to any of the various aspects of the disclosure. Systems can be selected and or designed to execute any such methods.

Systems may further comprise a partitioning system that partitions the plurality of concatemers into a plurality of partitions, such that, on average, no more than one concatemer comprising a target sequence is present in an individual partition. Partitioning systems may include any number of systems that can separate a mixture comprising a plurality of polynucleotides into individual partitions. In some cases, the partitioning system is a droplet-based partitioning system, including microfluidic-based droplet systems, such as systems commercially available from Bio-Rad, Raindance Technologies, 10× Genomics, among others. In some cases, the partitioning system is a microplate-based partitioning system, such as systems commercially available from Becton, Dickinson and Company (Cellular Research), Mission Bio, Takara (WaferGen), among others.

The system may further comprise a detection system that detects a level of a first signal and a level of a second signal from an individual partition. In some cases, the first signal is generated when a first probe binds to a target sequence that lack the sequence variant, and the second signal is generated when a second probe binds to a target sequence that contains the sequence variant. The detection system may include any number of optical configurations, including, for example, a light source (e.g., a light-emitting diode (LED) for illuminating individual partitions, a lens, a filter, a dichroic mirror, or any combination thereof. The detection system may further include a photodetector for detecting an optical signal from the plurality of partitions.

The system can further comprise a report generator that sends a report to a recipient, wherein the report contains results for detection of the sequence variant. For example, the report generator may generate a report that identifies the presence of a sequence variant in the sample. Additionally or alternatively, the report may identify the absence of a sequence variant in the sample. Additionally or alternatively, the report may identify a false positive generated by the digital PCR assay. In some cases, the false positive may be excluded from the report. In other cases, the false positive may be flagged or identified on the report as a false positive. A report may be generated in real-time, with periodic updates as the process progresses. In addition, or alternatively, a report may be generated at the conclusion of the analysis. The report may be generated automatically, such as when the system completes the step of identifying the presence or absence of a sequence variant. In some embodiments, the report is generated in response to instructions from a user. In addition to the results of detection of the sequence variant, a report may also contain an analysis based on the one or more sequence variants. For example, where one or more sequence variants are associated with a particular contaminant or phenotype, the report may include information concerning this association, such as a likelihood that the contaminant or phenotype is present, at what level, and optionally a suggestion based on this information (e.g., additional tests, monitoring, or remedial measures). The report can take any of a variety of forms. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g., one or more computers, and/or one or more servers).

In another aspect, the disclosure provides a computer-readable medium comprising codes that, upon execution by one or more processors, implement a method of detecting a sequence variant. In some embodiments, the implemented method comprises: a) receiving a user request to perform a detection reaction on a sample; b) performing a nucleic acid amplification reaction on the sample or a portion thereof in response to the user request, wherein the amplification reaction comprises: (i) circularizing individual polynucleotides of the sample to form a plurality of circularized polynucleotides; and (ii) amplifying the plurality of circularized polynucleotides to form a plurality of concatemers, each comprising a plurality of sequence repeats; c) partitioning the plurality of concatemers into a plurality of partitions, such that, on average, no more than one concatemer comprising a target sequence is present in an individual partition, wherein an individual partition of the plurality of partitions contains at least one of a first probe and a second probe, wherein the first probe binds to the plurality of sequence repeats that lack the sequence variant and produces a first signal, and the second probe binds to the plurality of sequence repeats that contain the sequence variant and produces a second signal; d) detecting the first signal and the second signal from the individual partition; and e) identifying the sequence variant as present only when a level of the second signal exceeds that of a threshold level indicative of one copy of a target sequence, and a level of the first signal is below a threshold level indicative of one copy of a target sequence; and f) generating a report that contains results for detection of the sequence variant.

In some embodiments, the implemented method further comprises identifying the sequence variant as absent when a level of the first signal exceeds that of a threshold level indicative of one copy of a target sequence, and a level of the second signal is below a threshold level indicative of one copy of a target sequence. In some embodiments, the implemented method further comprises identifying a false positive when a level of the first signal exceeds that of a threshold level indicative of one copy of a target sequence, and a level of the second signal exceeds that of a threshold level of one copy of a target sequence.

A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computers) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device comprising a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch -sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

In some embodiments of any of the various aspects disclosed herein, the methods, compositions, and systems have therapeutic applications, such as in the characterization of a patient sample and optionally diagnosis of a condition of a subject. Therapeutic applications may also include informing the selection of therapies to which a patient may be most responsive (also referred to as “theranostics”), and actual treatment of a subject in need thereof, based on the results of a method described herein. In particular, methods and compositions disclosed herein may be used to diagnose tumor presence, progression and/or metastasis of tumors, especially when the polynucleotides analyzed comprise or consist of cfDNA, ctDNA, or fragmented tumor DNA. In some embodiments, a subject is monitored for treatment efficacy. For example, by monitoring ctDNA over time, a decrease in ctDNA can be used as an indication of efficacious treatment, while increases can facilitate selection of different treatments or different dosages. Other uses include evaluations of organ rejection in transplant recipients (where increases in the amount of circulating DNA corresponding to the transplant donor genome is used as an early indicator of transplant rejection), and genotyping/isotyping of pathogen infections, such as viral or bacterial infections. Detection of sequence variants in circulating fetal DNA may be used to diagnose a condition of a fetus.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. Typically, prophylactic benefit includes reducing the incidence and/or worsening of one or more diseases, conditions, or symptoms under treatment (e.g. as between treated and untreated populations, or between treated and untreated states of a subject). Improving a treatment outcome may include diagnosing a condition of a subject in order to identify the subject as one that will or will not benefit from treatment with one or more therapeutic agents, or other therapeutic intervention (such as surgery). In such diagnostic applications, the overall rate of successful treatment with the one or more therapeutic agents may be improved, relative to its effectiveness among patients grouped without diagnosis according to a method of the present disclosure (e.g. an improvement in a measure of therapeutic efficacy by at least about 10%, 20%, 30%, 40%, 50%, 60%, 70%, 80%, 90%, 95%, or more).

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In some embodiments of the various methods described herein, the sample is from a subject. A subject can be any organism, non-limiting examples of which include plants, animals, fungi, protists, monerans, viruses, mitochondria, and chloroplasts. Sample polynucleotides can be isolated from a subject, such as a cell sample, tissue sample, bodily fluid sample, or organ sample (or cell cultures derived from any of these), including, for example, cultured cell lines, biopsy, blood sample, cheek swab, or fluid sample containing a cell (e.g. saliva). In some cases, the sample does not comprise intact cells, is treated to remove cells, or polynucleotides are isolated without a cellular extractions step (e.g. to isolate cell-free polynucleotides, such as cell-free DNA). Other examples of sample sources include those from blood, urine, feces, nares, the lungs, the gut, other bodily fluids or excretions, materials derived therefrom, or combinations thereof. The subject may be an animal, including but not limited to, a cow, a pig, a mouse, a rat, a chicken, a cat, a dog, etc., and is usually a mammal, such as a human. In some embodiments, the sample comprises tumor cells, such as in a sample of tumor tissue from a subject. In some embodiments, the sample is a blood sample or a portion thereof (e.g. blood plasma or serum). Serum and plasma may be of particular interest, due to the relative enrichment for tumor DNA associated with the higher rate of malignant cell death among such tissues. A sample may be a fresh sample, or a sample subjected to one or more storage processes (e.g. paraffin-embedded samples, particularly formalin-fixed paraffin-embedded (FFPE) sample). In some embodiments, a sample from a single individual is divided into multiple separate samples (e.g. 2, 3, 4, 5, 6, 7, 8, 9, 10, or more separate samples) that are subjected to methods of the disclosure independently, such as analysis in duplicate, triplicate, quadruplicate, or more. Where a sample is from a subject, the reference sequence may also be derived from the subject, such as a consensus sequence from the sample under analysis or the sequence of polynucleotides from another sample or tissue of the same subject. For example, a blood sample may be analyzed for ctDNA mutations, while cellular DNA from another sample (e.g. buccal or skin sample) is analyzed to determine the reference sequence.

Polynucleotides may be extracted from a sample, with or without extraction from cells in a sample, according to any suitable method. A variety of kits are available for extraction of polynucleotides, selection of which may depend on the type of sample, or the type of nucleic acid to be isolated. Examples of extraction methods are provided herein, such as those described with respect to any of the various aspects disclosed herein. In one example, the sample may be a blood sample, such as a sample collected in an EDTA tube (e.g., BD Vacutainer). Plasma can be separated from the peripheral blood cells by centrifugation (e.g. 10 minutes at 1900 ×g at 4° C.). Plasma separation performed in this way on a 6mL blood sample will typically yield 2.5 to 3 mL of plasma. Circulating cell-free DNA can be extracted from a plasma sample, such as by using a QIAmp Circulating Nucleic Acid Kit (Qiagene), according the manufacturer's protocol. DNA may then be quantified (e.g. on an Agilent 2100 Bioanalyzer with High Sensitivity DNA kit (Agilent)). As an example, yield of circulating DNA from such a plasma sample from a healthy person may range from 1 ng to 10 ng per mL of plasma, with significantly more in cancer patient samples.

Polynucleotides can also be derived from stored samples, such frozen or archived samples. One common method for storing samples is to formalin-fix and paraffin-embed them. However, this process is also associated with degradation of nucleic acids. Polynucleotides processed and analyzed from an FFPE sample may include short polynucleotides, such as fragments in the range of 50-200 base pairs, or shorter. A number of techniques exist for the purification of nucleic acids from fixed paraffin-embedded samples, such as those described in WO2007133703, and methods described by Foss, et al Diagnostic Molecular Pathology, (1994) 3:148-155 and Paska, C., et al Diagnostic Molecular Pathology, (2004) 13:234-240. Commercially available kits may be used for purifying polynucleotides from FFPE samples, such as Ambion's Recoverall Total Nucleic acid Isolation kit. Typical methods start with a step that removes the paraffin from the tissue via extraction with Xylene or other organic solvent, followed by treatment with heat and a protease like proteinase K which cleaves the tissue and proteins and helps to release the genomic material from the tissue. The released nucleic acids can then be captured on a membrane or precipitated from solution, washed to removed impurities and for the case of mRNA isolation, a DNase treatment step is sometimes added to degrade unwanted DNA. Other methods for extracting FFPE DNA are available and can be used in the methods of the present disclosure.

In some embodiments, the plurality of polynucleotides comprise cell-free polynucleotides, such as cell-free DNA (cfDNA) or circulating tumor DNA (ctDNA). Cell-free DNA circulates in both healthy and diseased individuals. cfDNA from tumors (ctDNA) is not confined to any specific cancer type, but appears to be a common finding across different malignancies. According to some measurements, the free circulating DNA concentration in plasma is about 14-18 ng/ml in control subjects and about 180-318 ng/ml in patients with neoplasias. Apoptotic and necrotic cell death contribute to cell-free circulating DNA in bodily fluids. For example, significantly increased circulating DNA levels have been observed in plasma of prostate cancer patients and other prostate diseases, such as Benign Prostate Hyperplasia and Prostatitis. In addition, circulating tumor DNA is present in fluids originating from the organs where the primary tumor occurs. Thus, breast cancer detection can be achieved in ductal lavages; colorectal cancer detection in stool; lung cancer detection in sputum, and prostate cancer detection in urine or ejaculate. Cell-free DNA may be obtained from a variety of sources. One common source is blood samples of a subject. However, cfDNA or other fragmented DNA may be derived from a variety of other sources. For example, urine and stool samples can be a source of cfDNA, including ctDNA.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides. For example, cell-free polynucleotides may include cell-free DNA (also called “circulating” DNA). In some embodiments, the circulating DNA is circulating tumor DNA (ctDNA) from tumor cells, such as from a body fluid or excretion (e.g., blood sample). Tumors frequently show apoptosis or necrosis, such that tumor nucleic acids are released into the body, including the blood stream of a subject, through a variety of mechanisms, in different forms and at different levels. Typically, the size of the ctDNA can range between higher concentrations of smaller fragments, generally 70 to 200 nucleotides in length, to lower concentrations of large fragments of up to thousands kilobases.

In some embodiments of any of the various aspects described herein, detecting a sequence variant comprises detecting mutations (e.g., rare somatic mutations) with respect to a reference sequence or in a background of no mutations, where the sequence variant is correlated with disease. In general, sequence variants for which there is statistical, biological, and/or functional evidence of association with a disease or trait are referred to as “causal genetic variants.” A single causal genetic variant can be associated with more than one disease or trait. In some embodiments, a causal genetic variant can be associated with a Mendelian trait, a non-Mendelian trait, or both. Causal genetic variants can manifest as variations in a polynucleotide, such 1, 2, 3, 4, 5, 6, 7, 8, 9, 10, 20, 50, or more sequence differences (such as between a polynucleotide comprising the causal genetic variant and a polynucleotide lacking the causal genetic variant at the same relative genomic position). Non-limiting examples of types of causal genetic variants include single nucleotide polymorphisms (SNP), deletion/insertion polymorphisms (DIP), copy number variants (CNV), short tandem repeats (STR), restriction fragment length polymorphisms (RFLP), simple sequence repeats (SSR), variable number of tandem repeats (VNTR), randomly amplified polymorphic DNA (RAPD), amplified fragment length polymorphisms (AFLP), inter-retrotransposon amplified polymorphisms (TRAP), long and short interspersed elements (LINE/SINE), long tandem repeats (LTR), mobile elements, retrotransposon microsatellite amplified polymorphisms, retrotransposon-based insertion polymorphisms, sequence specific amplified polymorphism, and heritable epigenetic modification (for example, DNA methylation). A causal genetic variant may also be a set of closely related causal genetic variants. Some causal genetic variants may exert influence as sequence variations in RNA polynucleotides. At this level, some causal genetic variants are also indicated by the presence or absence of a species of RNA polynucleotides. Also, some causal genetic variants result in sequence variations in protein polypeptides. A number of causal genetic variants have been reported. An example of a causal genetic variant that is a SNP is the Hb S variant of hemoglobin that causes sickle cell anemia. An example of a causal genetic variant that is a DIP is the delta508 mutation of the CFTR gene which causes cystic fibrosis. An example of a causal genetic variant that is a CNV is trisomy 21, which causes Down's syndrome. An example of a causal genetic variant that is an STR is tandem repeat that causes Huntington's disease. Non-limiting examples of causal genetic variants and diseases with which they are associated are provided in Table 1. Additional non-limiting examples of causal genetic variants are described in WO2014015084. Further examples of genes in which mutations are associated with diseases, and in which sequence variants may be detected according to a method of the disclosure, are provided in Table 2.

TABLE 1
Causal genetic variants and diseases with which they are associated

| Disease | Gene | Variant Name |
|---|---|---|
| 21-Hydroxylase | CYP21A2 | F306 + t |
| Deficiency | | |
| 21-Hydroxylase | CYP21A2 | F306 + t |
| Deficiency | | |
| 21-Hydroxylase | CYP21A3 | g.655A/C > G |
| Deficiency | | |
| 21-Hydroxylase | CYP21A4 | g.655A/C > G |
| Deficiency | | |
| 21-Hydroxylase | CYP21A6 | G110del8nt |
| Deficiency | | |
| 21-Hydroxylase | CYP21A5 | G110del8nt |
| Deficiency | | |
| 21-Hydroxylase | CYP21A7 | I172N, rs34607927 |
| Deficiency | | |
| 21-Hydroxylase | CYP21A2 | I236N |
| Deficiency | | |
| 21-Hydroxylase | CYP21A2 | M239K, rs6476 |
| Deficiency | | |
| 21-Hydroxylase | | |
| Deficiency | CYP21A2 | P30L |
| 21-Hydroxylase | | |
| Deficiency | CYP21A2 | P453S |
| 21-Hydroxylase | | |
| Deficiency | CYP21A2 | Q318X |
| 21-Hydroxylase | | |
| Deficiency | CYP21A2 | R356W |
| 21-Hydroxylase | | |
| Deficiency | CYP21A2 | V237E, rs12530380 |
| 21-Hydroxylase | CYP21A2 | V281L, rs6471 |
| Deficiency | | |
| ABCC8-Related | ABCC8 | 3992-9G > A |
| Hyperinsulinism | | |
| ABCC8-Related | ABCC8 | delF1388 |
| Hyperinsulinism | | |
| ABCC8-Related | ABCC8 | delF1388 |
| Hyperinsulinism | | |
| ABCC8-Related | ABCC8 | V187D |
| Hyperinsulinism | | |
| Achondroplasia | FGFR3 | G375C |
| Achondroplasia | FGFR3 | G380R, rs28931614 |
| Achromatopsia | CNGB3 | c.1148delC |
| Achromatopsia | CNGB3 | c.1148delC |
| Achromatopsia | CNGB3 | c.819-826del8 |
| Achromatopsia | CNGB3 | c.819-826del8 |
| Achromatopsia | CNGB3 | c.886-896del11insT |
| Achromatopsia | CNGB3 | c.886-896del11insT |
| Achromatopsia | CNGB3 | c.991 − 3T > G |
| Achromatopsia | CNGB3 | p.Arg403Gln |
| Achromatopsia | CNGB3 | p.Glu336X |
| Adenosine | | |
| Monophosphate | AMPD1 | P48L |
| Deaminase 1 | | |
| Adenosine | | |
| Monophosphate | AMPD1 | Q12X, rs17602729 |
| Deaminase 1 | | |
| Agenesis of Corpus | SLC12A6 | c.2436delG |
| Callosum with | | |
| Neuronopathy | | |
| Agenesis of Corpus | SLC12A6 | c.2436delG |
| Callosum with | | |
| Neuronopathy | | |
| Alkaptonuria | HGD | c.174delA |
| Alkaptonuria | HGD | c.174delA |
| Alkaptonuria | HGD | c.457_458insG |
| Alkaptonuria | HGD | c.457_458insG |
| Alkaptonuria | HGD | G161R |
| Alkaptonuria | HGD | G270R |
| Alkaptonuria | HGD | IVS1 − 1G > A |
| Alkaptonuria | HGD | IVS5 + 1G > A |
| Alkaptonuria | HGD | Met368Val |
| Alkaptonuria | HGD | P230S |
| Alkaptonuria | HGD | S47L |
| Alkaptonuria | HGD | V300G |
| Alpha-1-Antitrypsin | SERPINA1 | Arg101His, rs709932 |
| Deficiency | | |
| Alpha-1-Antitrypsin | SERPINA1 | Glu264Val |
| Deficiency | | |
| Alpha-1-Antitrypsin | SERPINA1 | Glu342Lys, rs28929474 |
| Deficiency | | |
| Alpha-1-Antitrypsin | SERPINA1 | Glu376Asp, rs1303 |
| Deficiency | | |
| Alpha-Mannosidosis | MAN2B1 | IVS14 + 1G > C |
| Alpha-Mannosidosis | MAN2B1 | p.L809P |
| Alpha-Mannosidosis | MAN2B1 | p.R750W |
| Alpha- | SGCA | R77C, rs28933693 |
| Sarcoglycanopathy | | |
| Alpha-Thalassemia | HBA2 | H19D |
| Alpha-Thalassemia | HBA1 | HbQ |
| Alpha-Thalassemia | HBA1, HBA2 | 3.7 kb (type I) deletion alpha-2 |
| Alpha-Thalassemia | HBA1, HBA2 | 3.7 kb (type I) deletion alpha-2 |
| Alpha-Thalassemia | HBA1, HBA2 | HbVar database id #1086, --(SEA); deletion of ~20 kb |
| | | including both alpha-globin genes alpha-Thal-1 |
| Alpha-Thalassemia | HBA1, HBA2 | HbVar database id #1086, --(SEA); deletion of ~20 kb |
| | | including both alpha-globin genes alpha-Thal-1 |
| Alpha-Thalassemia | HBA1, HBA2 | HbVar database id #1087, --(MED-I); deletion of ~17.5 |
| | | kb including both alpha-globin genes alpha-Thal-1 |
| Alpha-Thalassemia | HBA1, HBA2 | HbVar database id #1087, --(MED-I); deletion of ~17.5 |
| | | kb including both alpha-globin genes alpha-Thal-1 |
| Alpha-Thalassemia | HBA1, HBA2 | HbVar database id #1088, -(alpha)20.5; this 20.5 kb |
| | | deletion involves alpha2 and the 5′ end of alpha1; alpha- |
| | | Thal-1 |
| Alpha-Thalassemia | HBA1, HBA2 | HbVar database id #1088, -(alpha)20.5; this 20.5 kb |
| | | deletion involves alpha2 and the 5′ end of alpha1; alpha- |
| | | Thal-1 |
| Alpha-Thalassemia | HBA1, HBA2 | HbVar database id #1094, --(FIL); a deletion of 30-34 |
| | | kb involving the alpha1, alpha2, and zeta genes alpha- |
| | | Thal-1 |
| Alpha-Thalassemia | HBA1, HBA2 | HbVar database id #1094, --(FIL); a deletion of 30-34 |
| | | kb involving the alpha1, alpha2, and zeta genes alpha- |
| | | Thal-1 |
| Alpha-Thalassemia | HBA1, HBA2 | HbVar database id #1095, --(THAI); a deletion of 34-38 |
| | | kb involving the alpha1, alpha2, and zeta genes alpha- |
| | | Thal-1 |
| Alpha-Thalassemia | HBA1, HBA2 | HbVar database id #1095, --(THAI); a deletion of 34-38 |
| | | kb involving the alpha1, alpha2, and zeta genes alpha- |
| | | Thal-1 |
| Alpha-Thalassemia | HBA1, HBA2 | HbVar database id #1097, --(MED-II); a deletion of 26.5 |
| | | kb involving the two alpha and zeta genes alpha-Thal-1 |
| Alpha-Thalassemia | HBA1, HBA2 | HbVar database id #1097, --(MED-II); a deletion of 26.5 |
| | | kb involving the two alpha and zeta genes alpha-Thal-1 |
| Alpha-Thalassemia | HBA2 | HbVar database id #187 |
| Alpha-Thalassemia | HBA2 | HbVar database id #2598, IVS I-5 (G > A) |
| Alpha-Thalassemia | HBA1, HBA2 | HbVar database id #703 |
| Alpha-Thalassemia | HBA1, HBA2 | HbVar database id #704 |
| Alpha-Thalassemia | HBA1, HBA2 | HbVar database id #705, Hb Koya Dora |
| Alpha-Thalassemia | HBA1, HBA2 | HbVar database id #707, rs41412046 |
| Alpha-Thalassemia | HBA1 | HbVar database id #87 |
| Alpha-Thalassemia | HBA1, HBA2 | HbVar database id #969, Poly A (A → G); AATAAA- |
| | | > AATGAA beta+ |
| Alpha-Thalassemia | HBA1, HBA2 | HbVar database id #971, Poly A (A → G); AATAAA- |
| | | > AATAAG beta+ |
| Alpha-Thalassemia | HBA2 | M1T |
| Alpha-Thalassemia | HBA1 | W14X |
| Angiotensin II | AGTR1 | A1166C |
| Receptor, Type 1 | | |
| Apolipoprotein E | APOE | p.C112R, rs429358 |
| Genotyping | | |
| Apolipoprotein E | APOE | p.R158C, rs7412 |
| Genotyping | | |
| Argininosuccinicaciduria | ASL | R385C |
| ARSACS | SACS | 5254C > T |
| ARSACS | SACS | 6594delT |
| ARSACS | SACS | 6594delT |
| Aspartylglycosaminuria | AGA | c.199_200delGA |
| Aspartylglycosaminuria | AGA | c.199_200delGA |
| Aspartylglycosaminuria | AGA | C163S |
| Ataxia with Vitamin E | TTPA | 744delA |
| Deficiency | | |
| Ataxia with Vitamin E | TTPA | 744delA |
| Deficiency | | |
| Ataxia-Telangiectasia | ATM | R35X |
| Autoimmune | AIRE | c.1163_1164insA |
| Polyendocrinopathy | | |
| Syndrome Type 1 | | |
| Autoimmune | AIRE | c.1163_1164insA |
| Polyendocrinopathy | | |
| Syndrome Type 1 | | |
| Autoimmune | AIRE | c.769C > T |
| Polyendocrinopathy | | |
| Syndrome Type 1 | | |
| Autoimmune | AIRE | c.967_979del |
| Polyendocrinopathy | | |
| Syndrome Type 1 | | |
| Autoimmune | AIRE | c.967_979del |
| Polyendocrinopathy | | |
| Syndrome Type 1 | | |
| Autoimmune | AIRE | Y85C |
| Polyendocrinopathy | | |
| Syndrome Type 1 | | |
| Bardet-Biedl Syndrome | BBS1 | M390R |
| Bardet-Biedl Syndrome | BBS10 | p.C91LfsX4 |
| Bardet-Biedl Syndrome | BBS10 | p.C91LfsX4 |
| Best Vitelliform | BEST1 | c.G383C |
| Macular Dystrophy | | |
| Beta- | SGCB | S114F |
| Sarcoglycanopathy | | |
| Beta-Thalassemia | HBB | −28 (A → G) beta+ |
| Beta-Thalassemia | HBB | −29 (A → G) beta+ |
| Beta-Thalassemia | HBB | −29 A > G |
| Beta-Thalassemia | HBB | −30 (T → A) beta+ |
| Beta-Thalassemia | HBB | −87 (C → G) beta+ |
| Beta-Thalassemia | HBB | −88C > T |
| Beta-Thalassemia | HBB | CAP + 1 (A → C) beta+ |
| Beta-Thalassemia | HBB | Codon 15 (G → A); TGG(Trp) → TAG(stop codon) beta0, |
| | | rs34716011 |
| Beta-Thalassemia | HBB | Codon 15 (G → A); TGG(Trp) → TAG(stop codon) beta0, |
| | | rs34716011 |
| Beta-Thalassemia | HBB | Codon 16 (−C); GGC(Gly) → GG-beta0 |
| Beta-Thalassemia | HBB | Codon 16 (−C); GGC(Gly) → GG-beta0 |
| Beta-Thalassemia | HBB | Codon 17 (A → T); AAG(Lys) → TAG(stop codon) beta0 |
| Beta-Thalassemia | HBB | Codon 24 (T → A); GGT(Gly) → GGA(Gly) beta+ |
| Beta-Thalassemia | HBB | Codon 39 (C → T); CAG(Gln) → TAG(stop codon) beta0 |
| Beta-Thalassemia | HBB | Codon 5 (−CT); CCT(Pro) → C--beta0 |
| Beta-Thalassemia | HBB | Codon 5 (−CT); CCT(Pro) → C--beta0 |
| Beta-Thalassemia | HBB | Codon 6 (−A); GAG(Glu) → G-G beta0 |
| Beta-Thalassemia | HBB | Codon 6 (−A); GAG(Glu) → G-G beta0 |
| Beta-Thalassemia | HBB | Codon 8 (−AA); AAG(Lys) → --G beta0 |
| Beta-Thalassemia | HBB | Codon 8 (−AA); AAG(Lys) → --G beta0 |
| Beta-Thalassemia | HBB | Codons 41/42 (−TTCT); TTCTTT(Phe-Phe) → ----TT |
| | | beta0 |
| Beta-Thalassemia | HBB | Codons 41/42 (−TTCT); TTCTTT(Phe-Phe) → ----TT |
| | | beta0 |
| Beta-Thalassemia | HBB | Codons 71/72 (+A); TTT AGT(Phe Ser) → TTT A AGT; |
| | | beta0 |
| Beta-Thalassemia | HBB | Codons 71/72 (+A); TTT AGT(Phe Ser) → TTT A AGT; |
| | | beta0 |
| Beta-Thalassemia | HBB | Codons 8/9 (+G); AAG TCT(Lys;Ser) → AAG G TCT |
| | | beta0 |
| Beta-Thalassemia | HBB | Codons 8/9 (+G); AAG TCT(Lys;Ser) → AAG G TCT |
| | | beta0 |
| Beta-Thalassemia | HBB | HbVar database id #889, IVS-II-654 (C → T); |
| | | AAGGCAATA → AAG\hat{ }AGTAATA beta+ (severe) |
| Beta-Thalassemia | HBB | HbVar database id #890, IVS-II-705 (T → G); |
| | | GATGTAAGA → GAG\hat{ }AGTAAGA beta+ |
| Beta-Thalassemia | HBB | HbVar database id #891, IVS-II-745 (C → G); |
| | | CAGCTACCAT → CAG\hat{ }AGTACCAT beta+ |
| Beta-Thalassemia | HBB | HbVar database id #979, 619 bp deletion beta0 |
| Beta-Thalassemia | HBB | 619 bp deletion beta0 |
| Beta-Thalassemia | HBB | IVS-I-1 (G → A); AG\hat{ }AGTTGGT → AGATTGGT beta0 |
| Beta-Thalassemia | HBB | IVS-I-1 (G → T); AG\hat{ }AGTTGGT → AGTTTGGT beta0 |
| Beta-Thalassemia | HBB | IVS-I-110 (G → A) beta+ ; the mutation is 21 nucleotides |
| | | 5′ to the acceptor splice site AG\hat{ }AGC |
| Beta-Thalassemia | HBB | IVS-I-5 (G → C) beta+ (severe) |
| Beta-Thalassemia | HBB | IVS-II-1 (G → A); beta 0 |
| Beta-Thalassemia | HBB | IVS-II-844 (C → G); beta+ |
| Beta-Thalassemia | HBB | IVS1 + 6T > C |
| Beta-Thalassemia | HBB | IVS11-849A > C |
| Beta-Thalassemia | HBB | IVS11-849A > G |
| Biotinidase Deficiency | BTD | A171T, rs13073139 |
| Biotinidase Deficiency | BTD | D252G, rs28934601 |
| Biotinidase Deficiency | BTD | D444H, rs13078881 |
| Biotinidase Deficiency | BTD | F403V |
| Biotinidase Deficiency | BTD | G98:d7i3 |
| Biotinidase Deficiency | BTD | G98:d7i3 |
| Biotinidase Deficiency | BTD | Q456H |
| Biotinidase Deficiency | BTD | R157H |
| Biotinidase Deficiency | BTD | R538C |
| Blau Syndrome | NOD2 | E383K |
| Blau Syndrome | NOD2 | L469F |
| Blau Syndrome | NOD2 | R334Q |
| Blau Syndrome | NOD2 | R334W |
| Bloom Syndrome | BLM | 2407insT |
| Bloom Syndrome | BLM | 2407insT |
| Bloom Syndrome | BLM | 736delATCTGAinsTAGATTC (2281del6/ins7) |
| Bloom Syndrome | BLM | 736delATCTGAinsTAGATTC (2281del6/ins7) |
| BRCA1 Hereditary | BRCA1 | 185delAG |
| Breast/Ovarian Cancer | | |
| BRCA1 Hereditary | BRCA1 | 185delAG |
| Breast/Ovarian Cancer | | |
| BRCA1 Hereditary | BRCA1 | 5382insC |
| Breast/Ovarian Cancer | | |
| BRCA1 Hereditary | BRCA1 | 5382insC |
| Breast/Ovarian Cancer | | |
| BRCA1 Hereditary | BRCA1 | Tyr978X |
| Breast/Ovarian Cancer | | |
| BRCA2 Hereditary | BRCA2 | 6174delT |
| Breast/Ovarian Cancer | | |
| BRCA2 Hereditary | BRCA2 | 6174delT |
| Breast/Ovarian Cancer | | |
| BRCA2 Hereditary | BRCA2 | 8765delAG |
| Breast/Ovarian Cancer | | |
| BRCA2 Hereditary | BRCA2 | 8765delAG |
| Breast/Ovarian Cancer | | |
| Canavan Disease | ASPA | A305E (914C > A), rs28940574 |
| Canavan Disease | ASPA | E285A (854A > C), rs28940279 |
| Canavan Disease | ASPA | IVS2-2A > G (433-2A > G) |
| Canavan Disease | ASPA | Y231X (693C > A) |
| Carnitine | CPT1A | G710E |
| Palmitoyltransferase IA | | |
| Deficiency | | |
| Carnitine | CPT1A | P479L |
| Palmitoyltransferase IA | | |
| Deficiency | | |
| Carnitine | CPT2 | G549D |
| Palmitoyltransferase II | | |
| Deficiency | | |
| Carnitine | CPT2 | L178F 534 ins/25 bp del |
| Palmitoyltransferase II | | |
| Deficiency | | |
| Carnitine | CPT2 | L178F 534 ins/25 bp del |
| Palmitoyltransferase II | | |
| Deficiency | | |
| Carnitine | CPT2 | P227L |
| Palmitoyltransferase II | | |
| Deficiency | | |
| Carnitine | CPT2 | P50H |
| Palmitoyltransferase II | | |
| Deficiency | | |
| Carnitine | CPT2 | P604S |
| Palmitoyltransferase II | | |
| Deficiency | | |
| Carnitine | CPT2 | Q413fs |
| Palmitoyltransferase II | | |
| Deficiency | | |
| Carnitine | CPT2 | Q413fs |
| Palmitoyltransferase II | | |
| Deficiency | | |
| Carnitine | CPT2 | Q550R |
| Palmitoyltransferase II | | |
| Deficiency | | |
| Carnitine | CPT2 | R124X |
| Palmitoyltransferase II | | |
| Deficiency | | |
| Carnitine | CPT2 | R503C |
| Palmitoyltransferase II | | |
| Deficiency | | |
| Carnitine | CPT2 | R631C |
| Palmitoyltransferase II | | |
| Deficiency | | |
| Carnitine | CPT2 | S113L |
| Palmitoyltransferase II | | |
| Deficiency | | |
| Carnitine | CPT2 | s38fs |
| Palmitoyltransferase II | | |
| Deficiency | | |
| Carnitine | CPT2 | s38fs |
| Palmitoyltransferase II | | |
| Deficiency | | |
| Carnitine | CPT2 | Y6285, rs28936673 |
| Palmitoyltransferase II | | |
| Deficiency | | |
| Cartilage-Hair | RMRP | g.262G > T |
| Hypoplasia | | |
| Cartilage-Hair | RMPR | g.70A > G |
| Hypoplasia | | |
| CFTR-Related | CFTR | 1811 + 1.6 kb A → G |
| Disorders | | |
| CFTR-Related | CFTR | 2183AA > G |
| Disorders | | |
| CFTR-Related | CFTR | 2183AA > G |
| Disorders | | |
| CFTR-Related | CFTR | 3849 + 10 kb C > T |
| Disorders | | |
| CFTR-Related | CFTR | A455E |
| Disorders | | |
| CFTR-Related | CFTR | A559T |
| Disorders | | |
| CFTR-Related | CFTR | C524X |
| Disorders | | |
| CFTR-Related | CFTR | 574delA, 574delA |
| Disorders | | |
| CFTR-Related | CFTR | 574delA, 574delA |
| Disorders | | |
| CFTR-Related | CFTR | 2108delA, 2108delA |
| Disorders | | |
| CFTR-Related | CFTR | 2108delA, 2108delA |
| Disorders | | |
| CFTR-Related | CFTR | 3171delC, 3171delC |
| Disorders | | |
| CFTR-Related | CFTR | 3171delC, 3171delC |
| Disorders | | |
| CFTR-Related | CFTR | 621 + 1G → T |
| Disorders | | |
| CFTR-Related | CFTR | 2105-2117del13insAGAAA |
| Disorders | | |
| CFTR-Related | CFTR | 2105-2117del13insAGAAA |
| Disorders | | |
| CFTR-Related | CFTR | 711 + 1G → T |
| Disorders | | |
| CFTR-Related | CFTR | 711 + 5G → A |
| Disorders | | |
| CFTR-Related | CFTR | 712-1G → T |
| Disorders | | |
| CFTR-Related | CFTR | 1288insTA, 1288insTA |
| Disorders | | |
| CFTR-Related | CFTR | 1288insTA, 1288insTA |
| Disorders | | |
| CFTR-Related | CFTR | 936delTA |
| Disorders | | |
| CFTR-Related | CFTR | 936delTA |
| Disorders | | |
| CFTR-Related | CFTR | [delta]F311 |
| Disorders | | |
| CFTR-Related | CFTR | [delta]F311 |
| Disorders | | |
| CFTR-Related | CFTR | 1078delT, 1078delT |
| Disorders | | |
| CFTR-Related | CFTR | 1078delT, 1078delT |
| Disorders | | |
| CFTR-Related | CFTR | 1161delC, 1161delC |
| Disorders | | |
| CFTR-Related | CFTR | 1161delC, 1161delC |
| Disorders | | |
| CFTR-Related | CFTR | 1609delCA, 1609delCA |
| Disorders | | |
| CFTR-Related | CFTR | 1609delCA, 1609delCA |
| Disorders | | |
| CFTR-Related | CFTR | [delta]I507 |
| Disorders | | |
| CFTR-Related | CFTR | [delta]I507 |
| Disorders | | |
| CFTR-Related | CFTR | rs332, [delta]F508 |
| Disorders | | |
| CFTR-Related | CFTR | rs332, [delta]F508 |
| Disorders | | |
| CFTR-Related | CFTR | 1677delTA, 1677delTA |
| Disorders | | |
| CFTR-Related | CFTR | 1677delTA, 1677delTA |
| Disorders | | |
| CFTR-Related | CFTR | 1717-1G → A |
| Disorders | | |
| CFTR-Related | CFTR | 1812-1G → A |
| Disorders | | |
| CFTR-Related | CFTR | 1898 + 1G → A |
| Disorders | | |
| CFTR-Related | CFTR | 1898 + 1G → T |
| Disorders | | |
| CFTR-Related | CFTR | 1898 + 5G → T |
| Disorders | | |
| CFTR-Related | CFTR | 1949del84, 1949del84 |
| Disorders | | |
| CFTR-Related | CFTR | 1949del84, 1949del84 |
| Disorders | | |
| CFTR-Related | CFTR | 2043delG, 2043delG |
| Disorders | | |
| CFTR-Related | CFTR | 2043delG, 2043delG |
| Disorders | | |
| CFTR-Related | CFTR | 2055del9 → A |
| Disorders | | |
| CFTR-Related | CFTR | 2055del9 → A |
| Disorders | | |
| CFTR-Related | CFTR | 2143delT, 2143delT |
| Disorders | | |
| CFTR-Related | CFTR | 2143delT, 2143delT |
| Disorders | | |
| CFTR-Related | CFTR | 2184delA, 2184delA |
| Disorders | | |
| CFTR-Related | CFTR | 2184delA, 2184delA |
| Disorders | | |
| CFTR-Related | CFTR | 2184insA, 2184insA |
| Disorders | | |
| CFTR-Related | CFTR | 2184insA, 2184insA |
| Disorders | | |
| CFTR-Related | CFTR | 2307insA, 2307insA |
| Disorders | | |
| CFTR-Related | CFTR | 2307insA, 2307insA |
| Disorders | | |
| CFTR-Related | CFTR | 296 + 12T → C |
| Disorders | | |
| CFTR-Related | CFTR | 2789 + 5G → A |
| Disorders | | |
| CFTR-Related | CFTR | 2869insG, 2869insG |
| Disorders | | |
| CFTR-Related | CFTR | 2869insG, 2869insG |
| Disorders | | |
| CFTR-Related | CFTR | 3120G → A |
| Disorders | | |
| CFTR-Related | CFTR | 3120 + 1G → A |
| Disorders | | |
| CFTR-Related | CFTR | 3272-26A → G |
| Disorders | | |
| CFTR-Related | CFTR | 3659delC, 3659delC |
| Disorders | | |
| CFTR-Related | CFTR | 3659delC, 3659delC |
| Disorders | | |
| CFTR-Related | CFTR | 3667del4, 3667del4 |
| Disorders | | |
| CFTR-Related | CFTR | 3667del4, 3667del4 |
| Disorders | | |
| CFTR-Related | CFTR | 3791delC, 3791delC |
| Disorders | | |
| CFTR-Related | CFTR | 3791delC, 3791delC |
| Disorders | | |
| CFTR-Related | CFTR | 3821delT, 3821delT |
| Disorders | | |
| CFTR-Related | CFTR | 3821delT, 3821delT |
| Disorders | | |
| CFTR-Related | CFTR | 3905insT, 3905insT |
| Disorders | | |
| CFTR-Related | CFTR | 3905insT, 3905insT |
| Disorders | | |
| CFTR-Related | CFTR | 4016insT, 4016insT |
| Disorders | | |
| CFTR-Related | CFTR | 4016insT, 4016insT |
| Disorders | | |
| CFTR-Related | CFTR | 394delTT, 394delTT |
| Disorders | | |
| CFTR-Related | CFTR | 394delTT, 394delTT |
| Disorders | | |
| CFTR-Related | CFTR | 405 + 1G → A |
| Disorders | | |
| CFTR-Related | CFTR | 405 + 3A → C |
| Disorders | | |
| CFTR-Related | CFTR | 444delA |
| Disorders | | |
| CFTR-Related | CFTR | 444delA |
| Disorders | | |
| CFTR-Related | CFTR | 3876delA, 3876delA |
| Disorders | | |
| CFTR-Related | CFTR | 3876delA, 3876delA |
| Disorders | | |
| CFTR-Related | CFTR | 457TAT → G |
| Disorders | | |
| CFTR-Related | CFTR | 457TAT → G |
| Disorders | | |
| CFTR-Related | CFTR | 3199del6, 3199del6 |
| Disorders | | |
| CFTR-Related | CFTR | 3199del6, 3199del6 |
| Disorders | | |
| CFTR-Related | CFTR | 406-1G → A |
| Disorders | | |
| CFTR-Related | CFTR | 663delT, 663delT |
| Disorders | | |
| CFTR-Related | CFTR | 663delT, 663delT |
| Disorders | | |
| CFTR-Related | CFTR | 935delA, 935delA |
| Disorders | | |
| CFTR-Related | CFTR | 935delA, 935delA |
| Disorders | | |
| CFTR-Related | CFTR | CFTR dele2,3 (21 kb) |
| Disorders | | |
| CFTR-Related | CFTR | CFTR dele2,3 (21 kb) |
| Disorders | | |
| CFTR-Related | CFTR | D1152H |
| Disorders | | |
| CFTR-Related | CFTR | E60X |
| Disorders | | |
| CFTR-Related | CFTR | E92X |
| Disorders | | |
| CFTR-Related | CFTR | F508C, rs1800093 |
| Disorders | | |
| CFTR-Related | CFTR | G178R |
| Disorders | | |
| CFTR-Related | CFTR | G330X |
| Disorders | | |
| CFTR-Related | CFTR | G480C |
| Disorders | | |
| CFTR-Related | CFTR | G542X |
| Disorders | | |
| CFTR-Related | CFTR | G551D |
| Disorders | | |
| CFTR-Related | CFTR | G622D |
| Disorders | | |
| CFTR-Related | CFTR | G85E |
| Disorders | | |
| CFTR-Related | CFTR | G91R |
| Disorders | | |
| CFTR-Related | CFTR | I148T, rs35516286 |
| Disorders | | |
| CFTR-Related | CFTR | I506V |
| Disorders | | |
| CFTR-Related | CFTR | IVS8-5T |
| Disorders | | |
| CFTR-Related | CFTR | IVS8-7T |
| Disorders | | |
| CFTR-Related | CFTR | IVS8-9T |
| Disorders | | |
| CFTR-Related | CFTR | K710X |
| Disorders | | |
| CFTR-Related | CFTR | L206W |
| Disorders | | |
| CFTR-Related | CFTR | M1101K, rs36210737 |
| Disorders | | |
| CFTR-Related | CFTR | N1303K |
| Disorders | | |
| CFTR-Related | CFTR | P574H |
| Disorders | | |
| CFTR-Related | CFTR | Q1238X |
| Disorders | | |
| CFTR-Related | CFTR | Q359K/T360K_wt |
| Disorders | | |
| CFTR-Related | CFTR | Q493X |
| Disorders | | |
| CFTR-Related | CFTR | Q552X |
| Disorders | | |
| CFTR-Related | CFTR | Q890X |
| Disorders | | |
| CFTR-Related | CFTR | R1066C |
| Disorders | | |
| CFTR-Related | CFTR | R1070Q |
| Disorders | | |
| CFTR-Related | CFTR | R1158X |
| Disorders | | |
| CFTR-Related | CFTR | R1162X |
| Disorders | | |
| CFTR-Related | CFTR | R117C |
| Disorders | | |
| CFTR-Related | CFTR | R117H |
| Disorders | | |
| CFTR-Related | CFTR | R1283M |
| Disorders | | |
| CFTR-Related | CFTR | R334W |
| Disorders | | |
| CFTR-Related | CFTR | R347H |
| Disorders | | |
| CFTR-Related | CFTR | R347P |
| Disorders | | |
| CFTR-Related | CFTR | R352Q |
| Disorders | | |
| CFTR-Related | CFTR | R553X |
| Disorders | | |
| CFTR-Related | CFTR | R560T |
| Disorders | | |
| CFTR-Related | CFTR | R709X |
| Disorders | | |
| CFTR-Related | CFTR | R75X |
| Disorders | | |
| CFTR-Related | CFTR | R764X |
| Disorders | | |
| CFTR-Related | CFTR | S1196X |
| Disorders | | |
| CFTR-Related | CFTR | S1235R, rs34911792 |
| Disorders | | |
| CFTR-Related | CFTR | S1251N |
| Disorders | | |
| CFTR-Related | CFTR | S1255X |
| Disorders | | |
| CFTR-Related | CFTR | S364P |
| Disorders | | |
| CFTR-Related | CFTR | S5491 |
| Disorders | | |
| CFTR-Related | CFTR | S549N |
| Disorders | | |
| CFTR-Related | CFTR | S549R |
| Disorders | | |
| CFTR-Related | CFTR | S549R |
| Disorders | | |
| CFTR-Related | CFTR | T338I |
| Disorders | | |
| CFTR-Related | CFTR | V520F |
| Disorders | | |
| CFTR-Related | CFTR | W1089X |
| Disorders | | |
| CFTR-Related | CFTR | W1204X |
| Disorders | | |
| CFTR-Related | CFTR | W1204X |
| Disorders | | |
| CFTR-Related | CFTR | W1282X |
| Disorders | | |
| CFTR-Related | CFTR | Y1092X |
| Disorders | | |
| CFTR-Related | CFTR | Y122X |
| Disorders | | |
| Choroideremia | CHM | c.1609 + 2dupT |
| Choroideremia | CHM | c.1609 + 2dupT |
| CLN3-Related | CLN3 | c.461_677del |
| Neuronal Ceroid- | | |
| Lipofuscinosis | | |
| CLN3-Related | CLN3 | c.461_677del |
| Neuronal Ceroid- | | |
| Lipofuscinosis | | |
| CLN3-Related | CLN3 | c.791_1056del |
| Neuronal Ceroid- | | |
| Lipofuscinosis | | |
| CLN3-Related | CLN3 | c.791_1056del |
| Neuronal Ceroid- | | |
| Lipofuscinosis | | |
| CLN5-Related | CLN5 | c.1175_1176delAT |
| Neuronal Ceroid- | | |
| Lipofuscinosis | | |
| CLN5-Related | CLN5 | c.1175_1176delAT |
| Neuronal Ceroid- | | |
| Lipofuscinosis | | |
| CLN5-Related | CLN5 | c.225G > A |
| Neuronal Ceroid- | | |
| Lipofuscinosis | | |
| CLN8-Related | CLN8 | c.70C > G |
| Neuronal Ceroid- | | |
| Lipofuscinosis | | |
| Cohen Syndrome | VPS13B | c.3348_3349delCT |
| Cohen Syndrome | VPS13B | c.3348_3349delCT |
| Congenital Cataracts, | CTDP1 | IVS6 + 389C > T |
| Facial Dysmorphism, | | |
| and Neuropathy | | |
| Congenital Disorder of | PMM2 | p.F119L |
| Glycosylation Ia | | |
| Congenital Disorder of | PMM2 | p.R141H |
| Glycosylation Ia | | |
| Congenital Disorder of | MPI | R295H, rs28928906 |
| Glycosylation Ib | | |
| Congenital Finnish | NPHS1 | c.121_122del |
| Nephrosis | | |
| Congenital Finnish | NPHS1 | c.121_122del |
| Nephrosis | | |
| Congenital Finnish | NPHS1 | c.3325C > T |
| Nephrosis | | |
| Crohn Disease | NOD2 | 3020 ins C |
| Crohn Disease | NOD2 | 3020 ins C |
| Crohn Disease | NOD2 | G908R, rs2066845 |
| Crohn Disease | NOD2 | R702W, rs2066844 |
| Cystinosis | CTNS | 1035insC |
| Cystinosis | CTNS | 1035insC |
| Cystinosis | CTNS | 537del21 |
| Cystinosis | CTNS | 537del21 |
| Cystinosis | CTNS | 57 kb deletion |
| Cystinosis | CTNS | 57 kb deletion |
| Cystinosis | CTNS | D205N |
| Cystinosis | CTNS | L158P |
| Cystinosis | CTNS | W138X |
| DFNA 9 (COCH) | COCH | P51S |
| Diabetes and Hearing | mtDNA | 3234A > G |
| Loss | | |
| Diabetes and Hearing | mtDNA | 3271T > C |
| Loss | | |
| Diabetes and Hearing | mtDNA | G8363A |
| Loss | | |
| Diabetes and Hearing | mtDNA | T14709C |
| Loss | | |
| Early-Onset Primary | TOR1A | 904_906delGAG |
| Dystonia (DYT1) | | |
| Early-Onset Primary | TOR1A | 904_906delGAG |
| Dystonia (DYT1) | | |
| Epidermolysis Bullosa | LAMB3 | 3024delT |
| Junctional, Herlitz- | | |
| Pearson Type | | |
| Epidermolysis Bullosa | LAMB3 | 3024delT |
| Junctional, Herlitz- | | |
| Pearson Type | | |
| Epidermolysis Bullosa | LAMB3 | p.Q243X |
| Junctional, Herlitz- | | |
| Pearson Type | | |
| Epidermolysis Bullosa | LAMB3 | R144X |
| Junctional, Herlitz- | | |
| Pearson Type | | |
| Epidermolysis Bullosa | LAMB3 | R42X |
| Junctional, Herlitz- | | |
| Pearson Type | | |
| Epidermolysis Bullosa | LAMB3 | R635X |
| Junctional, Herlitz- | | |
| Pearson Type | | |
| Epidermolysis Bullosa | LAMA3 | R650X |
| Junctional, Herlitz- | | |
| Pearson Type | | |
| Epidermolysis Bullosa | LAMC2 | R95X |
| Junctional, Herlitz- | | |
| Pearson Type | | |
| Factor V Leiden | F5 | H1299R |
| Thrombophilia | | |
| Factor V Leiden | F5 | R506Q, rs6025 |
| Thrombophilia | | |
| Factor V R2 Mutation | F5 | rs6027 |
| Thrombophilia | | |
| Factor XI Deficiency | F11 | E117X (576G > T) |
| Factor XI Deficiency | F11 | F283L (1074T > C) |
| Factor XI Deficiency | F11 | IVS14 + 1G > A |
| Factor XI Deficiency | F11 | IVS14del14 |
| Factor XI Deficiency | F11 | IVS14del14 |
| Factor XIII Deficiency | F13A1 | V34L, rs5985 |
| Familial Adenomatous | APC | I1307K, rs1801155 |
| Polyposis | | |
| Familial Dysautonomia | IKBKAP | 2507 + 6T > C |
| Familial Dysautonomia | IKBKAP | P914L |
| Familial Dysautonomia | IKBKAP | R696P |
| Familial | APOB | R3500Q, rs5742904 |
| Hypercholesterolemia | | |
| Type B | | |
| Familial | APOB | R3500W |
| Hypercholesterolemia | | |
| Type B | | |
| Familial | APOB | R3531C, rs12713559 |
| Hypercholesterolemia | | |
| Type B | | |
| Familial Mediterranean | MEFV | A744S (2230G > T) |
| Fever | | |
| Familial Mediterranean | MEFV | delI692 (del2076_2078) |
| Fever | | |
| Familial Mediterranean | MEFV | delI692 (del2076_2078) |
| Fever | | |
| Familial Mediterranean | MEFV | E148Q (442 G > C), rs3743930 |
| Fever | | |
| Familial Mediterranean | MEFV | E167D (501 G > C) |
| Fever | | |
| Familial Mediterranean | MEFV | F479L (1437 C > G) |
| Fever | | |
| Familial Mediterranean | MEFV | K695R (2084A > G) |
| Fever | | |
| Familial Mediterranean | MEFV | M680I (2040G > C) |
| Fever | | |
| Familial Mediterranean | MEFV | M694I (2082G > A), rs28940578 |
| Fever | | |
| Familial Mediterranean | MEFV | M694V (2080A > G) |
| Fever | | |
| Familial Mediterranean | MEFV | P369S (1105 C > T), rs11466023 |
| Fever | | |
| Familial Mediterranean | MEFV | R408Q (1223G > A), rs11466024 |
| Fever | | |
| Familial Mediterranean | MEFV | R653H (1958G > A) |
| Fever | | |
| Familial Mediterranean | MEFV | R761H (2282G > A) |
| Fever | | |
| Familial Mediterranean | MEFV | T267I (800 C > T) |
| Fever | | |
| Familial Mediterranean | MEFV | V726A (2177T > C), rs28940579 |
| Fever | | |
| FANCC-Related | FANCC | 322delG |
| Fanconi Anemia | | |
| FANCC-Related | FANCC | 322delG |
| Fanconi Anemia | | |
| FANCC-Related | FANCC | IVS4 + 4A > T (711 + 4A > T) |
| Fanconi Anemia | | |
| FANCC-Related | FANCC | Q13X (37C > T) |
| Fanconi Anemia | | |
| FANCC-Related | FANCC | R547X |
| Fanconi Anemia | | |
| FGFR1-Related | FGFR1 | P252R |
| Craniosynostosis | | |
| FGFR2-Related | FGFR2 | P253R |
| Craniosynostosis | | |
| FGFR2-Related | FGFR2 | S252W |
| Craniosynostosis | | |
| FGFR3-Related | FGFR3 | A391E, rs28931615 |
| Craniosynostosis | | |
| FGFR3-Related | FGFR3 | P250R, rs4647924 |
| Craniosynostosis | | |
| Free Sialic Acid | SLC17A5 | c.1007_1008delTA |
| Storage Disorders | | |
| Free Sialic Acid | SLC17A5 | c.1007_1008delTA |
| Storage Disorders | | |
| Free Sialic Acid | SLC17A5 | c.115C > T |
| Storage Disorders | | |
| Frontotemporal | MAPT | IVS10 + 16 |
| Dementia with | | |
| Parkinsonism-17 | | |
| Frontotemporal | MAPT | P301L |
| Dementia with | | |
| Parkinsonism-17 | | |
| Frontotemporal | MAPT | P301S |
| Dementia with | | |
| Parkinsonism-17 | | |
| Frontotemporal | MAPT | R406W |
| Dementia with | | |
| Parkinsonism-17 | | |
| Fumarase deficiency | FH | c.1431_1433dupAAA |
| Fumarase deficiency | FH | c.1431_1433dupAAA |
| Galactosemia | GALT | 5.0 Kb gene deletion |
| Galactosemia | GALT | 5.0 Kb gene deletion |
| Galactosemia | GALT | 5′ UTR-119del |
| Galactosemia | GALT | 5′ UTR-119del |
| Galactosemia | GALT | IVS2-2 A > G |
| Galactosemia | GALT | K285N |
| Galactosemia | GALT | L195P T > C |
| Galactosemia | GALT | L218L |
| Galactosemia | GALT | N314D, rs2070074 |
| Galactosemia | GALT | Phe171Ser |
| Galactosemia | GALT | Q169K |
| Galactosemia | GALT | Q188R |
| Galactosemia | GALT | S135L |
| Galactosemia | GALT | T138M C > T |
| Galactosemia | GALT | X380R |
| Galactosemia | GALT | Y209C A > G |
| Gaucher Disease | GBA | 1035insG |
| Gaucher Disease | GBA | 1035insG |
| Gaucher Disease | GBA | 84insG |
| Gaucher Disease | GBA | 84insG |
| Gaucher Disease | GBA | D409H, rs1064651 |
| Gaucher Disease | GBA | D409V |
| Gaucher Disease | GBA | IVS2(+1)G > A |
| Gaucher Disease | GBA | L444P (1448T > C), rs35095275 |
| Gaucher Disease | GBA | N370S |
| Gaucher Disease | GBA | R463C |
| Gaucher Disease | GBA | R463H |
| Gaucher Disease | GBA | R496H (1604G > A) |
| Gaucher Disease | GBA | V394L |
| GJB2-Related DFNA 3 | GJB2 | 167delT |
| Nonsyndromic Hearing | | |
| Loss and Deafness | | |
| GJB2-Related DFNA 3 | GJB2 | 167delT |
| Nonsyndromic Hearing | | |
| Loss and Deafness | | |
| GJB2-Related DFNA 3 | GJB2 | 235delC |
| Nonsyndromic Hearing | | |
| Loss and Deafness | | |
| GJB2-Related DFNA 3 | GJB2 | 235delC |
| Nonsyndromic Hearing | | |
| Loss and Deafness | | |
| GJB2-Related DFNA 3 | GJB2 | 35delG |
| Nonsyndromic Hearing | | |
| Loss and Deafness | | |
| GJB2-Related DFNA 3 | GJB2 | 35delG |
| Nonsyndromic Hearing | | |
| Loss and Deafness | | |
| GJB2-Related DFNA 3 | GJB2 | IVS1 + 1G > A |
| Nonsyndromic Hearing | | |
| Loss and Deafness | | |
| GJB2-Related DFNB 1 | GJB2 | 101delAG |
| Nonsyndromic Hearing | | |
| Loss and Deafness | | |
| GJB2-Related DFNB 1 | GJB2 | 313del14 |
| Nonsyndromic Hearing | | |
| Loss and Deafness | | |
| GJB2-Related DFNB 1 | GJB2 | 313del14 |
| Nonsyndromic Hearing | | |
| Loss and Deafness | | |
| GJB2-Related DFNB 1 | GJB2 | delE120 |
| Nonsyndromic Hearing | | |
| Loss and Deafness | | |
| GJB2-Related DFNB 1 | GJB2 | delE120 |
| Nonsyndromic Hearing | | |
| Loss and Deafness | | |
| GJB2-Related DFNB 1 | GJB2 | M34T, rs35887622 |
| Nonsyndromic Hearing | | |
| Loss and Deafness | | |
| GJB2-Related DFNB 1 | GJB2 | Q124X |
| Nonsyndromic Hearing | | |
| Loss and Deafness | | |
| GJB2-Related DFNB 1 | GJB2 | R184P |
| Nonsyndromic Hearing | | |
| Loss and Deafness | | |
| GJB2-Related DFNB 1 | GJB2 | V37I |
| Nonsyndromic Hearing | | |
| Loss and Deafness | | |
| GJB2-Related DFNB 1 | GJB2 | W24X |
| Nonsyndromic Hearing | | |
| Loss and Deafness | | |
| GJB2-Related DFNB 1 | GJB2 | W77R |
| Nonsyndromic Hearing | | |
| Loss and Deafness | | |
| GJB2-Related DFNB 1 | GJB2 | W77X |
| Nonsyndromic Hearing | | |
| Loss and Deafness | | |
| Glucose-6-Phosphate | G6PD | A335V |
| Dehydrogenase | | |
| Deficiency | | |
| Glucose-6-Phosphate | G6PD | R459L |
| Dehydrogenase | | |
| Deficiency | | |
| Glucose-6-Phosphate | G6PD | R459P |
| Dehydrogenase | | |
| Deficiency | | |
| Glucose-6-Phosphate | G6PD | rs1050828, rs1050828 |
| Dehydrogenase | | |
| Deficiency | | |
| Glucose-6-Phosphate | G6PD | rs1050829, rs1050829 |
| Dehydrogenase | | |
| Deficiency | | |
| Glucose-6-Phosphate | G6PD | rs5030868, rs5030868 |
| Dehydrogenase | | |
| Deficiency | | |
| Glutaricacidemia Type | GCDH | A421V |
| 1 | | |
| Glutaricacidemia Type | GCDH | R402W |
| 1 | | |
| Glycogen Storage | G6PC | 459insTA |
| Disease Type 1a | | |
| Glycogen Storage | G6PC | 459insTA |
| Disease Type 1a | | |
| Glycogen Storage | G6PC | 727G/T |
| Disease Type 1a | | |
| Glycogen Storage | G6PC | del F327 |
| Disease Type 1a | | |
| Glycogen Storage | G6PC | del F327 |
| Disease Type 1a | | |
| Glycogen Storage | G6PC | G188R |
| Disease Type 1a | | |
| Glycogen Storage | G6PC | G270V |
| Disease Type 1a | | |
| Glycogen Storage | G6PC | Q242X |
| Disease Type 1a | | |
| Glycogen Storage | G6PC | Q27fsdelC |
| Disease Type 1a | | |
| Glycogen Storage | G6PC | Q27fsdelC |
| Disease Type 1a | | |
| Glycogen Storage | G6PC | Q347X |
| Disease Type 1a | | |
| Glycogen Storage | G6PC | R83C |
| Disease Type 1a | | |
| Glycogen Storage | G6PC | R83H |
| Disease Type 1a | | |
| Glycogen Storage | G6PT1 | 1211delCT |
| Disease Type 1b | | |
| Glycogen Storage | G6PT1 | A367T |
| Disease Type 1b | | |
| Glycogen Storage | G6PT1 | G339C |
| Disease Type 1b | | |
| Glycogen Storage | G6PT1 | G339D |
| Disease Type 1b | | |
| Glycogen Storage | G6PT1 | W118R |
| Disease Type 1b | | |
| Glycogen Storage | GAA | Arg854X |
| Disease Type II | | |
| Glycogen Storage | GAA | Asp645Glu, rs28940868 |
| Disease Type II | | |
| Glycogen Storage | GAA | IVS1 (−13t > g) |
| Disease Type II | | |
| Glycogen Storage | AGL | 1484delT |
| Disease Type III | | |
| Glycogen Storage | AGL | 1484delT |
| Disease Type III | | |
| Glycogen Storage | AGL | 17delAG |
| Disease Type III | | |
| Glycogen Storage | AGL | 17delAG |
| Disease Type III | | |
| Glycogen Storage | AGL | Q6X |
| Disease Type III | | |
| Glycogen Storage | PYGM | G2045 |
| Disease Type V | | |
| Glycogen Storage | PYGM | K542T |
| Disease Type V | | |
| Glycogen Storage | PYGM | K542X |
| Disease Type V | | |
| Glycogen Storage | PYGM | R49X |
| Disease Type V | | |
| GNE-Related | GNE | M712T, rs28937594 |
| Myopathies | | |
| Gracile Syndrome | BCS1L | c.232A > G, rs28937590 |
| Hemoglobin S Beta- | HBB | c.19G > A |
| Thalassemia | | |
| Hemoglobin S Beta- | HBB | c.20A > T |
| Thalassemia | | |
| Hemoglobin S Beta- | HBB | c.79G > A |
| Thalassemia | | |
| Hemoglobin S Beta- | HBB | Hb CS |
| Thalassemia | | |
| Hemoglobin S Beta- | HBB | Hb D |
| Thalassemia | | |
| Hemoglobin S Beta- | HBB | Hb O |
| Thalassemia | | |
| Hereditary Fructose | ALDOB | A149P, rs1800546 |
| Intolerance | | |
| Hereditary Fructose | ALDOB | A174D |
| Intolerance | | |
| Hereditary Fructose | ALDOB | Delta4E4 |
| Intolerance | | |
| Hereditary Fructose | ALDOB | Delta4E4 |
| Intolerance | | |
| Hereditary Fructose | ALDOB | N334K |
| Intolerance | | |
| Hereditary Fructose | ALDOB | Y203X |
| Intolerance | | |
| Hereditary Pancreatitis | PRSS1 | A16V |
| Hereditary Pancreatitis | SPINK1 | M1T |
| Hereditary Pancreatitis | PRSS1 | N29I |
| Hereditary Pancreatitis | SPINK1 | N34S, rs17107315 |
| Hereditary Pancreatitis | PRSS1 | R122C |
| Hereditary Pancreatitis | PRSS1 | R122H |
| Hereditary Thymine- | DPYD | rs3918290 |
| Uraciluria | | |
| Hexosaminidase A | HEXA | 1278insTATC |
| Deficiency | | |
| Hexosaminidase A | HEXA | 1278insTATC |
| Deficiency | | |
| Hexosaminidase A | HEXA | G269S (805G > A) |
| Deficiency | | |
| Hexosaminidase A | HEXA | IVS12 + 1G > C |
| Deficiency | | |
| Hexosaminidase A | HEXA | IVS7 + 1G > A |
| Deficiency | | |
| Hexosaminidase A | HEXA | IVS9 + 1G > A |
| Deficiency | | |
| Hexosaminidase A | HEXA | R178C |
| Deficiency | | |
| Hexosaminidase A | HEXA | R178H |
| Deficiency | | |
| Hexosaminidase A | HEXA | R247W (739C > T) |
| Deficiency | | |
| Hexosaminidase A | HEXA | R249W (745C > T) |
| Deficiency | | |
| HFE-Associated | HFE | E168Q |
| Hereditary | | |
| Hemochromatosis | | |
| HFE-Associated | HFE | E168X |
| Hereditary | | |
| Hemochromatosis | | |
| HFE-Associated | HFE | HM971246, H63H |
| Hereditary | | |
| Hemochromatosis | | |
| HFE-Associated | HFE | P160delC |
| Hereditary | | |
| Hemochromatosis | | |
| HFE-Associated | HFE | P160delC |
| Hereditary | | |
| Hemochromatosis | | |
| HFE-Associated | HFE | Q127H, rs28934595 |
| Hereditary | | |
| Hemochromatosis | | |
| HFE-Associated | HFE | Q283P |
| Hereditary | | |
| Hemochromatosis | | |
| HFE-Associated | HFE | rs1799945, rs1799945 |
| Hereditary | | |
| Hemochromatosis | | |
| HFE-Associated | HFE | rs1800562, rs1800562 |
| Hereditary | | |
| Hemochromatosis | | |
| HFE-Associated | HFE | rs1800730, rs1800730 |
| Hereditary | | |
| Hemochromatosis | | |
| HFE-Associated | HFE | V53M |
| Hereditary | | |
| Hemochromatosis | | |
| HFE-Associated | HFE | V59M |
| Hereditary | | |
| Hemochromatosis | | |
| HFE-Associated | HFE | W169X |
| Hereditary | | |
| Hemochromatosis | | |
| Hidrotic Ectodermal | GJB6 | A88V, rs28937872 |
| Dysplasia 2 | | |
| Hidrotic Ectodermal | GJB6 | G11R |
| Dysplasia 2 | | |
| Hidrotic Ectodermal | GJB6 | V37E |
| Dysplasia 2 | | |
| Homocystinuria | CBS | G307S 919G → A |
| Caused by | | |
| Cystathionine Beta- | | |
| Synthase Deficiency | | |
| Homocystinuria | CBS | I278T 833T → C, rs5742905 |
| Caused by | | |
| Cystathionine Beta- | | |
| Synthase Deficiency | | |
| Hyperkalemic Periodic | SCN4A | I693T |
| Paralysis Type 1 | | |
| Hyperkalemic Periodic | SCN4A | L689I |
| Paralysis Type 1 | | |
| Hyperkalemic Periodic | SCN4A | L689V |
| Paralysis Type 1 | | |
| Hyperkalemic Periodic | SCN4A | M1360V |
| Paralysis Type 1 | | |
| Hyperkalemic Periodic | SCN4A | M1592V |
| Paralysis Type 1 | | |
| Hyperkalemic Periodic | SCN4A | p.A1156T |
| Paralysis Type 1 | | |
| Hyperkalemic Periodic | SCN4A | p.M1370V |
| Paralysis Type 1 | | |
| Hyperkalemic Periodic | SCN4A | p.R1448C |
| Paralysis Type 1 | | |
| Hyperkalemic Periodic | SCN4A | p.T1313M |
| Paralysis Type 1 | | |
| Hyperkalemic Periodic | SCN4A | R675G |
| Paralysis Type 1 | | |
| Hyperkalemic Periodic | SCN4A | R675Q |
| Paralysis Type 1 | | |
| Hyperkalemic Periodic | SCN4A | R675W |
| Paralysis Type 1 | | |
| Hyperkalemic Periodic | SCN4A | T704M |
| Paralysis Type 1 | | |
| Hyperkalemic Periodic | SCN4A | V781I |
| Paralysis Type 1 | | |
| Hyperomithinemia- | | |
| Hyperammonemia- | SLC25A15 | F188del |
| Homocitrullinuria | | |
| Syndrome | | |
| Hyperomithinemia- | SLC25A15 | F188del |
| Hyperammonemia- | | |
| Homocitrullinuria | | |
| Syndrome | | |
| Hyperoxaluria, | AGXT | 33insC |
| Primary, Type 1 | | |
| Hyperoxaluria, | AGXT | 33insC |
| Primary, Type 1 | | |
| Hyperoxaluria, | AGXT | F152I |
| Primary, Type 1 | | |
| Hyperoxaluria, | AGXT | G170R |
| Primary, Type 1 | | |
| Hyperoxaluria, | AGXT | I244T |
| Primary, Type 1 | | |
| Hyperoxaluria, | GRHPR | 103delG |
| Primary, Type 2 | | |
| Hyperoxaluria, | GRHPR | 103delG |
| Primary, Type 2 | | |
| Hypochondroplasia | FGFR3 | Asn328Ile |
| Hypochondroplasia | FGFR3 | I538V |
| Hypochondroplasia | FGFR3 | K650M |
| Hypochondroplasia | FGFR3 | K650N 1950G > T |
| Hypochondroplasia | FGFR3 | K650Q |
| Hypochondroplasia | FGFR3 | N540K 1620C > A |
| Hypochondroplasia | FGFR3 | N540S |
| Hypochondroplasia | FGFR3 | N540T |
| Hypokalemic Periodic | CACNA1S | R528G |
| Paralysis Type 1 | | |
| Hypokalemic Periodic | CACNA1S | R528H |
| Paralysis Type 1 | | |
| Hypokalemic Periodic | CACNA1S | rs28930068, rs28930068 |
| Paralysis Type 1 | | |
| Hypokalemic Periodic | CACNA1S | rs28930069, rs28930069 |
| Paralysis Type 1 | | |
| Hypokalemic Periodic | SCN4A | R669H |
| Paralysis Type 2 | | |
| Hypokalemic Periodic | SCN4A | R672C |
| Paralysis Type 2 | | |
| Hypokalemic Periodic | SCN4A | R672G |
| Paralysis Type 2 | | |
| Hypokalemic Periodic | SCN4A | R672H |
| Paralysis Type 2 | | |
| Hypokalemic Periodic | SCN4A | R672S |
| Paralysis Type 2 | | |
| Hypophosphatasia | ALPL | Asp361Val |
| Hypophosphatasia | ALPL | c.1559delT |
| Hypophosphatasia | ALPL | c.1559delT |
| Hypophosphatasia | ALPL | E174K |
| Hypophosphatasia | ALPL | G317D |
| Hypophosphatasia | ALPL | Phe310Leu |
| Isovaleric Acidemia | IVD | A282V |
| Isovaleric Acidemia | IVD | rs28940889 |
| Krabbe Disease | GALC | EX11-17DEL |
| Krabbe Disease | GALC | EX11-17DEL |
| Krabbe Disease | GALC | G270D |
| Krabbe Disease | GALC | rs1805078, rs1805078 |
| Krabbe Disease | GALC | rs398607 |
| Leber Hereditary Optic | mtDNA | 14484T > C |
| Neuropathy | | |
| Leber Hereditary Optic | mtDNA | 15257G > A |
| Neuropathy | | |
| Leber Hereditary Optic | mtDNA | G14459A |
| Neuropathy | | |
| Leber Hereditary Optic | mtDNA | G3460A |
| Neuropathy | | |
| Leber Hereditary Optic | mtDNA | m.11778G > A |
| Neuropathy | | |
| Leber Hereditary Optic | mtDNA | m.13708G > A |
| Neuropathy | | |
| Leber Hereditary Optic | mtDNA | m.15812G > A |
| Neuropathy | | |
| Leber Hereditary Optic | mtDNA | m.3394T > C |
| Neuropathy | | |
| Leber Hereditary Optic | mtDNA | m.4216T > C |
| Neuropathy | | |
| Leber Hereditary Optic | mtDNA | m.4917A > G |
| Neuropathy | | |
| Leigh Syndrome, | LRPPRC | A354V |
| French-Canadian Type | | |
| LGMD2I | FKRP | L276I, rs28937900 |
| Long Chain 3- | HADHA | E474Q c.1528G > C |
| Hydroxyacyl-CoA | | |
| Dehydrogenase | | |
| Deficiency | | |
| Long Chain 3- | HADHA | Q342X 1132C > T |
| Hydroxyacyl-CoA | | |
| Dehydrogenase | | |
| Deficiency | | |
| Maple Syrup Urine | BCKDHA | Y438N |
| Disease Type 1A | | |
| Maple Syrup Urine | BCKDHB | E372X |
| Disease Type 1B | | |
| Maple Syrup Urine | BCKDHB | G278S |
| Disease Type 1B | | |
| Maple Syrup Urine | BCKDHB | R183P |
| Disease Type 1B | | |
| McCune-Albright | GNAS | R201C |
| Syndrome | | |
| McCune-Albright | GNAS | R201G |
| Syndrome | | |
| McCune-Albright | GNAS | R201H |
| Syndrome | | |
| Medium Chain Acyl- | ACADM | 244insT |
| Coenzyme A | | |
| Dehydrogenase | | |
| Deficiency | | |
| Medium Chain Acyl- | ACADM | 244insT |
| Coenzyme A | | |
| Dehydrogenase | | |
| Deficiency | | |
| Medium Chain Acyl- | ACADM | 250C > T |
| Coenzyme A | | |
| Dehydrogenase | | |
| Deficiency | | |
| Medium Chain Acyl- | ACADM | 583G > A |
| Coenzyme A | | |
| Dehydrogenase | | |
| Deficiency | | |
| Medium Chain Acyl- | ACADM | 616C > T |
| Coenzyme A | | |
| Dehydrogenase | | |
| Deficiency | | |
| Medium Chain Acyl- | ACADM | 617G > A |
| Coenzyme A | | |
| Dehydrogenase | | |
| Deficiency | | |
| Medium Chain Acyl- | ACADM | 799G > A |
| Coenzyme A | | |
| Dehydrogenase | | |
| Deficiency | | |
| Medium Chain Acyl- | ACADM | K304E |
| Coenzyme A | | |
| Dehydrogenase | | |
| Deficiency | | |
| Medium Chain Acyl- | ACADM | Y42H |
| Coenzyme A | | |
| Dehydrogenase | | |
| Deficiency | | |
| Megalencephalic | MLC1 | 135insC |
| Leukoencephalopathy | | |
| with Subcortical Cysts | | |
| Megalencephalic | MLC1 | 135insC |
| Leukoencephalopathy | | |
| with Subcortical Cysts | | |
| MELAS | mtDNA | 3243A > G |
| MELAS | mtDNA | 3250T > C |
| MELAS | mtDNA | 3252A > G |
| MELAS | mtDNA | A12770G |
| MELAS | mtDNA | C3256T |
| MELAS | mtDNA | G13513A |
| MELAS | mtDNA | T3291C |
| MELAS | mtDNA | T8356C |
| MELAS | mtDNA | T9957C |
| MERRF | mtDNA | 8361G > A |
| MERRF | mtDNA | A8296G |
| MERRF | mtDNA | m.8344A > G |
| Metachromatic | ARSA | c.459 + 1G > A |
| Leukodystrophy | | |
| Metachromatic | ARSA | p.P426L, rs28940893 |
| Leukodystrophy | | |
| Metachromatic | ARSA | p.T274M |
| Leukodystrophy | | |
| Metachromatic | ARSA | P377L |
| Leukodystrophy | | |
| Mitochondrial | mtDNA | A3260T |
| Cardiomyopathy | | |
| Mitochondrial | mtDNA | A4300G |
| Cardiomyopathy | | |
| Mitochondrial | | |
| Cardiomyopathy | mtDNA | C3303T |
| Mitochondrial | | |
| Cardiomyopathy | mtDNA | T9997C |
| Mitochondrial DNA- | mtDNA | 5537insT |
| Associated Leigh | | |
| Syndrome and NARP | | |
| Mitochondrial DNA- | mtDNA | 5537insT |
| Associated Leigh | | |
| Syndrome and NARP | | |
| Mitochondrial DNA- | mtDNA | 8993T > C |
| Associated Leigh | | |
| Syndrome and NARP | | |
| Mitochondrial DNA- | mtDNA | 8993T > G |
| Associated Leigh | | |
| Syndrome and NARP | | |
| Mitochondrial DNA- | mtDNA | C11777A |
| Associated Leigh | | |
| Syndrome and NARP | | |
| Mitochondrial DNA- | mtDNA | T10158C |
| Associated Leigh | | |
| Syndrome and NARP | | |
| Mitochondrial DNA- | mtDNA | T10191C |
| Associated Leigh | | |
| Syndrome and NARP | | |
| Mitochondrial DNA- | mtDNA | T8851C |
| Associated Leigh | | |
| Syndrome and NARP | | |
| Mitochondrial DNA- | mtDNA | T9176C |
| Associated Leigh | | |
| Syndrome and NARP | | |
| Mitochondrial DNA- | mtDNA | T9176G |
| Associated Leigh | | |
| Syndrome and NARP | | |
| MTHFR Deficiency | MTHFR | 1298A > C |
| MTHFR Deficiency | MTHFR | rs1801133, rs1801133 |
| MTRNR1-Related | mtDNA | 1095T > C |
| Hearing Loss and | | |
| Deafness | | |
| MTRNR1-Related | mtDNA | 1494C > T |
| Hearing Loss and | | |
| Deafness | | |
| MTRNR1-Related | mtDNA | 1555A > G |
| Hearing Loss and | | |
| Deafness | | |
| MTRNR1-Related | mtDNA | 961T > G |
| Hearing Loss and | | |
| Deafness | | |
| MTRNR1-Related | mtDNA | A7445G |
| Hearing Loss and | | |
| Deafness | | |
| MTTS1-Related | mtDNA | 7443A > G |
| Hearing Loss and | | |
| Deafness | | |
| MTTS1-Related | mtDNA | 7444G > A |
| Hearing Loss and | | |
| Deafness | | |
| MTTS1-Related | mtDNA | 7472insC |
| Hearing Loss and | | |
| Deafness | | |
| MTTS1-Related | mtDNA | 7472insC |
| Hearing Loss and | | |
| Deafness | | |
| MTTS1-Related | mtDNA | 7510T > C |
| Hearing Loss and | | |
| Deafness | | |
| MTTS1-Related | mtDNA | 7511T > C |
| Hearing Loss and | | |
| Deafness | | |
| MTTS1-Related | mtDNA | 7512T > C |
| Hearing Loss and | | |
| Deafness | | |
| Mucolipidosis IV | MCOLN1 | delEx1 3 Ex7 (511 > 6944del) |
| Mucolipidosis IV | MCOLN1 | delEx1 3 Ex7 (511 > 6944del) |
| Mucolipidosis IV | MCOLN1 | IVS-2A > G |
| Mucopolysaccharidosis | IDUA | c.46_57del |
| Type I | | |
| Mucopolysaccharidosis | IDUA | c.46_57del |
| Type I | | |
| Mucopolysaccharidosis | IDUA | p.A327P |
| Type I | | |
| Mucopolysaccharidosis | IDUA | p.P533R |
| Type I | | |
| Mucopolysaccharidosis | IDUA | Q70X |
| Type I | | |
| Mucopolysaccharidosis | IDUA | W402X |
| Type I | | |
| Mucopolysaccharidosis | SGSH | p.R245H |
| Type IIIA | | |
| Mucopolysaccharidosis | SGSH | p.R74C |
| Type IIIA | | |
| Mucopolysaccharidosis | SGSH | p.S66W |
| Type IIIA | | |
| Mucopolysaccharidosis | GUSB | p.D152N |
| Type VII | | |
| Multiple Endocrine | RET | 2047T > A |
| Neoplasia Type 2 | | |
| Multiple Endocrine | RET | 2047T > A |
| Neoplasia Type 2 | | |
| Multiple Endocrine | RET | 2047T > C |
| Neoplasia Type 2 | | |
| Multiple Endocrine | RET | 2047T > G |
| Neoplasia Type 2 | | |
| Multiple Endocrine | RET | 2048G > A |
| Neoplasia Type 2 | | |
| Multiple Endocrine | RET | A883F 2647 G > T |
| Neoplasia Type 2 | | |
| Multiple Endocrine | RET | Glu768Asp G > C |
| Neoplasia Type 2 | | |
| Multiple Endocrine | RET | M918T |
| Neoplasia Type 2 | | |
| Muscle-Eye-Brain | POMGNT1 | c.1539 + 1G > A |
| Disease | | |
| MYH-Associated | MUTYH | c.1376C > A |
| Polyposis | | |
| MYH-Associated | MUTYH | c.494A > G, rs34612342 |
| Polyposis | | |
| MYH-Associated | GENE_SYMBOL_ | rs36053993, rs36053993 |
| Polyposis | TBD | |
| Niemann-Pick Disease | SMPD1 | c.990delC |
| Due to | | |
| Sphingomyelinase | | |
| Deficiency | | |
| Niemann-Pick Disease | SMPD1 | c.990delC |
| Due to | | |
| Sphingomyelinase | | |
| Deficiency | | |
| Niemann-Pick Disease | SMPD1 | fsP330 |
| Due to | | |
| Sphingomyelinase | | |
| Deficiency | | |
| Niemann-Pick Disease | SMPD1 | fsP330 |
| Due to | | |
| Sphingomyelinase | | |
| Deficiency | | |
| Niemann-Pick Disease | SMPD1 | L302P |
| Due to | | |
| Sphingomyelinase | | |
| Deficiency | | |
| Niemann-Pick Disease | SMPD1 | R496L |
| Due to | | |
| Sphingomyelinase | | |
| Deficiency | | |
| Niemann-Pick Disease | SMPD1 | R608del |
| Due to | | |
| Sphingomyelinase | | |
| Deficiency | | |
| Niemann-Pick Disease | NPC1 | 11061T |
| Type C1 | | |
| Nijmegen Breakage | NBN | 657del5 |
| Syndrome | | |
| Nijmegen Breakage | NBN | 657del5 |
| Syndrome | | |
| Pallister-Hall | GLI3 | 2012delG |
| Syndrome | | |
| Pallister-Hall | GLI3 | 2012delG |
| Syndrome | | |
| Pallister-Hall | GLI3 | 2023delG |
| Syndrome | | |
| Pallister-Hall | GLI3 | 2023delG |
| Syndrome | | |
| Pendred Syndrome | SLC26A4 | 1197delT |
| Pendred Syndrome | SLC26A4 | 1197delT |
| Pendred Syndrome | SLC26A4 | E384G |
| Pendred Syndrome | SLC26A4 | IV58 + 1(G → A) |
| Pendred Syndrome | SLC26A4 | L236P |
| Pendred Syndrome | SLC26A4 | T416P |
| Peroxisomal | | |
| Bifunctional Enzyme | HSD17B4 | c.302 + 1G > C |
| Deficiency | | |
| Peroxisomal | | |
| Bifunctional Enzyme | HSD17B4 | c.303 − 1G > A |
| Deficiency | | |
| Pervasive | NLGN4X | D396X |
| Developmental | | |
| Disorders | | |
| Pervasive | NLGN4X | D396X |
| Developmental | | |
| Disorders | | |
| Pervasive | NLGN4X | NLGN4X:1253delAG |
| Developmental | | |
| Disorders | | |
| Pervasive | NLGN4X | NLGN4X:1253delAG |
| Developmental | | |
| Disorders | | |
| Pervasive | NLGN3 | R451C |
| Developmental | | |
| Disorders | | |
| Phenylalanine | PAH | G272X |
| Hydroxylase | | |
| Deficiency | | |
| Phenylalanine | PAH | I65T |
| Hydroxylase | | |
| Deficiency | | |
| Phenylalanine | PAH | IVS12 + 1G > T |
| Hydroxylase | | |
| Deficiency | | |
| Phenylalanine | PAH | L48S, rs5030841 |
| Hydroxylase | | |
| Deficiency | | |
| Phenylalanine | PAH | R158Q, rs5030843 |
| Hydroxylase | | |
| Deficiency | | |
| Phenylalanine | PAH | R252W, rs5030847 |
| Hydroxylase | | |
| Deficiency | | |
| Phenylalanine | PAH | R261Q, rs5030849 |
| Hydroxylase | | |
| Deficiency | | |
| Phenylalanine | PAH | R408Q, rs5030859 |
| Hydroxylase | | |
| Deficiency | | |
| Phenylalanine | PAH | R408W, rs5030858 |
| Hydroxylase | | |
| Deficiency | | |
| Phenylalanine | PAH | rs5030855 |
| Hydroxylase | | |
| Deficiency | | |
| Phenylalanine | PAH | rs5030861 |
| Hydroxylase | | |
| Deficiency | | |
| Phenylalanine | PAH | Y414C, rs5030860 |
| Hydroxylase | | |
| Deficiency | | |
| Plasminogen Activator | SERPINE1 | −844 G > A |
| Inhibitor I | | |
| Plasminogen Activator | SERPINE1 | 4G/5G |
| Inhibitor I | | |
| Polycystic Kidney | PKHD1 | c.10412T > G |
| Disease, Autosomal | | |
| Recessive | | |
| Polycystic Kidney | PKHD1 | c.107C > T |
| Disease, Autosomal | | |
| Recessive | | |
| Polycystic Kidney | PKHD1 | c.1486C > T |
| Disease, Autosomal | | |
| Recessive | | |
| Polycystic Kidney | PKHD1 | c.5895dupA |
| Disease, Autosomal | | |
| Recessive | | |
| Polycystic Kidney | PKHD1 | c.5895dupA |
| Disease, Autosomal | | |
| Recessive | | |
| Polycystic Kidney | PKHD1 | c.9689delA |
| Disease, Autosomal | | |
| Recessive | | |
| Polycystic Kidney | PKHD1 | c.9689delA |
| Disease, Autosomal | | |
| Recessive | | |
| PPT1-Related Neuronal | PPT1 | c.364A > T |
| Ceroid-Lipofuscinosis | | |
| PPT1-Related Neuronal | PPT1 | p.L10X |
| Ceroid-Lipofuscinosis | | |
| PPT1-Related Neuronal | PPT1 | p.R151X |
| Ceroid-Lipofuscinosis | | |
| PPT1-Related Neuronal | PPT1 | T75P |
| Ceroid-Lipofuscinosis | | |
| PROP1-related | PROP1 | 301-302delAG |
| pituitary hormome | | |
| deficiency | | |
| PROP1-related | PROP1 | 301-302delAG |
| pituitary hormome | | |
| deficiency | | |
| Prothrombin | F2 | rs1799963 |
| Thrombophilia | | |
| Prothrombin | F2 | rs6025, rs6025 |
| Thrombophilia | | |
| Pseudovitamin D | CYP27B1 | 7 bp duplication in exon8 |
| Deficiency Rickets | | |
| Pseudovitamin D | CYP27B1 | 7 bp duplication in exon8 |
| Deficiency Rickets | | |
| Pseudovitamin D | CYP27B1 | 958delG |
| Deficiency Rickets | | |
| Pseudovitamin D | CYP27B1 | 958delG |
| Deficiency Rickets | | |
| Rett Syndrome | MECP2 | 806delG |
| Rett Syndrome | MECP2 | 806delG |
| Rett Syndrome | MECP2 | A140V, rs28934908 |
| Rett Syndrome | MECP2 | P152R |
| Rett Syndrome | MECP2 | P225R |
| Rett Syndrome | MECP2 | R106W, rs28934907 |
| Rett Syndrome | MECP2 | R133C |
| Rett Syndrome | MECP2 | R168X |
| Rett Syndrome | MECP2 | R255X |
| Rett Syndrome | MECP2 | R270X |
| Rett Syndrome | MECP2 | R294X |
| Rett Syndrome | MECP2 | R306C, rs28935468 |
| Rett Syndrome | MECP2 | S134C |
| Rett Syndrome | MECP2 | T158M, rs28934906 |
| Rhizomelic | | |
| Chondrodysplasia | PEX7 | P.A218V |
| Punctata Type 1 | | |
| Rhizomelic | | |
| Chondrodysplasia | PEX7 | P.G217R |
| Punctata Type 1 | | |
| Rhizomelic | | |
| Chondrodysplasia | PEX7 | p.L292X, rs1805137 |
| Punctata Type 1 | | |
| Short Chain Acyl-CoA | ACADS | c.511C > T, rs1800556 |
| Dehydrogenase | | |
| Deficiency | | |
| Short Chain Acyl-CoA | ACADS | c.625G > A |
| Dehydrogenase | | |
| Deficiency | | |
| Short Chain Acyl-CoA | ACADS | R107C |
| Dehydrogenase | | |
| Deficiency | | |
| Shwachman-Diamond | SBDS | 183_184TA > CT |
| Syndrome | | |
| Shwachman-Diamond | SBDS | 183_184TA > CT |
| Syndrome | | |
| Shwachman-Diamond | SBDS | 258 + 2T > C |
| Syndrome | | |
| Sjogren-Larsson | ALDH3A2 | c.943C > T |
| Syndrome | | |
| Smith-Lemli-Opitz | DHCR7 | C380Y |
| Syndrome | | |
| Smith-Lemli-Opitz | DHCR7 | IVS8-1G > C |
| Syndrome | | |
| Smith-Lemli-Opitz | DHCR7 | L109P |
| Syndrome | | |
| Smith-Lemli-Opitz | DHCR7 | L157P |
| Syndrome | | |
| Smith-Lemli-Opitz | DHCR7 | R352Q |
| Syndrome | | |
| Smith-Lemli-Opitz | DHCR7 | R352W |
| Syndrome | | |
| Smith-Lemli-Opitz | DHCR7 | R404C |
| Syndrome | | |
| Smith-Lemli-Opitz | DHCR7 | R446Q |
| Syndrome | | |
| Smith-Lemli-Opitz | DHCR7 | T93M |
| Syndrome | | |
| Smith-Lemli-Opitz | DHCR7 | V326L |
| Syndrome | | |
| Smith-Lemli-Opitz | DHCR7 | W151X |
| Syndrome | | |
| Smith-Lemli-Opitz | DHCR7 | W151X |
| Syndrome | | |
| Spastic Paraplegia 13 | HSPD1 | V72I |
| Sulfate Transporter- | SLC26A2 | 340delV |
| Related | | |
| Osteochondrodysplasia | | |
| Sulfate Transporter- | SLC26A2 | 340delV |
| Related | | |
| Osteochondrodysplasia | | |
| Sulfate Transporter- | SLC26A2 | c.837C > T |
| Related | | |
| Osteochondrodysplasia | | |
| Sulfate Transporter- | SLC26A2 | C653S |
| Related | | |
| Osteochondrodysplasia | | |
| Sulfate Transporter- | SLC26A2 | IVS1 + 2T > C |
| Related | | |
| Osteochondrodysplasia | | |
| Sulfate Transporter- | SLC26A2 | R178X |
| Related | | |
| Osteochondrodysplasia | | |
| TFR2-Related | TFR2 | AVAQ594-597del |
| Hereditary | | |
| Hemochromatosis | | |
| TFR2-Related | TFR2 | AVAQ594-597del |
| Hereditary | | |
| Hemochromatosis | | |
| TFR2-Related | TFR2 | E60X |
| Hereditary | | |
| Hemochromatosis | | |
| TFR2-Related | TFR2 | E60X |
| Hereditary | | |
| Hemochromatosis | | |
| TFR2-Related | TFR2 | M172K |
| Hereditary | | |
| Hemochromatosis | | |
| TFR2-Related | TFR2 | Y250X |
| Hereditary | | |
| Hemochromatosis | | |
| Thanatophoric | FGFR3 | G370C |
| Dysplasia | | |
| Thanatophoric | FGFR3 | K650E |
| Dysplasia | | |
| Thanatophoric | FGFR3 | R248C |
| Dysplasia | | |
| Thanatophoric | FGFR3 | S249C |
| Dysplasia | | |
| Thanatophoric | FGFR3 | S371C |
| Dysplasia | | |
| Thanatophoric | FGFR3 | X807C A > T |
| Dysplasia | | |
| Thanatophoric | FGFR3 | X807G |
| Dysplasia | | |
| Thanatophoric | FGFR3 | X807L |
| Dysplasia | | |
| Thanatophoric | FGFR3 | X807R |
| Dysplasia | | |
| Thanatophoric | FGFR3 | X807S |
| Dysplasia | | |
| Thanatophoric | FGFR3 | X807W |
| Dysplasia | | |
| Thanatophoric | FGFR3 | Y373C |
| Dysplasia | | |
| TPP1-Related Neuronal | TPP1 | c.509-1G > A |
| Ceroid-Lipofuscinosis | | |
| TPP1-Related Neuronal | TPP1 | c.509-1G > C |
| Ceroid-Lipofuscinosis | | |
| TPP1-Related Neuronal | TPP1 | G284V |
| Ceroid-Lipofuscinosis | | |
| TPP1-Related Neuronal | TPP1 | p.R208X |
| Ceroid-Lipofuscinosis | | |
| Transthyretin | TTR | c.148G > A |
| Amyloidosis | | |
| Tyrosine Hydroxylase- | TH | L205P |
| Deficient DRD | | |
| Tyrosine Hydroxylase- | TH | R202H |
| Deficient DRD | | |
| Tyrosinemia Type I | FAH | E357X |
| Tyrosinemia Type I | FAH | IVS12 + 5 G > A |
| Tyrosinemia Type I | FAH | IVS7-6 T > G |
| Tyrosinemia Type I | FAH | IVS8-1G > C |
| Tyrosinemia Type I | FAH | p.W262X |
| Tyrosinemia Type I | FAH | P261L |
| Tyrosinemia Type I | FAH | Q64H |
| Wilson Disease | ATP7B | 1340del4 |
| Wilson Disease | ATP7B | 3402delC |
| Wilson Disease | ATP7B | 3402delC |
| Wilson Disease | ATP7B | H1069Q |
| Wilson Disease | ATP7B | R778G |
| Wilson Disease | ATP7B | W779X |
| Wilson Disease | ATP7B | W779X |
| X-Linked Juvenile | RS1 | E72K |
| Retinoschisis | | |
| X-Linked Juvenile | RS1 | G109R |
| Retinoschisis | | |
| X-Linked Juvenile | RS1 | G74V |
| Retinoschisis | | |
| Zellweger Syndrome | PEX1 | c.2097_2098insT |
| Spectrum | | |
| Zellweger Syndrome | PEX1 | c.2097_2098insT |
| Spectrum | | |
| Zellweger Syndrome | PEX1 | c.2916delA |
| Spectrum | | |
| Zellweger Syndrome | PEX1 | c.2916delA |
| Spectrum | | |
| Zellweger Syndrome | PEX1 | p.G843D |
| Spectrum | | |

TABLE 2
Genes in which mutations may be associated with disease

| Disease/ | |
|---|---|
| Disorder | Gene(s) |
| Neoplasia | PTEN; ATM; ATR; EGFR; ERBB2; ERBB3; ERBB4; |
| | Notch1; Notch2; Notch3; Notch4; AKT; AKT2: AKT3; |
| | HIF; HIF1a; HIF3a; Met; HRG; Bcl2; PPAR alpha; |
| | PPAR gamma; WT1 (Wilms Tumor); FGF Receptor |
| | Family members (5 members: 1, 2, 3, 4, 5); CDKN2a; |
| | APC; RB (retinoblastoma); MEN1; VHL; BRCA1; |
| | BRCA2; AR (Androgen Receptor); TSG101; IGF; IGF |
| | Receptor; Igf1 (4 variants); Igf2 (3 variants); Igf |
| | 1Receptor; Igf 2 Receptor; Bax; Bcl2; caspases family |
| | (9 members: 1, 2, 3, 4, 6, 7, 8, 9, 12); Kras; Apc |
| Age-related | Abcr; Ccl2; Cc2; cp (ceruloplasmin); Timp3; cathepsinD; |
| Macular | Vldir; Ccr2 |
| Degeneration | |
| Schizophrenia | Neuregulin1 (Nrg1); Erb4 (receptor for Neuregulin); |
| | Complexin1 (Cplx1); Tph1 Tryptophan hydroxylase; Tph2 |
| | Tryptophan hydroxylase 2; Neurexin 1; GSK3; GSK3a; |
| | GSK3b |
| Trinucleotide | HTT (Huntington's Dx); SRMA/SMAX1/AR. (Kennedy's |
| Repeat | Dx); FXN/X25 (Friedrich's Ataxia); ATX3 (Machado |
| Disorders | Joseph's Dx); ATXN1 and ATXN2 (spinocerebellar |
| | ataxias); DMPK (myotonic dystrophy); Atrophin-1 and |
| | Ant1 (DRPLA Dx); CBP (Creb-BP - global instability); |
| | VLDLR (Alzheimer's); Atxn7; Atxn10 |
| Fragile X | FMER2; FXR1; FXR2; mGLUR5 |
| Syndrome | |
| Secretase | APH-1 (alpha and beta); Presenilin (Psen1); nicastrin |
| Related | (NcsTn); PEN-2 |
| Disorders | |
| Prion - related | Prp |
| disorders | |
| ALS | SOD1; ALS2; STEX; FUS; TARDBP; VEGF (VEGF-a; |
| | VEGF-b; VEGF-C) |
| Drug | Prkce (alcohol); Drd2; Drd4; ABAT (alcohol); GRIA2; |
| addiction | Grm5; Grin1; Htr1b; Grin2a; Drd3; Pdyn; Grial (alcohol) |
| Autism | Mecp2; BZRAP1; MDGA2; Sema5A; Neurexin 1; Fragile |
| | X (FMR2 (AFF2); FXR1; FXR2; Mg1ur5) |
| Alzheimer's | E1; CHIP; UCH; UBB; Tau; LRP; PICALM; Clusterin; |
| | PS1; |
| Disease | SORL1; CR1; Vldlr; Uba1; Uba3; CHIP28 (Aqp1, |
| | Aquaporin 1); Uchl1; Uchl3; APP |
| Inflammation | IL-I0; IL-1 (IL-la; IL-1b); IL-13; IL-17 (IL-17a (CTLA8); |
| | IL-17b; IL-17c; IL-17d; 1L-17f); II-23; Cx3cr1; ptpn22; |
| | TNFa; NOD2/CARD15 for IBD: IL-6; 1L-12 (IL-12a; |
| | 1L-12b); CTLA4; Cx3cl1 |
| Parkinson's | x-Synuclein; DJ-1; LRRK2; Parkin; PINK1 |
| Disease | |

In some embodiments, a method further comprises the step of diagnosing a subject based on identifying a sequence variant, such as diagnosing the subject with a disease associated with a detected causal genetic variant, or reporting a likelihood that the patient has or will develop such disease. Examples of diseases, associated genes, and associated sequence variants are provided herein. In some embodiments, a result is reported via a report generator, such as described herein.

In some embodiments, one or more causal genetic variants are sequence variants associated with a particular type or stage of cancer, or of cancer having a particular characteristic (e.g. metastatic potential, drug resistance, drug responsiveness). In some embodiments, the disclosure provides methods for the determination of prognosis, such as where certain mutations are known to be associated with patient outcomes. For example, ctDNA has been shown to be a better biomarker for breast cancer prognosis than the traditional cancer antigen 53 (CA-53) and enumeration of circulating tumor cells (see e.g. Dawson, et al., N Engl J Med 368:1199 (20 13)). Additionally, the methods of the present disclosure can be used in therapeutic decisions, guidance and monitoring, as well as development and clinical trials of cancer therapies. For example, treatment efficacy can be monitored by comparing patient ctDNA samples from before, during, and after treatment with particular therapies such as molecular targeted therapies (monoclonal drugs), chemotherapeutic drugs, radiation protocols, etc. or combinations of these. For example, the ctDNA can be monitored to see if certain mutations increase or decrease, new mutations appear, etc., after treatment, which can allow a physician to alter a treatment (continue, stop or change treatment, for example) in a much shorter period of time than afforded by methods of monitoring that track patient symptoms. In some embodiments, a method further comprises the step of diagnosing a subject based on an identifying step, such as diagnosing the subject with a particular stage or type of cancer associated with a detected sequence variant, or reporting a likelihood that the patient has or will develop such cancer.

For example, for therapies that are specifically targeted to patients on the basis of molecular markers (e.g. Herceptin and her2/neu status), patients are tested to find out if certain mutations are present in their tumor, and these mutations can be used to predict response or resistance to the therapy and guide the decision whether to use the therapy. Therefore, detecting and monitoring ctDNA during the course of treatment can be very useful in guiding treatment selections. Some primary (before treatment) or secondary (after treatment) cancer mutations are found to be responsible for the resistance of cancers to some therapies (Misale et al., Nature 486(7404):532 (2012)).

A variety of sequence variants that are associated with one or more kinds of cancer that may be useful in diagnosis, prognosis, or treatment decisions are known. Suitable target sequences of oncological significance that find use in the methods of the disclosure include, but are not limited to, alterations in the TP53 gene, the ALK gene, the KRAS gene, the PIK3CA gene, the BRAF gene, the EGFR gene, and the KIT gene. A target sequence the may be specifically amplified, and/or specifically analyzed for sequence variants may be all or part of a cancer-associated gene. In some embodiments, one or more sequence variants are identified in the TP53 gene. TP53 is one of the most frequently mutated genes in human cancers, for example, TP53 mutations are found in 45% of ovarian cancers, 43% of large intestinal cancers, and 42% of cancers of the upper aerodigestive track (see e.g. M. Olivier, et, al. TP53Mutations in Human Cancers: Origins, Consequences, and Clinical Use. Cold Spring Harb Perspect Biol. 2010 January; 2(1). Characterization of the mutation status ofTP53 can aid in clinical diagnosis, provide prognostic value, and influence treatment for cancer patients. For example, TP53 mutations may be used as a predictor of a poor prognosis for patients in CNS tumors derived from glial cells and a predictor of rapid disease progression in patients with chronic lymphocytic leukemia (see e.g. McLendon RE, et al. Cancer. 2005 Oct 15; 1 04(8): 1693-9; Dicker F, et al. Leukemia. 2009 Jan;23(1):117-24). Sequence variation can occur anywhere within the gene. Thus, all or part of the TP53 gene can be evaluated herein. That is, as described elsewhere herein, when target specific components (e.g. target specific primers) are used, a plurality of TP53 specific sequences can be used, for example to amplify and detect fragments spanning the gene, rather than just one or more selected subsequences (such as mutation “hot spots”) as may be used for selected targets. Alternatively, target-specific primers may be designed that hybridize upstream or downstream of one or more selected subsequences (such a nucleotide or nucleotide region associated with an increased rate of mutation among a class of subjects, also encompassed by the term “hot spot”). Standard primers spanning such a subsequence may be designed, and/or B2B primers that hybridize upstream or downstream of such a subsequence may be designed.

In some embodiments, one or more sequence variants are identified in all or part of the ALK gene. ALK fusions have been reported in as many as 7% of lung tumors, some of which are associated with EGFR tyrosine kinase inhibitor (TM) resistance (see e.g. Shaw et al., J Clin Oncol. Sep 10, 2009; 27(26): 4247-4253). Up to 2013, several different point mutations spanning across the entire ALK tyrosine kinase domain have been found in patients with secondary resistance to the ALK tyrosine kinase inhibitor (TM) (Katayama R 2012 Sci Transl Med. 2012 Feb 8;4(120)). Thus, mutation detection in ALK gene can be used to aid cancer therapy decisions.

In some embodiments, one or more sequence variants are identified in all or part of the KRAS gene. Approximately 15-25% of patients with lung adenocarcinoma and 40% of patients with colorectal cancer have been reported as harboring tumor associated KRAS mutations (see e.g. Neuman 2009, Pathol Res Pract. 2009;205(12):858-62). Most of the mutations are located at codons 12, 13, and 61 of the KRAS gene. These mutations activate KRAS signaling pathways, which trigger growth and proliferation of tumor cells. Some studies indicate that patients with tumors harboring mutations in KRAS are unlikely to benefit from anti-EGFR antibody therapy alone or in combination with chemotherapy (see e.g. Amado et al. 2008 J Clin On col. 2008 Apr 1 ;26(1 0): 1626-34, Bokemeyer et al. 2009 J Clin Oncol. 2009 Feb 10;27(5):663-71). One particular “hot spot” for sequence variation that may be targeted for identifying sequence variation is at position 35 of the gene. Identification of KRAS sequence variants can be used in treatment selection, such as in treatment selection for a subject with colorectal cancer.

In some embodiments, one or more sequence variants are identified in all or part of the PIK3CA gene. Somatic mutations in PIK3CA have been frequently found in various type of cancers, for example, in 10-30% of colorectal cancers (see e.g. Samuels et al. 2004 Science. 2004 Apr 23;304(5670):554.). These mutations are most commonly located within two “hotspot” areas within exon 9 (the helical domain) and exon 20 (the kinase domain), which may be specifically targeted for amplification and/or analysis for the detection sequence variants. Position 3140 may also be specifically targeted.

In some embodiments, one or more sequence variants are identified in all or part of the BRAF gene. Near 50% of all malignant melanomas have been reported as harboring somatic mutations in BRAF (see e.g. Maldonado et al., J Natl Cancer Inst. 2003 Dec 17;95(24):1878-90). BRAF mutations are found in all melanoma subtypes but are most frequent in melanomas derived from skin without chronic sun-induced damage. Among the most common BRAF mutations in melanoma are missense mutations V600E, which substitutes valine at position 600 with glutamine. BRAF V600E mutations are associated with clinical benefit of BRAF inhibitor therapy. Detection of BRAF mutation can be used in melanoma treatment selection and studies of the resistance to the targeted therapy.

In some embodiments, one or more sequence variants are identified in all or part of the EGFR gene. EGFR mutations are frequently associated with Non-Small Cell Lung Cancer (about 10% in the US and 35% in East Asia; see e.g. Pao et al., Proc Natl Acad Sci US A. 2004 Sep 7;101(36):13306-11). These mutations typically occur within EGFR exons 18-21, and are usually heterozygous. Approximately 90% of these mutations are exon 19 deletions or exon 21 L858R point mutations.

In some embodiments, one or more sequence variants are identified in all or part of the KIT gene. Near 85% of Gastrointestinal Stromal Tumor (GIST) have been reported as harboring KIT mutations (see e.g. Heinrich et al. 2003 J Clin Oncol. 2003 Dec I ;21 (23):4342-9). The majority of KIT mutations are found in juxtamembrane domain (exon 11, 70%), extracellular dimerization motif(exon 9, 10-15%), tyrosine kinase I (TM) domain (exon 13, 1-3%), and tyrosine kinase 2 (TK2) domain and activation loop (exon 17, 1-3%). Secondary KIT mutations are commonly identified after target therapy imatinib and after patients have developed resistance to the therapy.

Additional non-limiting examples of genes associated with cancer, all or a portion of which may be analyzed for sequence variants according to a method described herein include, but are not limited to PTEN; ATM; ATR; EGFR; ERBB2; ERBB3; ERBB4; Notch1; Notch2; Notch3; Notch4; AKT; AKT2; AKT3; HIF; HIF1a; HIF3a; Met; HRG; Bc12; PPAR alpha; PPAR gamma; WT1 (Wilms Tumor); FGF Receptor Family members (5 members: 1, 2, 3, 4, 5); CDKN2a; APC; RB (retinoblastoma); MEN1; VHL; BRCA1; BRCA2; AR; (Androgen Receptor); TSG101; IGF; IGF Receptor; Igfl (4 variants); Igf2 (3 variants); Igf 1 Receptor; Igf 2 Receptor; Bax; Bc12; caspases family (9 members: 1, 2, 3, 4, 6, 7, 8, 9, 12); Kras; and Apc. Further examples are provided elsewhere herein. Examples of cancers that may be diagnosed based on identifying one or more sequence variants in accordance with a method disclosed herein include, without limitation, Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. Non-limiting examples of specific sequence variants associated with cancer are provided in Table 3.

TABLE 3
Specific sequence variants that may be associated with cancer

| | Mutation | COSMIC | |
|---|---|---|---|
| | Amino acid | mutation % in | |
| Gene | Nucleotide | melanoma | Clinical relevance |
| BRAF | V600E | 50% (80-90% among | Increase sensitivity to BRAF |
| | 1799T>A | BRAF mutations) | inhibitors |
| | | | Increase sensitivity to MEK |
| | | | inhibitors |
| BRAF | V600E | 50% (80-90% among | Increase sensitivity to BRAF |
| | 1799_1800delTGinsAA | BRAF mutations) | inhibitors |
| | | | Increase sensitivity to MEK |
| | | | inhibitors |
| BRAF | V600R | 50% (<5% among | Increase sensitivity to BRAF |
| | 1798_1799delGTinsAG | BRAF mutations) | inhibitors |
| | | | Respond to MEK inhibitors |
| BRAF | V600M | 50% (<1% among | Increase sensitivity to BRAF |
| | 1798G>A | BRAF mutations) | inhibitors |
| | | | Respond to MEK inhibitors |
| BRAF | V600K | 50% (5% among | Increase sensitivity to BRAF |
| | 1798_1799delGTinsAA | BRAF mutations) | inhibitors |
| | | | Increase sensitivity to MEK |
| | | | inhibitors |
| BRAF | V600G | 50% (<1% among | Increase sensitivity BRAF |
| | 1799T>G | BRAF mutations) | inhibitors |
| | | | Respond to MEK inhibitors |
| BRAF | V600D | 50% (<5% among | Increase sensitivity to BRAF |
| | 1799-800delTGinsAT | BRAF mutations) | inhibitors |
| | | | Respond to MEK inhibitors |
| BRAF | L597V | 50% (1% among | Respond to BRAF inhibitors |
| | 1789C>G | BRAF mutations) | Respond to MEK inhibitors |
| BRAF | L597S | 50% (<1% among | Respond to BRAF inhibitors |
| | 1789_1790delCTinsTC | BRAF mutations) | Respond to MEK inhibitors |
| BRAF | L597Q | 50% (<1% among | Respond to BRAF inhibitors |
| | 1790T>A | BRAF mutations) | Respond to MEK inhibitors |
| BRAF | L597R | 50% (<1% among | Respond to BRAF inhibitors |
| | 1790T>G | BRAF mutations) | Respond to MEK inhibitors |
| BRAF | D594N | 50% (<1% among | Not to BRAF inhibitors |
| | 1799_1780delTGinsGA | BRAF mutations) | May respond to MEK |
| | | | inhibitors |
| BRAF | D594H | 50% (<1% among | Not to BRAF inhibitors |
| | 1780C>G | BRAF mutations) | May respond to MEK |
| | | | inhibitors |
| BRAF | D594E | 50% (<1% among | Not to BRAF inhibitors |
| | 1782T>A | BRAF mutations) | May respond to MEK |
| | | | inhibitors |
| BRAF | D594E | 50% (<1% among | Not to BRAF inhibitors |
| | 1782T>G | BRAF mutations) | May respond to MEK |
| | | | inhibitors |
| BRAF | D594G | 50% (<1% among | Not to BRAF inhibitors |
| | 1781A>G | BRAF mutations) | May respond to MEK |
| | | | inhibitors |
| BRAF | D594V | 50% (<1% among | Not to BRAF inhibitors |
| | 1781A>T | BRAF mutations) | May respond to MEK |
| | | | inhibitors |
| NRAS | G12D | 13-25% (4% among | cytotoxic chemotherapy |
| | 35G>A | NRAS mutations) | |
| NRAS | G13R | 13-25% (2% among | cytotoxic chemotherapy |
| | 37G>C | NRAS mutations) | Not to BRAF inhibitors |
| | | | May respond to MEK |
| | | | inhibitors |
| NRAS | G13D | 13-25% (2% among | cytotoxic chemotherapy |
| | 38G>A | NRAS mutations) | Not to BRAF inhibitors |
| | | | May respond to MEK |
| | | | inhibitors |
| NRAS | G13V | 13-25% (2% among | cytotoxic chemotherapy |
| | 38G>T | NRAS mutations) | Not to BRAF inhibitors |
| | | | May respond to MEK |
| | | | inhibitors |
| NRAS | Q61K | 13-25% (34% among | Respond to MEK inhibitors |
| | 181C>A | NRAS mutations) | |
| NRAS | Q61L | 13-25% (8% among | Respond to MEK inhibitors |
| | 182A>T | NRAS mutations) | |
| NRAS | Q61R | 13-25% (35% among | Respond to MEK inhibitors |
| | 182A>G | NRAS mutations) | |
| NRAS | Q61H | 13-25% (2% among | Respond to MEK inhibitors |
| | 183A>C | NRAS mutations) | |
| NRAS | Q61H | 13-25% (2% among | Respond to MEK inhibitors |
| | 183A>T | NRAS mutations) | |
| CTNNB1 | S37F | ~2-3% in primary | |
| | 110C>T | uveal melanoma (46% | |
| | | among CTNNB1 | |
| | | mutations) | |
| CNA11 | Q209L | 34% in primary uveal | |
| | 626A>T | melanoma (92% | |
| | | among CNA11 | |
| | | mutations) | |
| CNA11 | Q209P | 34% in primary uveal | |
| | 626A>C | melanoma (1% | |
| | | among CNA11 | |
| | | mutations) | |
| GNAQ | Q209L | ~50% in primary uveal | Sensitive to MEK inhibitors |
| | 626A>T | melanoma | |
| | | (~33% among CNAQ | |
| | | mutations) | |
| GNAQ | Q209P | ~50% in primary uveal | Sensitive to MEK inhibitors |
| | 626A>C | melanoma | Sensitive to MEK inhibitors |
| | | (~64% among CNAQ | |
| | | mutations) | |
| GNAQ | Q209R | ~50% in primary uveal | |
| | 626A>G | melanoma | |
| | | (~2% among CNAQ | |
| | | mutations) | |
| KIT | K624E | ~20% among KIT | Increase sensitivity to KIT |
| | 1924A>G | mutant malignant | inhibitor |
| | | melanomas | |
| KIT | D816H | ~5% among KIT | Increase sensitivity to KIT |
| | 2446G>C | mutant malignant | inhibitor |
| | | melanomas | |
| KIT | L567P | ~25% among KIT | Increase sensitivity to KIT |
| | 1727T>C | mutant malignant | inhibitor |
| | | melanomas | |
| KIT | V559A | ~20% among KIT | Increase sensitivity to KIT |
| | 1676T>C | mutant malignant | inhibitor |
| | | melanomas | |
| KIT | V559D | ~5% among KIT | Increase sensitivity to KIT |
| | 1676T>A | mutant malignant | inhibitor |
| | | melanomas | |
| KIT | W557R | ~10% among KIT | Increase sensitivity to KIT |
| | 1669T>C | mutant malignant | inhibitor |
| | | melanomas | |
| KIT | W557R | ~10% among KIT | Increase sensitivity to KIT |
| | 1669T>A | mutant malignant | inhibitor |
| | | melanomas | |

In addition, the methods and compositions disclosed herein may be useful in discovering new, rare mutations that are associated with one or more cancer types, stages, or cancer characteristics. For example, populations of individuals sharing a characteristic under analysis (e.g., a particular disease, type of cancer, stage of cancer, etc.) may be subjected to a method of detection sequence variants according to the disclosure so as to identify sequence variants or types of sequence variants (e.g., mutations in particular genes or parts of genes). Sequence variants identified as occurring with a statistically significantly greater frequency among the group of individuals sharing the characteristic than in individuals without the characteristic may be assigned a degree of association with that characteristic. The sequence variants or types of sequence variants so identified may then be used in diagnosing or treating individuals discovered to harbor them.

Other therapeutic applications include use in non-invasive fetal diagnostics. Fetal DNA can be found in the blood of a pregnant woman. Methods and compositions described herein can be used to identify sequence variants in circulating fetal DNA, and thus may be used to diagnose one or more genetic diseases in the fetus, such as those associated with one or more causal genetic variants. Non-limiting examples of causal genetic variants are described herein, and include trisomies, cystic fibrosis, sickle-cell anemia, and Tay-Saks disease. In this embodiment, the mother may provide a control sample and a blood sample to be used for comparison. The control sample may be any suitable tissue, and will typically be process to extract cellular DNA, which can then be sequenced to provide a reference sequence. Sequences of cfDNA corresponding to fetal genomic DNA can then be identified as sequence variants relative to the maternal reference. The father may also provide a reference sample to aid in identifying fetal sequences, and sequence variants.

Still further therapeutic applications include detection of exogenous polynucleotides, such as from pathogens (e.g. bacteria, viruses, fungi, and microbes), which information may inform a diagnosis and treatment selection. For example, some HIV subtypes correlate with drug resistance (see e.g. hivdb.stanford.edu/pages/genotype-rx). Similarly, HCV typing, subtyping and isotype mutations can also be done using the methods and compositions of the present disclosure. Moreover, where an HPV subtype is correlated with a risk of cervical cancer, such diagnosis may further inform an assessment of cancer risk. Further non-limiting examples of viruses that may be detected include Hepadnavirus hepatitis B virus (HBV), woodchuck hepatitis virus, ground squirrel (Hepadnaviridae) hepatitis virus, duck hepatitis B virus, heron hepatitis B virus, Herpesvirus herpes simplex virus (HSV) types 1 and 2, varicella-zoster virus, cytomegalovirus (CMV), human cytomegalovirus (HCMV), mouse cytomegalovirus (MCMV), guinea pig cytomegalovirus (GPCMV), Epstein-Barr virus (EBV), human herpes virus 6 (HHV variants A and B), human herpes virus 7 (HHV-7), human herpes virus 8 (HHV-8), Kaposi's sarcoma-associated herpes virus (KSHV), B virus Poxvirus vaccinia virus, variola virus, smallpox virus, monkeypox virus, cowpox virus, camelpox virus, ectromelia virus, mousepox virus, rabbitpox viruses, raccoonpox viruses, molluscum contagiosum virus, orf virus, milker's nodes virus, bovin papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, Flavivirus dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Togavirus, Venezuelan equine encephalitis (VEE) virus, chikungunya virus, Ross River virus, Mayaro virus, Sindbis virus, rubella virus, Retrovirus human immunodeficiency virus (HIV) types 1 and 2, human T cell leukemia virus (HTLV) types 1, 2, and 5, mouse mammary tumor virus (MMTV), Rous sarcoma virus (RSV), lentiviruses, Coronavirus, severe acute respiratory syndrome (SARS) virus, Filovirus Ebola virus, Marburg virus, Metapneumoviruses (MPV) such as human metapneumovirus (HMPV), Rhabdovirus rabies virus, vesicular stomatitis virus, Bunyavirus, Crimean-Congo hemorrhagic fever virus, Rift Valley fever virus, La Crosse virus, Hantaan virus, Orthomyxovirus, influenza virus (types A, B, and C), Paramyxovirus, parainfluenza virus (PIV types 1, 2 and 3), respiratory syncytial virus (types A and B), measles virus, mumps virus, Arenavirus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Punta toro virus (PTV), Tacaribe virus and Tamiami virus.

Examples of bacterial pathogens that may be detected by methods of the disclosure include, without limitation, Specific examples of bacterial pathogens include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Moraxella catarrhalis, Morganella sp., Mobiluncus sp., Micrococcus sp., Mycobacterium sp. (such as Mycobacterium leprae, Mycobacterium tuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaminogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsia, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

In some embodiments, the methods and compositions of the disclosure are used in monitoring organ transplant recipients. Typically, polynucleotides from donor cells will be found in circulation in a background of polynucleotides from recipient cells. The level of donor circulating DNA will generally be stable if the organ is well accepted, and the rapid increase of donor DNA (e.g. as a frequency in a given sample) can be used as an early sign of transplant rejection. Treatment can be given at this stage to prevent transplant failure. Rejection of the donor organ has been shown to result in increased donor DNA in blood; see Snyder et al., PNAS 108(15):6629 (2011). The present disclosure provides significant sensitivity improvements over prior techniques in this area. In this embodiment, a recipient control sample (e.g. cheek swab, etc.) and a donor control sample can be used for comparison. The recipient sample can be used to provide that reference sequence, while sequences corresponding to the donor's genome can be identified as sequence variants relative to that reference. Monitoring may comprise obtaining samples (e.g. blood samples) from the recipient over a period of time. Early samples (e.g. within the first few weeks) can be used to establish a baseline for the fraction of donor cfDNA. Subsequent samples can be compared to the baseline. In some embodiments, an increase in the fraction of donor cfDNA of about or at least about 10%, 20%, 30%, 40%, 50%, 60%, 70%, 80%, 90%, 95%, 100%, 250%, 500%, 1000%, or more may serve as an indication that a recipient is in the process of rejecting donor tissue.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: ddPCR Analysis of Cancer Variants Using WGA Amplified Short Fragmented cfDNA Reference Standard Samples

cfDNA reference standards were made by mixing short, fragmented DNA of size ˜150 bp from different cancer cell lines and NA12878 at different ratios. Four different cfDNA reference standards were used in this study: 5 ng of 0.25% reference standard; 10 ng of 0.25% reference standard; 20 ng of 0.1% reference standard; and 20 ng of 0% reference standard.

Each sample had 3 replicates. DNA samples were denatured at 96° C. for 30 seconds, and chilled on an ice block for 2 minutes. The addition of ligation mix (2 μL of 10× CircLigase buffer, 4 μL SM Betaine, 1 μL of 50 mM MnCh, 1 μL of CircLigase II (Epicentre # CL9025K) was set up on a cool block, and ligation was performed at 60° C. for 3 hours. Ligation DNA mixture was incubated at 80° C. for 45 seconds on a PCR machine, followed by an Exonuclease treatment. 1 μL Exo nuclease mix (Exol 20U/μL: ExoIII1OOU/μL=1:2) was added to each tube, and reactions were incubated at 37° C. for 30 minutes. Ligation mix was denatured at 95° C. for 2 minutes and cooled to 4° C. on ice before added to the Ready-To-Go GenomiPhi V3 cake (WGA). The WGA reaction was incubated at 30° C. for 4.5 hours, followed by heat inactivation at 65° C. for 10 minutes.

WGA products were bead purified using AmpureXP magnetic beads and sonicated to an average size of 800 bp. Aliquots of the sonicated DNA samples were then used as input for ddPCR analysis for the following variants: EGFRL858R; EGFR719S; and EGFRT790M. The Taqman primer and probe sequences used for this assay are provided in Table 4. A droplet digital PCR reaction was run according to manufacturer's specifications. (QX200™ Droplet Digital™ PCR system, Bio-Rad Laboratories)

TABLE 4
Taqman primer and probe sequences used to detect EGFR sequence variants
according to the methods provided herein.

| | | | Wild-type | Mutant |
|---|---|---|---|---|
| | | | Reporter | Reporter |
| | Forward Primer Sequence | Reverse Primer Sequence | Sequence (5′ | Sequence (5′ to |
| Variant | (5′ to 3′) | (5′ to 3′) | to 3′) | 3′) |
| EGFR_L858R | GCAGCATGTCAAGATC | CCTCCTTCTGCATGGT | AGTTTGGCC | AGTTTGGCCC |
| | ACAGATT | ATTCTTTCT | AGCCCAA | GCCCAA |
| EGFR_T790M | GCCTGCTGGGCATCTG | TCTTTGTGTTCCCGGA | ATGAGCTG | ATGAGCTGCA |
| | | CATAGTC | CGTGATGA | TGATGAG |
| | | | G | |
| EGFR_G719S | GCTCTCTTGAGGATCTT | GCCAGGGACCTTACCT | AAGTGCTG | AAGTGCTGAG |
| | GAAGGAAA | TATACAC | GGCTCCG | CTCCG |

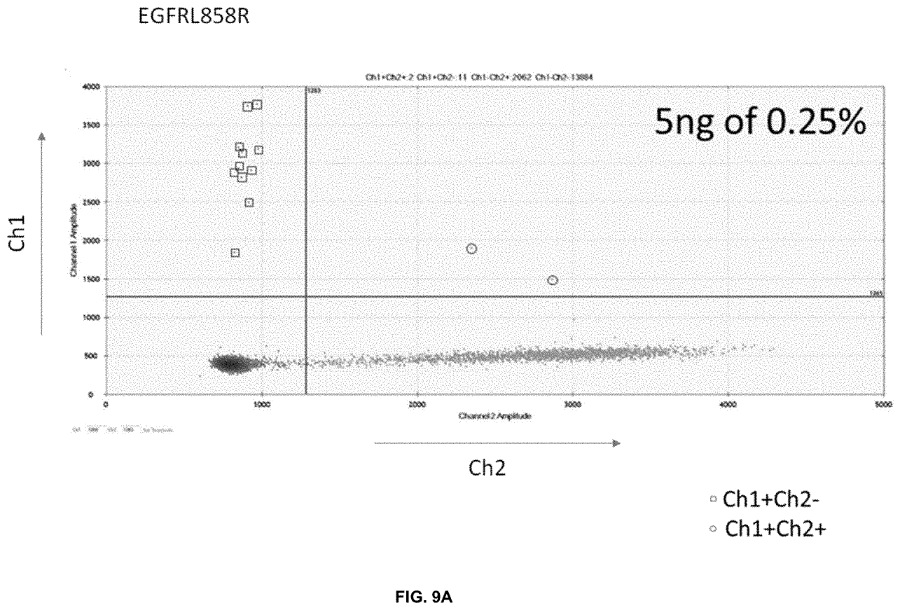

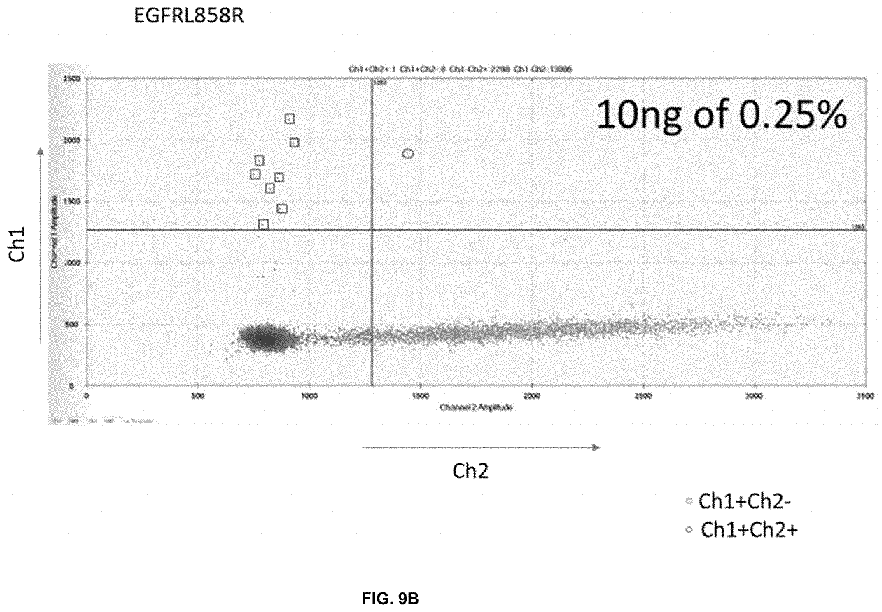

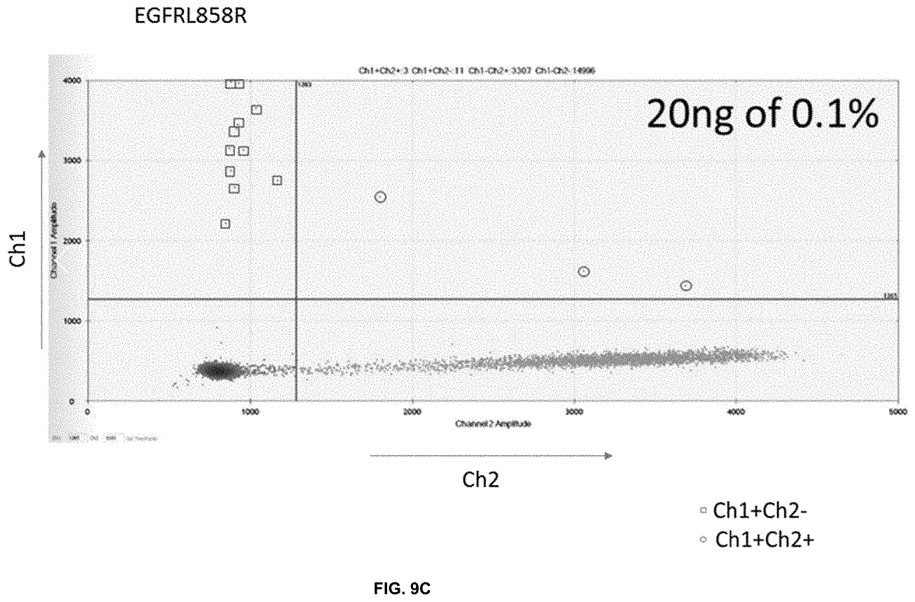

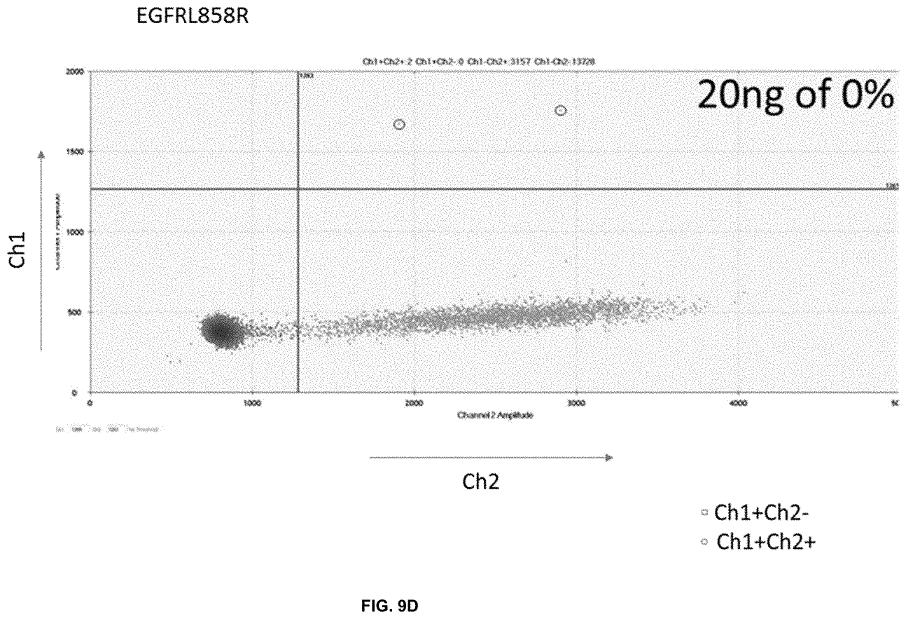

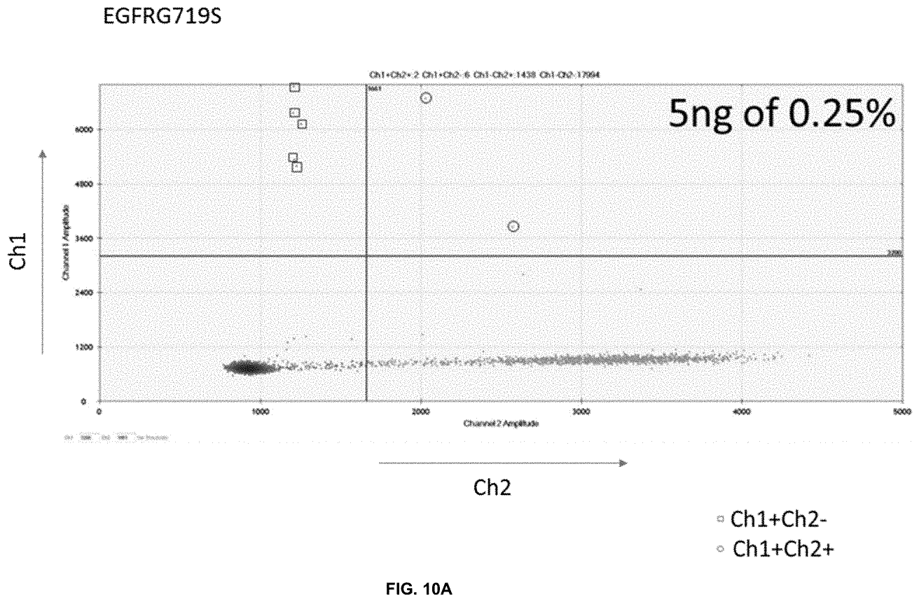

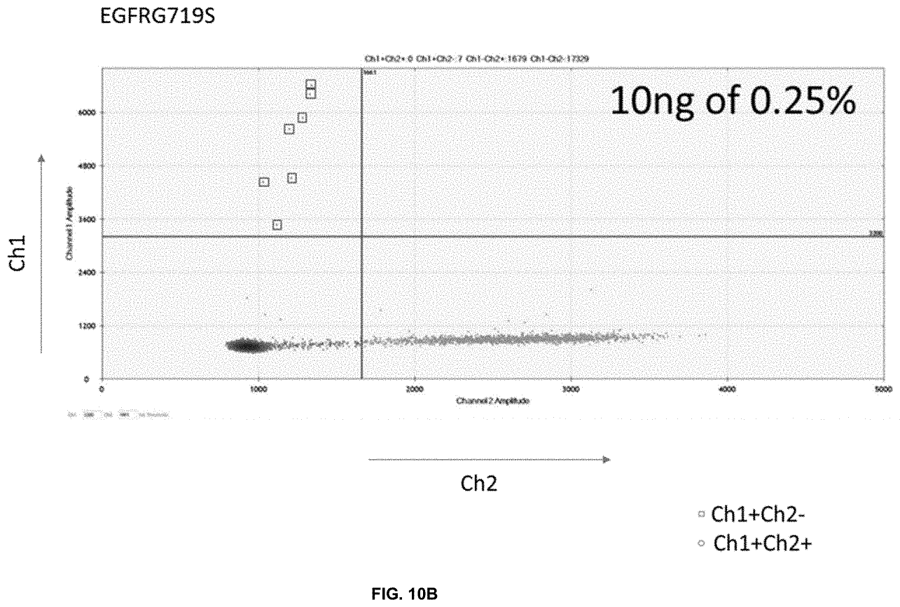

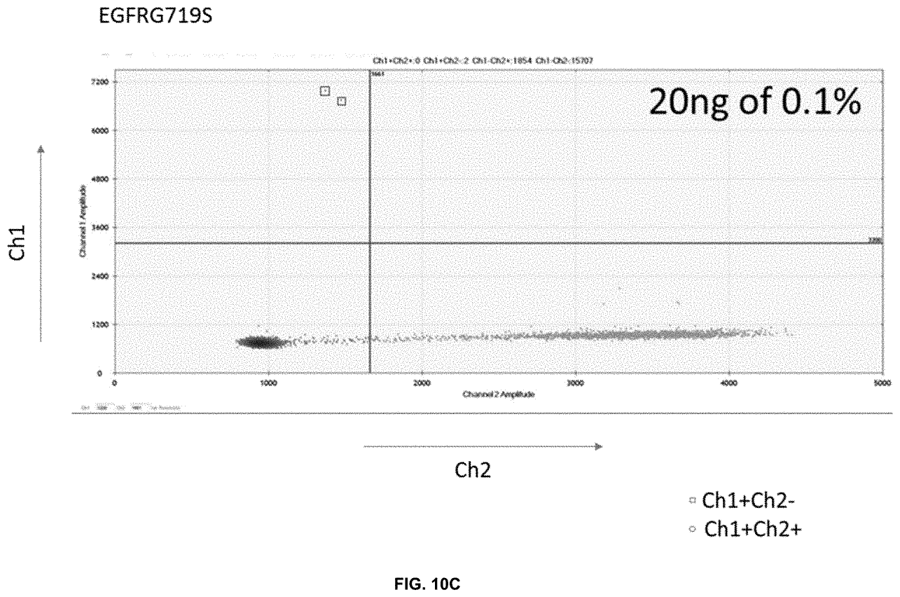

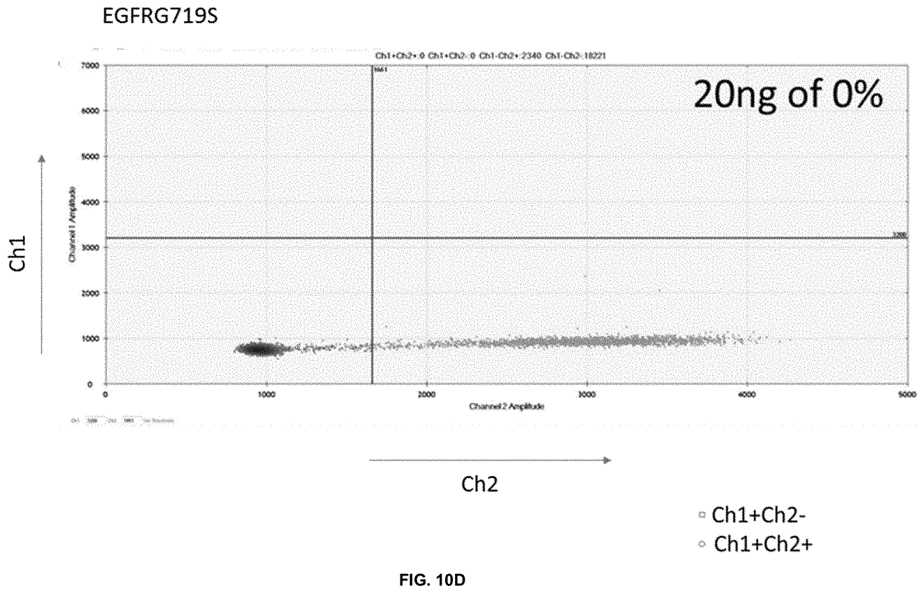

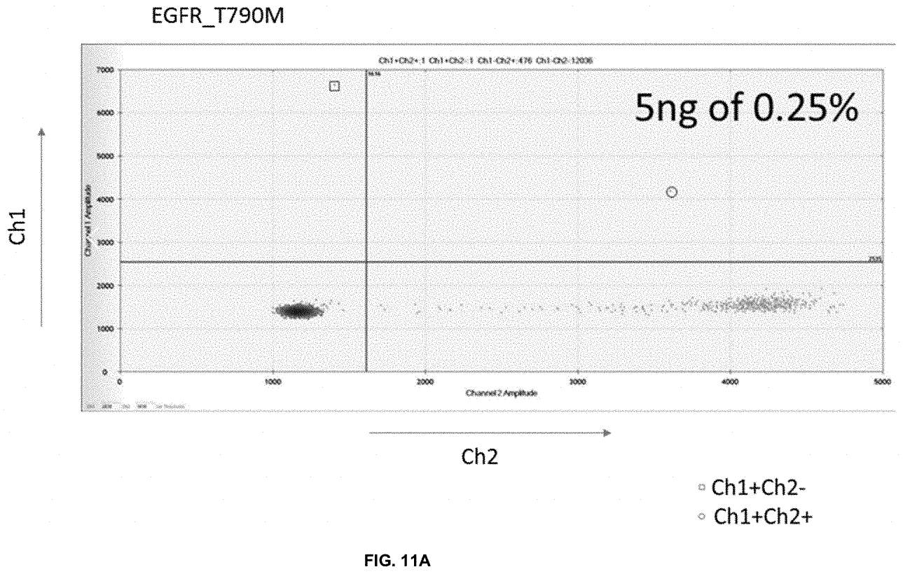

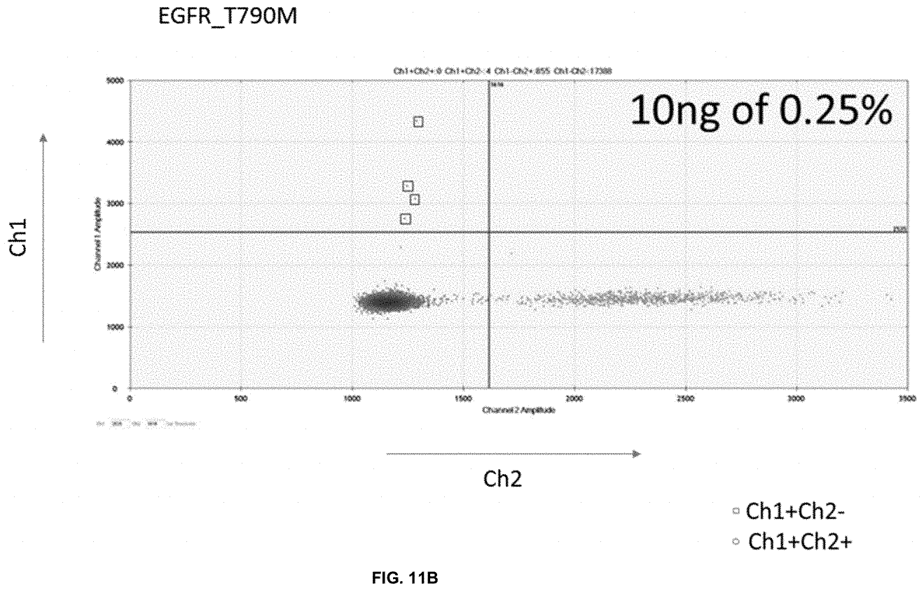

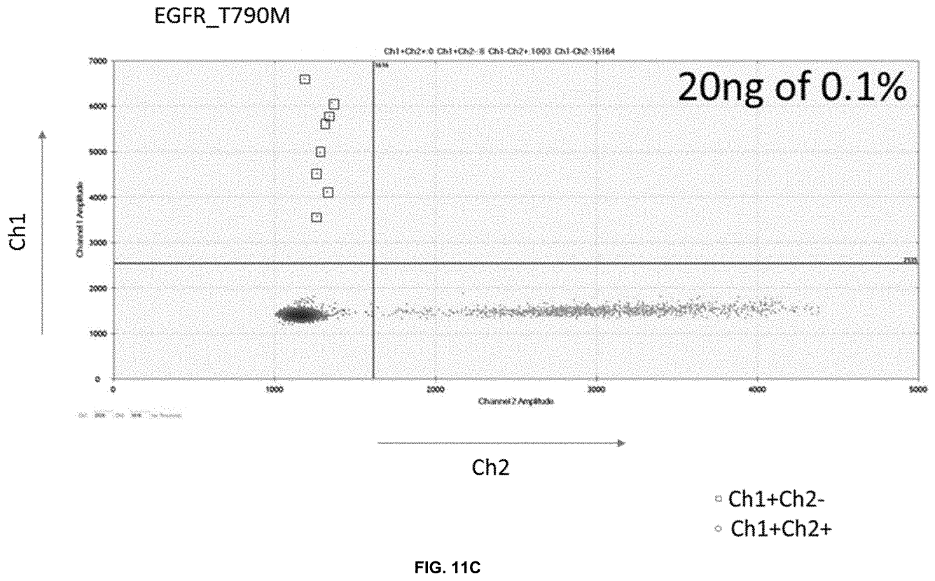

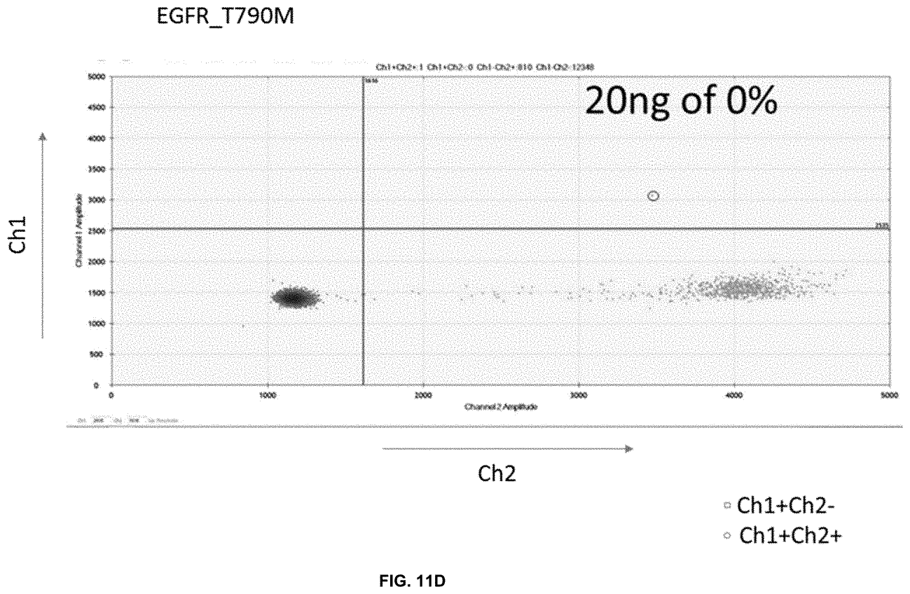

FIGS. 9A-9D, FIGS. 10A-10D, and FIGS. 11A-11D depict results obtained from the digital PCR assays. FIG. 9A, FIG. 9B, FIG. 9C, and FIG. 9D depict results obtained for digital PCR assays to identify the sequence variant EGFRL858R. FIG. 10A, FIG. 10B, FIG. 10C, and FIG. 10D depict results obtained for digital PCR assays to identify the sequence variant EGFRG719S. FIG. 11A, FIG. 11B, FIG. 11C, and FIG. 11D depict results obtained for digital PCR assays to identify the sequence variant EGFR_T790M. Individual dots in the graphs correspond to individual droplet partitions, each containing, on average, one concatemer comprising a target sequence. The Y-axis corresponds to the level of signal measured in Channel 1 (FAM), which is proportional to the amount of mutant amplicon generated in an individual partition. The X-axis corresponds to the level of signal measured in Channel 2 (HEX), which is proportional to the amount of wild-type amplicon generated in an individual partition. Threshold levels for each channel were set by the user, according to manufacturer's specifications (QX200™ Droplet Digital™ PCR system, Bio-Rad Laboratories).

Droplets that produced a signal in Channel 2 (wild-type probe) that exceeded a threshold level (e.g., were positive), and that failed to produce a signal in Channel 1 (mutant probe) that exceeded a threshold level (e.g., were negative) were considered to contain wild-type copies. Droplets that produced a signal in Channel 1 (mutant probe) that exceeded a threshold level (e.g., were positive), and that failed to produce a signal in Channel 2 (mutant probe) that exceeded a threshold level (e.g., were negative) were considered to contain mutant copies (depicted in FIGS. 9A-9D, FIGS. 10A-10D, and FIGS. 11A-11D as squares drawn around individual dots). Droplets that produced a signal in Channel 2 (wild-type probe) that exceeded a threshold level (i.e., were positive), and that produced a signal in Channel 1 (mutant probe) that exceeded a threshold level (e.g., were positive) were considered to contain a false positive and were excluded from the analysis (depicted in FIGS. 9A-9D, FIGS. 10A-10D, and FIGS. 11A-11D as circles drawn around individual dots). The average detection rate was calculated for each input amount and allele frequency, as depicted in Table 5. No false positive calls were detected in any of the blank samples.

TABLE 5

| cfDNA input (ng) | Input AF | Avg Det Rate |
|---|---|---|
| 20 | 0.10% | 100.00% |
| 10 | 0.25% | 100.00% |
| 5 | 0.25% | 100.00% |
| 20 | 0% | 0% |

Additional sequence variants may be detected using the methods described in Example 1. Non-limiting examples of mutant and wild-type probes, along with forward and reverse primers that may be used to detect additional sequence variants are provided in Table 6.

TABLE 6
Taqman primer and probe sequences that may be used to detect sequence
variants according to the methods provided herein.

| | | | Wild-type | Mutant |
|---|---|---|---|---|
| | | | Reporter | Reporter |
| | Forward Primer Sequence | Reverse Primer | Sequence (5′ to | Sequence (5′ to |
| Variant | (5′ to 3′) | Sequence (5′ to 3′) | 3′) | 3′) |
| BRAF_V600E | CATGAAGACCTCACAGT | TGGGACCCACTCC | CTAGCTACAGT | TAGCTACAGA |
| | AAAAATAGGTGAT | ATCGA | GAAATC | GAAATC |
| KIT_D816V | CTCATGGTCGGATCACA | GGGTACTCACGTT | TAGCCAGAGA | TAGCCAGAGT |
| | AAGATTTG | TCCTTTAACCA | CATCAAG | CATCAAG |
| KRAS_G13D | AGGCCTGCTGAAAATGA | CTGAATTAGCTGT | TCTTGCCTACG | CTTGCCTACG |
| | CTGAATAT | ATCGTCAAGGCA | TCACCAG | CCACCAG |
| KRAS_G12D | AGGCCTGCTGAAAATGA | GCTGTATCGTCAA | TTGGAGCTGGT | TTGGAGCTGA |
| | CTGAATAT | GGCACTCTT | GGCGTA | TGGCGTA |
| N-Ras_Q61K | GGTGAAACCTGTTTGTT | CCTGTCCTCATGT | CTGTACTCTTC | CTGTACTCTT |
| | GGACATAC | ATTGGTCTCTCA | TTGTCCAGC | CTTTTCCAGC |
| PI3Ka_H1047 | GCAAGAGGCTTTGGAGT | GCTGTTTAATTGT | CCACCATGAT | CACCATGACG |
| R | ATTTCATG | GTGGAAGATCCAA | GTGCATC | TGCATC |
| PI3Ka_E545K | TCAAAGCAATTTCTACA | GCACTTACCTGTG | CTCTCTGAAAT | CTCTGAAATC |
| | CGAGATCCT | ACTCCATAGAAA | CACTGAGCAG | ACTAAGCAG |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of identifying a sequence variant in a nucleic acid sample comprising a plurality of polynucleotides, said method comprising:
(a) circularizing said plurality of polynucleotides to form a plurality of circularized polynucleotides;
(b) amplifying said plurality of circularized polynucleotides to generate a plurality of concatemers, each comprising a plurality of sequence repeats;
(c) partitioning said plurality of concatemers into a plurality of partitions, such that, on average, no more than one concatemer comprising a target sequence is present in an individual partition,
wherein said individual partition of said plurality of partitions contains at least one of a first probe and a second probe, wherein said first probe binds to said target sequence that lacks said sequence variant and produces a first signal, and said second probe binds to said target sequence that contains said sequence variant and produces a second signal;
(d) detecting said first signal and said second signal from said individual partition; and
(e) identifying said sequence variant as present in said target sequence only when a level of said second signal exceeds that of a threshold level indicative of one copy of a target sequence, and a level of said first signal is below that of a threshold level indicative of one copy of a target sequence.

2. The method of claim 1, further comprising, identifying said sequence variant as absent when a level of said first signal exceeds that of a threshold level indicative of one copy of a target sequence and a level of said second signal is below that of a threshold level indicative of one copy of a target sequence.

3. The method of claim 1, further comprising, identifying a false positive when a level of said first signal exceeds that of a threshold level indicative of one copy of a target sequence, and a level of said second signal exceeds that of a threshold level indicative of one copy of a target sequence.

4.-5. (canceled)

6. The method of claim 1, wherein said plurality of polynucleotides comprise single-stranded polynucleotides.

7. The method of claim 1, wherein said plurality of polynucleotides comprise cell-free DNA.

8. The method of claim 1, wherein said circularizing comprises ligating a 5′ end and a 3′ end of at least one of said plurality of polynucleotides.

9. The method of any onc of claim 1, wherein said circularizing comprises ligating an adapter to the 5′ end, the 3′ end, or both the 5′ end and the 3′ end of at least one of said plurality of polynucleotides.

10. The method of any onc of claim 1, wherein said amplifying comprises amplifying using a polymerase having strand-displacement activity.

11. (canceled)

12. The method of claim 1, wherein said amplifying comprises subjecting said plurality of circular polynucleotides to an amplification reaction mixture comprising random primers.

13. The method of claim 1, wherein said amplifying comprises subjecting said plurality of circular polynucleotides to an amplification reaction mixture comprising one or more primers, each of which specifically hybridizes to a different target sequence via sequence complementarity.

14. (canceled)

15. The method of claim 1, further comprising, prior to said partitioning, fragmenting said plurality of concatemers to generate a plurality of fragmented concatemers.

16. The method of claim 15, further comprising, after said fragmenting and prior to said partitioning, selecting a plurality of said fragmented concatemers based on size.

17. The method of claim 1, wherein said plurality of partitions comprise emulsion-based droplets.

18. (canceled)

19. The method of claim 1, wherein said plurality of partitions comprise a well or a tube.

20. The method of claim 1, wherein said first probe comprises a first detectable label and said second probe comprises a second detectable label.

21.-24. (canceled)

25. The method of claim 1, wherein said first probe and said second probe are Taqman assay-based probes.

26. The method of claim 25, further comprising, after said partitioning and before said detecting, performing a polymerase chain reaction on said concatemers to amplify a region of said plurality of sequence repeats.

27. A method for reducing error in a digital polymerase chain reaction on a nucleic acid sample comprising less than 50 ng of polynucleotides, said method comprising:
(a) circularizing individual polynucleotides in said nucleic acid sample to generate a plurality of circularized polynucleotides;
(b) amplifying said plurality of circularized polynucleotides to form a plurality of concatemers, each comprising a plurality of sequence repeats;
(c) partitioning said plurality of concatemers into a plurality of partitions, such that, on average, no more than one concatemer comprising a target sequence is present in an individual partition,
wherein an individual partition of said plurality of partitions contains at least one of a first probe and a second probe, wherein said first probe binds to said plurality of sequence repeats that lack said sequence variant and produces a first signal, and said second probe binds to said plurality of sequence repeats that contain said sequence variant and produces a second signal;
(d) detecting said first signal and said second signal from said individual partition; and
(e) identifying a false positive when a level of said first signal exceeds that of a threshold level indicative of one copy of a target sequence, and a level of said second signal exceeds that of a threshold level indicative of one copy of a target sequence.

28.-29. (canceled)

30. The method of claim 27, wherein said method reduces false positives by at least 20%.

31. The method of claim 27, wherein said nucleic acid sample comprises cell-free polynucleotides.

32.-41. (canceled)


SEQUENCE LISTING (41 sequences)

SEQ ID NO: 1 - DNA, 10 bp, Unknown
  Description of Unknown HBB sequence
cagctaccat

SEQ ID NO: 2 - DNA, 10 bp, Unknown
  Description of Unknown HBB sequence
caggtaccat

SEQ ID NO: 3 - DNA, 23 bp, Artificial Sequence
  Description of Artificial Sequence Synthetic primer
gcagcatgtcaagatcacagatt

SEQ ID NO: 4 - DNA, 16 bp, Artificial Sequence
  Description of Artificial Sequence Synthetic primer
gcctgctgggcatctg

SEQ ID NO: 5 - DNA, 25 bp, Artificial Sequence
  Description of Artificial Sequence Synthetic primer
gctctcttgaggatcttgaaggaaa

SEQ ID NO: 6 - DNA, 25 bp, Artificial Sequence
  Description of Artificial Sequence Synthetic primer
cctccttctgcatggtattctttct

SEQ ID NO: 7 - DNA, 23 bp, Artificial Sequence
  Description of Artificial Sequence Synthetic primer
tctttgtgttcccggacatagtc

SEQ ID NO: 8 - DNA, 23 bp, Artificial Sequence
  Description of Artificial Sequence Synthetic primer
gccagggaccttaccttatacac

SEQ ID NO: 9 - DNA, 16 bp, Artificial Sequence
  Description of Artificial Sequence Synthetic oligonucleotide
agtttggccagcccaa

SEQ ID NO: 10 - DNA, 17 bp, Artificial Sequence
  Description of Artificial Sequence Synthetic oligonucleotide
atgagctgcgtgatgag

SEQ ID NO: 11 - DNA, 15 bp, Artificial Sequence
  Description of Artificial Sequence Synthetic oligonucleotide
aagtgctgggctccg

SEQ ID NO: 12 - DNA, 16 bp, Artificial Sequence
  Description of Artificial Sequence Synthetic oligonucleotide
agtttggcccgcccaa

SEQ ID NO: 13 - DNA, 17 bp, Artificial Sequence
  Description of Artificial Sequence Synthetic oligonucleotide
atgagctgcatgatgag

SEQ ID NO: 14 - DNA, 15 bp, Artificial Sequence
  Description of Artificial Sequence Synthetic oligonucleotide
aagtgctgagctccg

SEQ ID NO: 15 - DNA, 30 bp, Artificial Sequence
  Description of Artificial Sequence Synthetic primer
catgaagacctcacagtaaaaataggtgat

SEQ ID NO: 16 - DNA, 25 bp, Artificial Sequence
  Description of Artificial Sequence Synthetic primer
ctcatggtcggatcacaaagatttg

SEQ ID NO: 17 - DNA, 25 bp, Artificial Sequence
  Description of Artificial Sequence Synthetic primer
aggcctgctgaaaatgactgaatat

SEQ ID NO: 18 - DNA, 25 bp, Artificial Sequence
  Description of Artificial Sequence Synthetic primer
ggtgaaacctgtttgttggacatac

SEQ ID NO: 19 - DNA, 25 bp, Artificial Sequence
  Description of Artificial Sequence Synthetic primer
gcaagaggctttggagtatttcatg

SEQ ID NO: 20 - DNA, 26 bp, Artificial Sequence
  Description of Artificial Sequence Synthetic primer
tcaaagcaatttctacacgagatcct

SEQ ID NO: 21 - DNA, 18 bp, Artificial Sequence
  Description of Artificial Sequence Synthetic primer
tgggacccactccatcga

SEQ ID NO: 22 - DNA, 24 bp, Artificial Sequence
  Description of Artificial Sequence Synthetic primer
gggtactcacgtttcctttaacca

SEQ ID NO: 23 - DNA, 25 bp, Artificial Sequence
  Description of Artificial Sequence Synthetic primer
ctgaattagctgtatcgtcaaggca

SEQ ID NO: 24 - DNA, 22 bp, Artificial Sequence
  Description of Artificial Sequence Synthetic primer
gctgtatcgtcaaggcactctt

SEQ ID NO: 25 - DNA, 25 bp, Artificial Sequence
  Description of Artificial Sequence Synthetic primer
cctgtcctcatgtattggtctctca

SEQ ID NO: 26 - DNA, 26 bp, Artificial Sequence
  Description of Artificial Sequence Synthetic primer
gctgtttaattgtgtggaagatccaa

SEQ ID NO: 27 - DNA, 25 bp, Artificial Sequence
  Description of Artificial Sequence Synthetic primer
gcacttacctgtgactccatagaaa

SEQ ID NO: 28 - DNA, 17 bp, Artificial Sequence
  Description of Artificial Sequence Synthetic oligonucleotide
ctagctacagtgaaatc

SEQ ID NO: 29 - DNA, 17 bp, Artificial Sequence
  Description of Artificial Sequence Synthetic oligonucleotide
tagccagagacatcaag

SEQ ID NO: 30 - DNA, 18 bp, Artificial Sequence
  Description of Artificial Sequence Synthetic oligonucleotide
tcttgcctacgtcaccag

SEQ ID NO: 31 - DNA, 17 bp, Artificial Sequence
  Description of Artificial Sequence Synthetic oligonucleotide
ttggagctggtggcgta

SEQ ID NO: 32 - DNA, 20 bp, Artificial Sequence
  Description of Artificial Sequence Synthetic oligonucleotide
ctgtactcttcttgtccagc

SEQ ID NO: 33 - DNA, 17 bp, Artificial Sequence
  Description of Artificial Sequence Synthetic oligonucleotide
ccaccatgatgtgcatc

SEQ ID NO: 34 - DNA, 21 bp, Artificial Sequence
  Description of Artificial Sequence Synthetic oligonucleotide
ctctctgaaatcactgagcag

SEQ ID NO: 35 - DNA, 16 bp, Artificial Sequence
  Description of Artificial Sequence Synthetic oligonucleotide
tagctacagagaaatc

SEQ ID NO: 36 - DNA, 17 bp, Artificial Sequence
  Description of Artificial Sequence Synthetic oligonucleotide
tagccagagtcatcaag

SEQ ID NO: 37 - DNA, 17 bp, Artificial Sequence
  Description of Artificial Sequence Synthetic oligonucleotide
cttgcctacgccaccag

SEQ ID NO: 38 - DNA, 17 bp, Artificial Sequence
  Description of Artificial Sequence Synthetic oligonucleotide
ttggagctgatggcgta

SEQ ID NO: 39 - DNA, 20 bp, Artificial Sequence
  Description of Artificial Sequence Synthetic oligonucleotide
ctgtactcttcttttccagc

SEQ ID NO: 40 - DNA, 16 bp, Artificial Sequence
  Description of Artificial Sequence Synthetic oligonucleotide
caccatgacgtgcatc

SEQ ID NO: 41 - DNA, 19 bp, Artificial Sequence
  Description of Artificial Sequence Synthetic oligonucleotide
ctctgaaatcactaagcag